SEQUENCE LISTING (39 sequences)

SEQ ID NO: 1 - DNA, 2715 bp, Homo sapiens
gagcgaaacatctttgagcaagatgggtctctaccgcatccgcgtgtccactggggcctcgctctatgccggttccaacaaccaggtgcagctgtggctggtcggccagcacggggaggcggcgctcgggaagcgactgtggcccgcacggggcaaggagacagaactcaaggtggaagtaccggagtatctggggccgctgctgtttgtgaaactgcgcaaacggcacctccttaaggacgacgcctggttctgcaactggatctctgtgcagggccccggagccggggacgaggtcaggttcccttgttaccgctgggtggagggcaacggcgtcctgagcctgcctgaaggcaccggccgcactgtgggcgaggaccctcagggcctgttccagaaacaccgggaagaagagctggaagagagaaggaagttgtaccggtggggaaactggaaggacgggttaattctgaatatggctggggccaaactatatgacctccctgtggatgagcgatttctggaagacaagagagttgactttgaggtttcgctggccaaggggctggccgacctcgctatcaaagactctctaaatgttctgacttgctggaaggatctagatgacttcaaccggattttctggtgtggtcagagcaagctggctgagcgcgtgcgggactcctggaaggaagatgccttatttgggtaccagtttcttaatggcgccaaccccgtggtgctgaggcgctctgctcaccttcctgctcgcctagtgttccctccaggcatggaggaactgcaggcccagctggagaaggagctggagggaggcacactgttcgaagctgacttctccctgctggatgggatcaaggccaacgtcattctctgtagccagcagcacctggctgcccctctagtcatgctgaaattgcagcctgatgggaaactcttgcccatggtcatccagctccagctgccccgcacaggatccccaccacctccccttttcttgcctacggatcccccaatggcctggcttctggccaaatgctgggtgcgcagctctgacttccagctccatgagctgcagtctcatcttctgaggggacacttgatggctgaggtcattgttgtggccaccatgaggtgcctgccgtcgatacatcctatcttcaagcttataattccccacctgcgatacaccctggaaattaacgtccgggccaggactgggctggtctctgacatgggaattttcgaccagataatgagcactggtgggggaggccacgtgcagctgctcaagcaagctggagccttcctaacctacagctccttctgtccccctgatgacttggccgaccgggggctcctgggagtgaagtcttccttctatgcccaagatgcgctgcggctctgggaaatcatctatcggtatgtggaaggaatcgtgagtctccactataagacagacgtggctgtgaaagacgacccagagctgcagacctggtgtcgagagatcactgaaatcgggctgcaaggggcccaggaccgagggtttcctgtctctttacaggctcgggaccaggtttgccactttgtcaccatgtgtatcttcacctgcaccggccaacacgcctctgtgcacctgggccagctggactggtactcttgggtgcctaatgcaccctgcacgatgcggctgcccccgccaaccaccaaggatgcaacgctggagacagtgatggcgacactgcccaacttccaccaggcttctctccagatgtccatcacttggcagctgggcagacgccagcccgttatggtggctgtgggccagcatgaggaggagtatttttcgggccctgagcctaaggctgtgctgaagaagttcagggaggagctggctgccctggataaggaaattgagatccggaatgcaaagctggacatgccctacgagtacctgcggcccagcgtggtggaaaacagtgtggccatctaagcgtcgccaccctttggttatttcagcccccatcacccaagccacaagctgaccccttcgtggttatagccctgccctcccaagtcccaccctcttcccatgtcccaccctccctagaggggcaccttttcatggtctctgcacccagtgaacacattttactctagaggcatcacctgggaccttactcctctttccttccttcctcctttcctatcttccttcctctctctcttcctctttcttcattcagatctatatggcaaatagccacaattatataaatcatttcaagactagaatagggggatataatacatattactccacaccttttatgaatcaaatatgatttttttgttgttgttaagacagagtctcactttgacacccaggctggagtgcagtggtgccatcaccacggctcactgcagcctcagcgtcctgggctcaaatgatcctcccacctcagcctcctgagtagctgggactacaggctcatgccatcatgcccagctaatatttttttattttcgtggagacggggcctcactatgttgcctaggctggaaataggattttgaacccaaattgagtttaacaataataaaaagttgttttacgctaaagatggaaaagaactaggactgaactattttaaataaaatattggcaaaagaaaaaaaaaaaaaaaaaaaaaaaaa

SEQ ID NO: 2 - DNA, 2715 bp, Homo sapiens
tttttttttttttttttttttttttcttttgccaatattttatttaaaatagttcagtcctagttcttttccatctttagcgtaaaacaactttttattattgttaaactcaatttgggttcaaaatcctatttccagcctaggcaacatagtgaggccccgtctccacgaaaataaaaaaatattagctgggcatgatggcatgagcctgtagtcccagctactcaggaggctgaggtgggaggatcatttgagcccaggacgctgaggctgcagtgagccgtggtgatggcaccactgcactccagcctgggtgtcaaagtgagactctgtcttaacaacaacaaaaaaatcatatttgattcataaaaggtgtggagtaatatgtattatatccccctattctagtcttgaaatgatttatataattgtggctatttgccatatagatctgaatgaagaaagaggaagagagagaggaaggaagataggaaaggaggaaggaaggaaagaggagtaaggtcccaggtgatgcctctagagtaaaatgtgttcactgggtgcagagaccatgaaaaggtgcccctctagggagggtgggacatgggaagagggtgggacttgggagggcagggctataaccacgaaggggtcagcttgtggcttgggtgatgggggctgaaataaccaaagggtggcgacgcttagatggccacactgttttccaccacgctgggccgcaggtactcgtagggcatgtccagctttgcattccggatctcaatttccttatccagggcagccagctcctccctgaacttcttcagcacagccttaggctcagggcccgaaaaatactcctcctcatgctggcccacagccaccataacgggctggcgtctgcccagctgccaagtgatggacatctggagagaagcctggtggaagttgggcagtgtcgccatcactgtctccagcgttgcatccttggtggttggcgggggcagccgcatcgtgcagggtgcattaggcacccaagagtaccagtccagctggcccaggtgcacagaggcgtgttggccggtgcaggtgaagatacacatggtgacaaagtggcaaacctggtcccgagcctgtaaagagacaggaaaccctcggtcctgggccccttgcagcccgatttcagtgatctctcgacaccaggtctgcagctctgggtcgtctttcacagccacgtctgtcttatagtggagactcacgattccttccacataccgatagatgatttcccagagccgcagcgcatcttgggcatagaaggaagacttcactcccaggagcccccggtcggccaagtcatcagggggacagaaggagctgtaggttaggaaggctccagcttgcttgagcagctgcacgtggcctcccccaccagtgctcattatctggtcgaaaattcccatgtcagagaccagcccagtcctggcccggacgttaatttccagggtgtatcgcaggtggggaattataagcttgaagataggatgtatcgacggcaggcacctcatggtggccacaacaatgacctcagccatcaagtgtcccctcagaagatgagactgcagctcatggagctggaagtcagagctgcgcacccagcatttggccagaagccaggccattgggggatccgtaggcaagaaaaggggaggtggtggggatcctgtgcggggcagctggagctggatgaccatgggcaagagtttcccatcaggctgcaatttcagcatgactagaggggcagccaggtgctgctggctacagagaatgacgttggccttgatcccatccagcagggagaagtcagcttcgaacagtgtgcctccctccagctccttctccagctgggcctgcagttcctccatgcctggagggaacactaggcgagcaggaaggtgagcagagcgcctcagcaccacggggttggcgccattaagaaactggtacccaaataaggcatcttccttccaggagtcccgcacgcgctcagccagcttgctctgaccacaccagaaaatccggttgaagtcatctagatccttccagcaagtcagaacatttagagagtctttgatagcgaggtcggccagccccttggccagcgaaacctcaaagtcaactctcttgtcttccagaaatcgctcatccacagggaggtcatatagtttggccccagccatattcagaattaacccgtccttccagtttccccaccggtacaacttccttctctcttccagctcttcttcccggtgtttctggaacaggccctgagggtcctcgcccacagtgcggccggtgccttcaggcaggctcaggacgccgttgccctccacccagcggtaacaagggaacctgacctcgtccccggctccggggccctgcacagagatccagttgcagaaccaggcgtcgtccttaaggaggtgccgtttgcgcagtttcacaaacagcagcggccccagatactccggtacttccaccttgagttctgtctccttgccccgtgcgggccacagtcgcttcccgagcgccgcctccccgtgctggccgaccagccacagctgcacctggttgttggaaccggcatagagcgaggccccagtggacacgcggatgcggtagagacccatcttgctcaaagatgtttcgctc

SEQ ID NO: 3 - DNA, 20 bp, Artificial Sequence
  Description of Artificial Sequence Synthetic oligonucleotide
ctgaacttcttcagcacagc

SEQ ID NO: 4 - DNA, 20 bp, Artificial Sequence
  Description of Artificial Sequence Synthetic oligonucleotide
tctaaccgagctgatggact

SEQ ID NO: 5 - DNA, 20 bp, Artificial Sequence
  Description of Artificial Sequence Synthetic oligonucleotide
cagaaatcgctcatccacag

SEQ ID NO: 6 - DNA, 23 bp, Artificial Sequence
  Description of Artificial Sequence Synthetic oligonucleotide
  Description of Combined DNA/RNA Molecule Synthetic oligonucleotide
ggaguacacguuccccuguuatt

SEQ ID NO: 7 - DNA, 23 bp, Artificial Sequence
  Description of Artificial Sequence Synthetic oligonucleotide
  Description of Combined DNA/RNA Molecule Synthetic oligonucleotide
uaacaggggaacguguacucctt

SEQ ID NO: 8 - DNA, 23 bp, Artificial Sequence
  Description of Artificial Sequence Synthetic oligonucleotide
  Description of Combined DNA/RNA Molecule Synthetic oligonucleotide
guccaucagcucgguuagacutt

SEQ ID NO: 9 - DNA, 23 bp, Artificial Sequence
  Description of Artificial Sequence Synthetic oligonucleotide
  Description of Combined DNA/RNA Molecule Synthetic oligonucleotide
agucuaaccgagcugauggactt

SEQ ID NO: 10 - DNA, 23 bp, Artificial Sequence
  Description of Artificial Sequence Synthetic oligonucleotide
  Description of Combined DNA/RNA Molecule Synthetic oligonucleotide
gguggaaguaccggaguaucutt

SEQ ID NO: 11 - DNA, 23 bp, Artificial Sequence
  Description of Artificial Sequence Synthetic oligonucleotide
  Description of Combined DNA/RNA Molecule Synthetic oligonucleotide
agauacuccgguacuuccacctt

SEQ ID NO: 12 - DNA, 23 bp, Artificial Sequence
  Description of Artificial Sequence Synthetic oligonucleotide
  Description of Combined DNA/RNA Molecule Synthetic oligonucleotide
guuguacagcaugcggagagutt

SEQ ID NO: 13 - DNA, 23 bp, Artificial Sequence
  Description of Artificial Sequence Synthetic oligonucleotide
  Description of Combined DNA/RNA Molecule Synthetic oligonucleotide
acucuccgcaugcuguacaactt

SEQ ID NO: 14 - DNA, 21 bp, Artificial Sequence
  Description of Artificial Sequence Synthetic oligonucleotide
  Description of Combined DNA/RNA Molecule Synthetic oligonucleotide
gcugugcugaagaaguucatt

SEQ ID NO: 15 - DNA, 21 bp, Artificial Sequence
  Description of Artificial Sequence Synthetic oligonucleotide
  Description of Combined DNA/RNA Molecule Synthetic oligonucleotide
ugaacuucuucagcacagctt

SEQ ID NO: 16 - DNA, 21 bp, Artificial Sequence
  Description of Artificial Sequence Synthetic oligonucleotide
  Description of Combined DNA/RNA Molecule Synthetic oligonucleotide
gcugugcugaagaaguucatt

SEQ ID NO: 17 - DNA, 21 bp, Artificial Sequence
  Description of Artificial Sequence Synthetic oligonucleotide
  Description of Combined DNA/RNA Molecule Synthetic oligonucleotide
ugaacuucuucagcacagctt

SEQ ID NO: 18 - DNA, 21 bp, Artificial Sequence
  Description of Artificial Sequence Synthetic oligonucleotide
  Description of Combined DNA/RNA Molecule Synthetic oligonucleotide
guccaucagcucgguuagatt

SEQ ID NO: 19 - DNA, 21 bp, Artificial Sequence
  Description of Artificial Sequence Synthetic oligonucleotide
  Description of Combined DNA/RNA Molecule Synthetic oligonucleotide
ucuaaccgagcugauggactt

SEQ ID NO: 20 - DNA, 21 bp, Artificial Sequence
  Description of Artificial Sequence Synthetic oligonucleotide
  Description of Combined DNA/RNA Molecule Synthetic oligonucleotide
cuguggaugagcgauuucutt

SEQ ID NO: 21 - DNA, 21 bp, Artificial Sequence
  Description of Artificial Sequence Synthetic oligonucleotide
  Description of Combined DNA/RNA Molecule Synthetic oligonucleotide
agaaaucgcucauccacagtt

SEQ ID NO: 22 - DNA, 21 bp, Artificial Sequence
  Description of Artificial Sequence Synthetic oligonucleotide
  Description of Combined DNA/RNA Molecule Synthetic oligonucleotide
guccaucagcucgguuagatt

SEQ ID NO: 23 - DNA, 21 bp, Artificial Sequence
  Description of Artificial Sequence Synthetic oligonucleotide
  Description of Combined DNA/RNA Molecule Synthetic oligonucleotide
ucuaaccgagcugauggactt

SEQ ID NO: 24 - DNA, 20 bp, Artificial Sequence
  Description of Artificial Sequence Synthetic oligonucleotide
ctgaacttcttcagcacagc

SEQ ID NO: 25 - DNA, 20 bp, Artificial Sequence
  Description of Artificial Sequence Synthetic oligonucleotide
tctaaccgagctgatggact

SEQ ID NO: 26 - protein, 16 aa, Unknown
  Description of Unknown RFGF peptide sequence
AAVALLPAVLLALLAP

SEQ ID NO: 27 - protein, 11 aa, Unknown
  Description of Unknown RFGF analogue peptide sequence
AALLPVLLAAP

SEQ ID NO: 28 - protein, 13 aa, Human immunodeficiency virus
GRKKRRQRRRPPQ

SEQ ID NO: 29 - protein, 15 aa, Drosophila sp.
RQIKIWFQNRRMKWK

SEQ ID NO: 30 - DNA, 21 bp, Artificial Sequence
  Description of Artificial Sequence Synthetic oligonucleotide
  Description of Combined DNA/RNA Molecule Synthetic oligonucleotide
gcugugcugaagaaguucatt

SEQ ID NO: 31 - DNA, 21 bp, Artificial Sequence
  Description of Artificial Sequence Synthetic oligonucleotide
  Description of Combined DNA/RNA Molecule Synthetic oligonucleotide
ugaacuucuucagcacagctt

SEQ ID NO: 32 - DNA, 21 bp, Artificial Sequence
  Description of Artificial Sequence Synthetic oligonucleotide
  Description of Combined DNA/RNA Molecule Synthetic oligonucleotide
guccaucagcucgguuagatt

SEQ ID NO: 33 - DNA, 21 bp, Artificial Sequence
  Description of Artificial Sequence Synthetic oligonucleotide
  Description of Combined DNA/RNA Molecule Synthetic oligonucleotide
ucuaaccgagcugauggactt

SEQ ID NO: 34 - DNA, 21 bp, Artificial Sequence
  Description of Artificial Sequence Synthetic oligonucleotide
  Description of Combined DNA/RNA Molecule Synthetic oligonucleotide
cuguggaugagcgauuucutt

SEQ ID NO: 35 - DNA, 21 bp, Artificial Sequence
  Description of Artificial Sequence Synthetic oligonucleotide
  Description of Combined DNA/RNA Molecule Synthetic oligonucleotide
agaaaucgcucauccacagtt

SEQ ID NO: 36 - DNA, 21 bp, Artificial Sequence
  Description of Artificial Sequence Synthetic oligonucleotide
  Description of Combined DNA/RNA Molecule Synthetic oligonucleotide
ggaaaucaucuaucgguautt

SEQ ID NO: 37 - DNA, 21 bp, Artificial Sequence
  Description of Artificial Sequence Synthetic oligonucleotide
  Description of Combined DNA/RNA Molecule Synthetic oligonucleotide
auaccgauagaugauuucctt

SEQ ID NO: 38 - DNA, 21 bp, Artificial Sequence
  Description of Artificial Sequence Synthetic oligonucleotide
  Description of Combined DNA/RNA Molecule Synthetic oligonucleotide
gcacacuguucgaagcugatt

SEQ ID NO: 39 - DNA, 21 bp, Artificial Sequence
  Description of Artificial Sequence Synthetic oligonucleotide
  Description of Combined DNA/RNA Molecule Synthetic oligonucleotide
ucagcuucgaacagugugctt

(19) United States
(12) Patent Application Publication
(10) Pub. No.: US 2021/0361693 A1
(43) Pub. Date: Nov. 25, 2021

(54) PROCESS TO INHIBIT OR ELIMINATE EOSINOPHILIC DISEASES OF THE AIRWAY AND RELATED CONDITIONS

(71) Applicant: EMPIRICO INC., San Diego, CA (US)

(72) Inventors: Shannon BRUSE, San Diego, CA (US); Brian CAJES, San Diego, CA (US); Omri GOTTESMAN, San Diego, CA (US); David ROZEMA, Cross Plains, WI (US); David LEWIS, Madison, WI (US)

(21) Appl. No.: 16/967,926

(22) PCT Filed: Feb. 8, 2019
(86) PCT No.: PCT/US2019/017244

§ 371 Date: Aug. 6, 2020

Related U.S. Application Data
(60) Provisional application No. 62/628,559, filed on Feb. 9, 2018.
(60) Provisional application No. 62/677,347, filed on May 29, 2018.
(60) Provisional application No. 62/677,380, filed on May 29, 2018.
(60) Provisional application No. 62/766,594, filed on May 3, 2018.

(51) Int. Cl. A61K 31/7105; A61K 38/46; C12N 15/113; C12N 15/115; A61P 11/02

(52) U.S. Cl. CPC A61K 31/7105; A61K 38/465; A61K 48/00; C12N 15/115; A61P 11/02; C12N 15/1137

(57) ABSTRACT

Molecules for inhibiting arachidonate 15-lipoxygenase (ALOX-15) gene products including dsRNA (dsRNA) agents such as small interfering RNAs (siRNAs), antisense oligonucleotides, and small molecule inhibitors for therapeutic use. Additionally provided are methods to inhibit the expression of a target gene by administering these agents for the treatment of diseases involving ALOX-15 gene products.

### CROSS-REFERENCE

This application claims the benefit of U.S. Provisional Application No. 62/628,559 filed Feb. 9, 2018, U.S. Provisional Application No. 62/766,594 filed May 3, 2018, U.S. Provisional Application No. 62/677,347 filed May 29, 2018, and U.S. Provisional Application No. 62/677,380 filed May 29, 2018, which are each incorporated by reference herein in their entirety.

### SEQUENCE LISTING

The instant application contains a Sequence Listing which has been submitted electronically in ASCII format and is hereby incorporated by reference in its entirety. Said ASCII copy, created on Feb. 7, 2019, is named 54462-701_601_SL.txt and is 18,896 bytes in size.

### BACKGROUND

Large-scale human genetic data is now widely available and provides a mechanism for improving the success rate of pharmaceutical discovery and development by leveraging experiments of nature.

The Genome Wide Association Study (GWAS) is an experimental design to detect associations between genetic variants and traits in a population sample. The purpose is to better understand the biology of disease and to develop treatments based on this understanding. GWAS can utilize genotyping and/or sequencing data and often involves evaluation of millions of genetic variants that are relatively evenly distributed across the genome. The most common GWAS design is the case-control study, which involves comparing variant frequencies in cases versus controls. If a variant has a significantly different frequency in cases versus controls, that variant is said to be associated with disease. The commonly reported association statistics for GWAS are p-values, as a measure of statistical significance and odds ratios (OR) or beta coefficients (beta), as a measure of effect size. Researchers often assume an additive genetic model and calculate an allelic odds ratio, which is the increased (or decreased) risk of disease conferred by each additional copy of an allele (compared to carrying no copies of that allele). An additional and important concept in design and interpretation of GWAS is that of linkage disequilibrium, which is the non-random association of alleles. The presence of linkage disequilibrium can obfuscate which is the “causal” variant.

Functional annotation of variants and/or wet lab experimentation has been used to definitively identify the causal genetic variant identified via GWAS, and in many cases, this has led to the identification of disease-causing genes. In particular, understanding the functional effect of a causal genetic variant (e.g. loss or gain of protein function, increase or decrease in gene expression) allows that variant to be used as a proxy for therapeutic modulation of the target gene and to gain an insight into the potential therapeutic efficacy and safety of a therapeutic that modulates that target.

Identification of such gene-disease associations has provided fundamental insights into disease biology and is rapidly becoming an essential means of identifying novel therapeutic targets for the pharmaceutical industry. In order to translate the therapeutic insights derived from human genetics, disease biology in patients must be exogenously ‘programmed’ into replicating the observation from human genetics. Today, the potential options for therapeutic modality that could be brought to bear in translating therapeutic targets identified via human genetics into novel medicines are greater than ever before. These include well established therapeutic modalities such as small molecules and monoclonal antibodies, maturing modalities such as oligonucleotides and emerging modalities such as gene therapy and gene editing. The choice of therapeutic modality depends on several factors including the location of the target (e.g. intracellular, extracellular or secreted), the relevant tissue (e.g. lung, liver) and the relevant indication.

### SUMMARY

Chronic inflammation commonly affects both the upper and lower airways via similar mechanisms. Clinically, chronic airway inflammation often presents as allergic rhinitis (AR), non-allergic rhinitis (NAR), chronic rhinosinusitis (CRS) and nasal polyposis in the upper airway, and as asthma, Chronic Obstructive Pulmonary Disease (COPD) and the asthma-COPD overlap syndrome (ACOS) in the lower airway. These observations have fostered increasingly strong support for the so-called unified airway hypothesis. The airway is a continuous structure lined with ciliated, pseudostratified columnar epithelium that extends from the nasal vestibule to the distal bronchioles. Its mucosal surface is constantly exposed to environmental insults and is thus highly adapted in its role as the first line of defense, instigated by the innate and adaptive arms of the immune system. Though these diseases are heterogeneous in terms of their presentation and disease course, comprising many endotypes, they all share a common endotype with patients displaying a Th2-dominant response characterized by airway inflammation with local and/or systemic eosinophilia, among other features. The epidemiological and pathophysiological observations have resulted in the established dogma that the eosinophilic endotypes of airway diseases benefit from similar therapeutic approaches, revolving around modulation of the dysregulated innate, adaptive and inflammatory responses that are characteristic of these diseases.

Eosinophilic inflammation can also manifest outside of the airway, particularly in the skin as atopic dermatitis and in the gastrointestinal tract as eosinophilic esophagitis. Epidemiological and pathophysiological observations in these diseases again suggest a Th2-predominant systemic inflammatory process that shares features with eosinophilic endotypes of airway disease and may benefit from similar therapeutic approaches.

In one aspect, provided herein are therapeutic modalities to inhibit or eliminate eosinophilic diseases and related conditions, such as lipoxygenase inhibitors. Further provided are methods for making the same and methods for treating conditions mediated by lipoxygenase. Lipoxygenase enzymes play an important role in various diseases such as asthma, rheumatoid arthritis, gout, psoriasis, allergic rhinitis, Crohn's disease, respiratory distress syndrome, chronic obstructive pulmonary disease, acne, atherosclerosis, aortic aneurysm, sickle cell disease, acute lung injury, ischemia/reperfusion injury, chronic sinusitis, nasal polyposis and/or inflammatory bowel disease among others. Accordingly, in some embodiments, compositions which inhibit lipoxygenase activity are useful in the treatment and/or prevention of such diseases.

In another aspect, provided is a method of treating one or more disorders of the upper and lower airway in a subject in need thereof comprising administering to the subject an inhibitor of arachidonate 15-lipoxygenase (ALOX15) wherein the one or more disorders of the upper and lower airway comprises nasal polyposis, chronic sinusitis, allergic rhinitis, asthma, or NSAID-exacerbated respiratory disease. In some embodiments, the inhibitor of ALOX15 is delivered systemically to the subject. In some embodiments, the inhibitor of ALOX15 is delivered locally to the subject. In some embodiments, the inhibitor of ALOX15 is delivered locally to the nasal epithelium of the subject. In some embodiments, the one or more disorders of the upper and lower airway is nasal polyposis. In some embodiments, the subject has nasal polyps. In some embodiments, tissue from the subject comprising the nasal polyps comprises eosinophilic infiltration. In some embodiments, the subject has received a first line treatment comprising intranasal corticosteroids for the one or more disorders of the upper and lower airway. In some embodiments, the inhibitor of ALOX15 comprises a small molecule. In some embodiments, the inhibitor of ALOX15 comprises RNAi. In some embodiments, the RNAi inhibits translation or degrades ALOX15 mRNA. In some embodiments, the RNAi comprises siRNA, miRNA, or antisense oligonucleotide (ASO). In some embodiments, the ASO is single-stranded or double-stranded. In some embodiments, the inhibitor of ALOX15 is an aptamer. In some embodiments, the aptamer is an oligonucleotide or a peptide molecule. In some embodiments, the subject comprises an ALOX15 variant. In some embodiments, the ALOX15 variant is rs2255888. In some embodiments, the inhibitor of ALOX15 causes a reduction in the production of a metabolite of ALOX15 in the subject. In some embodiments, the metabolite of ALOX15 is 15-hydroxyeicosatetraenoic acid (15-HETE). In some embodiments, the inhibitor of ALOX15 causes a reduction in the subject of blood eosinophil counts.

In another aspect, provided is a composition comprising an inhibitor of ALOX15 that is efficacious in treating nasal polyposis, chronic sinusitis, allergic rhinitis, asthma, or NSAID-exacerbated respiratory disease. In some embodiments, the inhibitor of ALOX15 is an RNAi. In some embodiments, the RNAi is siRNA. In some embodiments, the RNAi is miRNA. In some embodiments, the RNAi is an antisense oligonucleotide (ASO). In some embodiments, the ASO is double-stranded or single-stranded. In some embodiments, the inhibitor of ALOX15 is a small molecule. In some embodiments, the inhibitor of ALOX15 is an aptamer. In some embodiments, the aptamer is an oligonucleotide aptamer. In some embodiments, the aptamer is a peptide aptamer.

In another aspect, provided is a method of treating one or more disorders of the upper and lower airway in a subject in need thereof comprising editing an ALOX15 gene in the subject wherein the one or more disorders of the upper and lower airway comprises nasal polyposis, chronic sinusitis, allergic rhinitis, asthma, or NSAID-exacerbated respiratory disease. In some embodiments, the editing of the ALOX15 gene comprises administering CRISPR/cas9 to the subject. In some embodiments, the CRISPR/cas9 targets the ALOX15 gene. In some embodiments, the CRISPR/cas9 edits the ALOX15 gene to a loss of function mutation. In some embodiments, the loss of function mutation comprises a threonine to methionine mutation. In some embodiments, the threonine to methionine mutation occurs at amino acid position 560 according to the human protein sequence numbering. In some embodiments, the CRISPR/cas9 is delivered systemically to the subject. In some embodiments, the CRISPR/cas9 is delivered locally to the subject. In some embodiments, the CRISPR/cas9 is delivered locally to the nasal epithelium of the subject. In some embodiments, the editing of the ALOX15 gene is efficacious in treating the one or more disorders of the upper and lower airway. In some embodiments, the one or more disorders of the upper and lower airway is nasal polyposis. In some embodiments, the subject has nasal polyps. In some embodiments, tissue from the subject comprising the nasal polyps comprises eosinophilic infiltration. In some embodiments, the subject has received a first line treatment comprising intranasal corticosteroids for the one or more disorders of the upper and lower airway. In some embodiments, the editing of the ALOX15 gene causes a reduction in the production of a metabolite of ALOX15 in the subject. In some embodiments, the metabolite of ALOX15 is 15-hydroxyeicosatetraenoic acid (15-HETE). In some embodiments, the editing of the ALOX15 gene causes a reduction in the subject of blood eosinophil counts.

In another aspect, provided is a composition comprising CRISPR/cas9 that targets ALOX15 that is efficacious in treating nasal polyposis, chronic sinusitis, allergic rhinitis, asthma, or NSAID-exacerbated respiratory disease. In some embodiments, the CRISPR/cas9 edits the ALOX15 gene to a loss of function mutation. In some embodiments, the loss of function mutation comprises a threonine to methionine mutation. In some embodiments, the threonine to methionine mutation occurs at amino acid position 560 according to the human protein sequence numbering.

### BRIEF DESCRIPTION OF THE DRAWINGS

FIG. 1 shows nasal polyps within the nasal cavity.

FIG. 2 is a Manhattan plot showing that about 20 genomic loci are significantly associated with diagnosis of nasal polyposis in the UK Biobank cohort.

FIG. 3 is a pathway diagram showing the metabolism of arachidonic acid and other polyunsaturated fatty acid substrates. Figure reproduced from Cornejo-Garcia, et al., 2012, Clin. & Exp. Allergy 42: 1772-81.

FIG. 4 shows data indicating ablation of ALOX15 activity for T560M. Figure reproduced from Horn, et al., 2013, Redox Biol. 1:566-77.

FIG. 5 shows data indicating an ALOX15 variant (rs2255888, 17_4545581_C_T_b37) associated with airway diseases is an expression quantitative trait locus for ALOX15. The data was obtained from the GTEx Portal.

FIG. 6 is a table showing expression of ALOX15 in nasal polyps tissue. Figure reproduced from Rostkowska, et al., 2011 Auris Nasus Larynx 38(1):58-64.

FIG. 7 shows data indicating activity of ALOX15 in nasal polyps tissue. Figure reproduced from Smith et al., Int. Arch. Allergy Appl. Immunol. 82(1):83-8.

FIG. 8 is a clinical pathology image showing increased expression of ALOX15 in eosinophilic asthma. Figure reproduced from Chu et al., 2002 November, Clin Exp Allergy 32(11):1558-65.

FIG. 9 is a clinical pathology image showing increased expression of ALOX15 in eosinophilic esophagitis. Figure reproduced from Hui Y et al., Pediatr Dev Pathol. 2016 August.

FIGS. 10A and 10B show the levels of ALOX15 mRNA (FIG. 10A) and 15(S)-HETE (FIG. 10B) in A549 cells following administration of ALOX15 siRNA.

### DETAILED DESCRIPTION

In one aspect, provided herein are molecules for inhibition of arachidonate 15-lipoxygenase (ALOX-15) gene products, including dsRNA (dsRNA) agents such as small interfering RNAs (siRNAs), antisense oligonucleotides, and small molecules for therapeutic use. Further provided are methods of inhibiting the expression of a target gene by administering a dsRNA agent, antisense oligonucleotide, or small molecule agent, e.g., for the treatment of various diseases involving ALOX-15 gene products. Also provided is a method of modulating the expression of a target gene in a cell, comprising providing to said cell a dsRNA agent, antisense oligonucleotide, or small molecule. In some embodiments, the target gene is ALOX15.

Applicant evaluated approximately 30,000,000 imputed and directly genotyped variants in ˜350,000 individuals from the UK Biobank cohort for associations with a range of chronic airway diseases in which eosinophilic endotypes are prevalent, including nasal polyps, and with blood eosinophil counts.

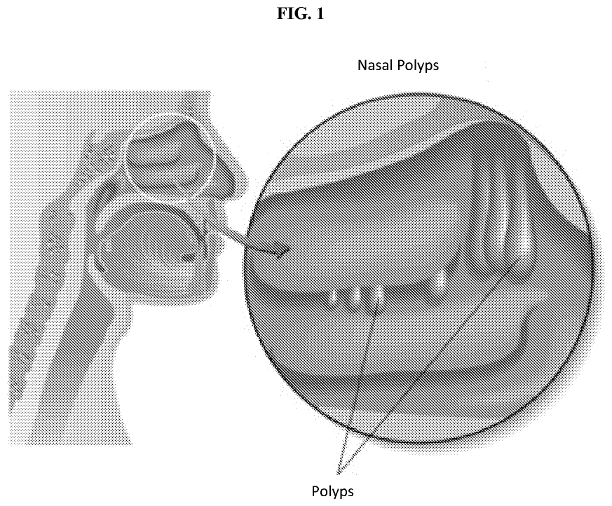

Nasal polyposis is a medical condition involving obstructive masses in the nasal cavity (see FIG. 1), leading to nasal blockage, mucus hypersecretion, and anosmia. Nasal polyps often occur in the context of chronic sinusitis, and are additionally associated with asthma, bronchiectasis, Samter's triad/NSAID-exacerbated respiratory disease, cystic fibrosis, and Kartagener's syndrome. First line treatment consists of management with intranasal corticosteroids; however, many patients require surgery, and post-surgical recurrence of polyps is common.

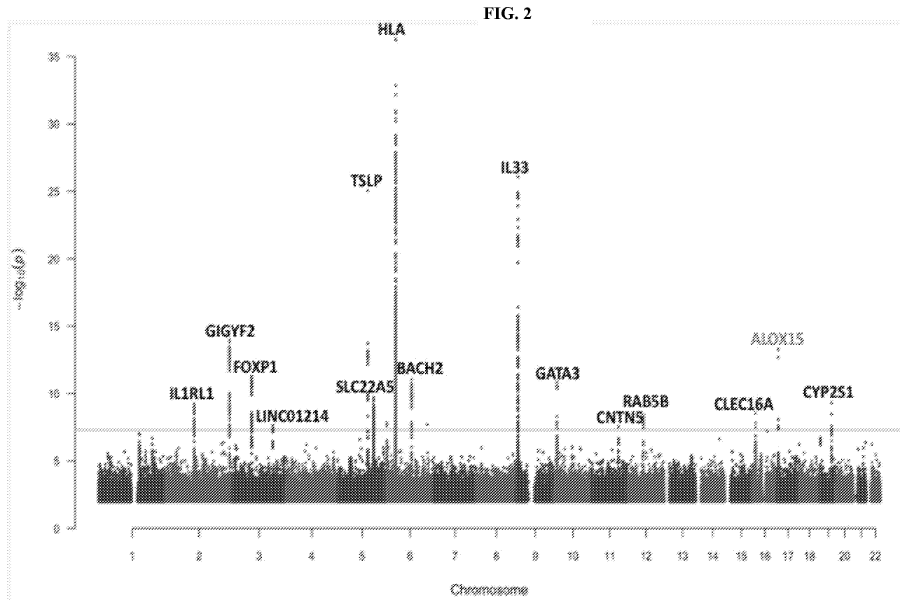

As described Example 1, about 20 genomic loci were determined to be significantly associated with diagnosis of nasal polyposis (see FIG. 2).

One cluster of association was at chromosome 17p13.2, encompassing the ALOX15 gene. The most significantly associated variant (rs34210653) at this locus was a low frequency missense variant (minor allele frequency˜1.7%) in exon 13 of the ALOX15 gene, which was consistently associated with the evaluated phenotypes (see Example 1). This variant was associated with reduced risk of nasal polyposis, and carriers of the minor allele of this variant had less than half the risk of nasal polyposis as non-carriers (p=2×10\hat{ }-15; OR=0.38). This variant was also associated with reduced risk of chronic rhinosinusitis (p=7×10\hat{ }-12; OR=0.65), allergic rhinitis (p=5×10\hat{ }-9; OR=0.80), asthma (p=9×10\hat{ }-6; OR=0.93) and reduced risk of undergoing sinus surgery including nasal polypectomy (p=5×10\hat{ }-11; OR=0.46); This variant was also associated with reduced blood eosinophil counts (p=2×10\hat{ }-65; beta=−0.02).

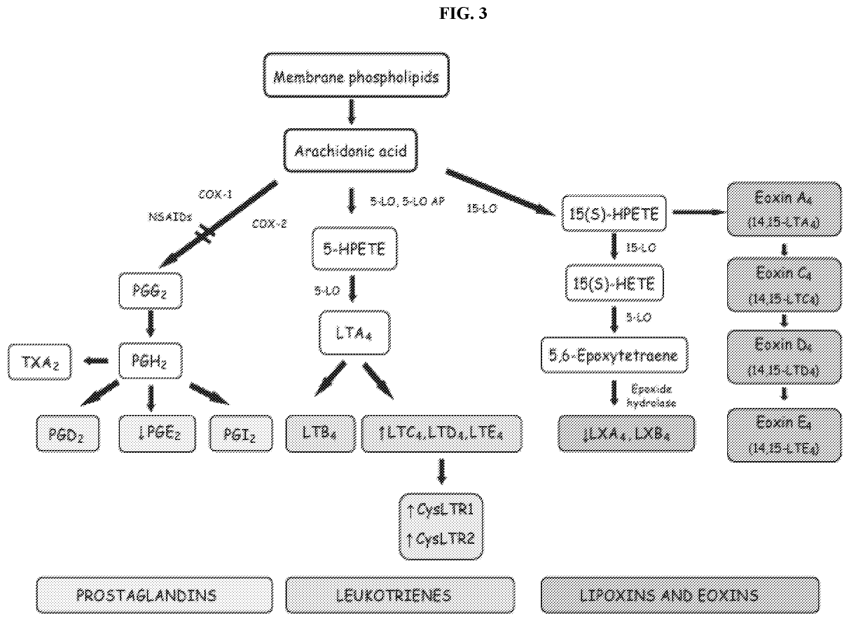

ALOX15 is one of five (ALOX5/12/12B/15/15B) human lipoxygenases and is involved in the metabolism of arachidonic acid and other polyunsaturated fatty acid substrates (FIG. 3). 15-HETE is its major arachidonic acid-derived metabolite, which is then further metabolized to eoxins, 5-oxo-15-hydroxy-ETE and other metabolites. ALOX15 metabolites are largely pro-inflammatory and have been shown to induce airway epithelial injury and promote goblet cell hyperplasia/mucus hypersecretion (15-HETE), increase vascular permeability (eoxin C4) and are potent eosinophil chemoattractants (5-oxo-15-hydroxy-ETE). ALOX15 is highly expressed in the airway and is induced in vitro by IL-13, a central mediator of the Th2 response.

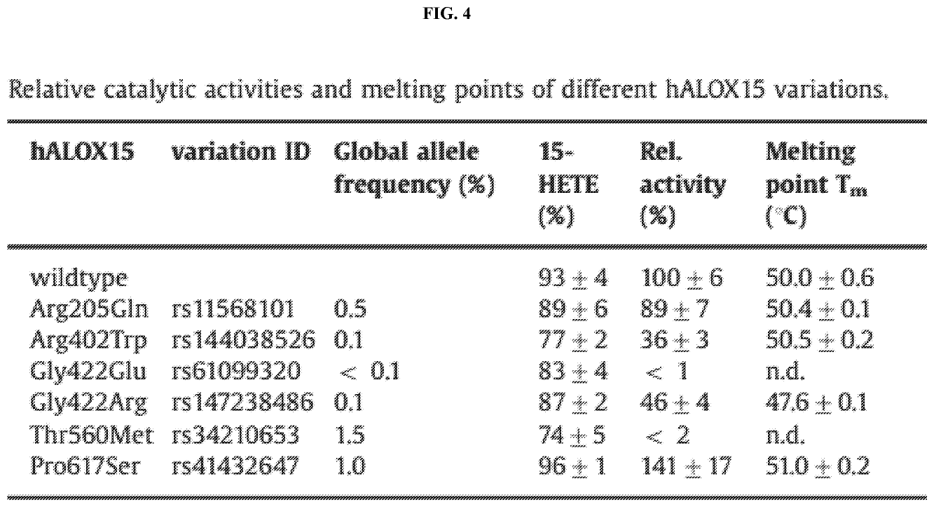

The rs34210653 variant results in a threonine to methionine change at amino acid 560 (T560M). Reports demonstrate that this T560M exchange results in near complete ablation of ALOX15 catalytic activity (FIG. 4), as measured by 15-HETE production from arachidonic acid.

By inference, these data indicate that loss of function (LOF) of ALOX15 protects against the development of nasal polyposis, chronic rhinosinusitis, allergic rhinitis and asthma. Accordingly, in some cases therapeutic inhibition of ALOX15 may be an effective genetically-informed method of treatment for these diseases.

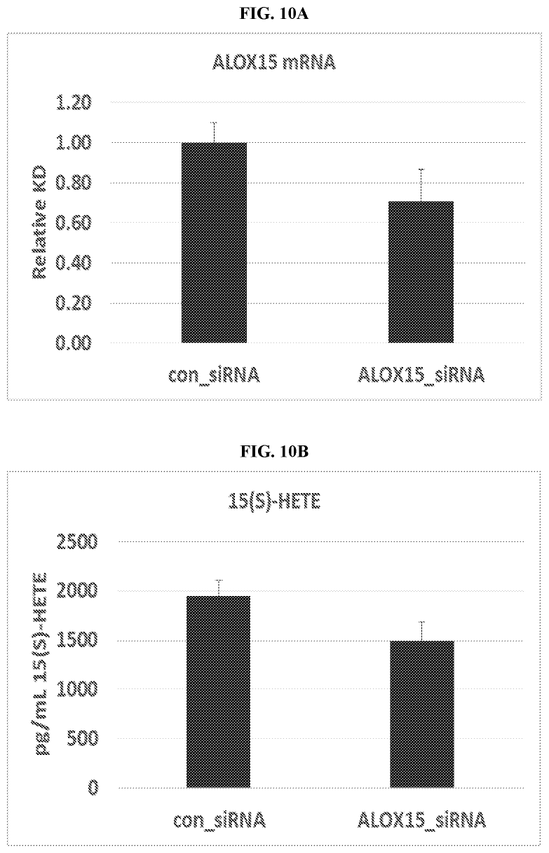

Applicant evaluated the effect of ALOX15 inhibition on 15-HETE production from arachidonic acid in cultured A549 cells (see Example 4). Two siRNAs targeted to the ALOX15 mRNA were demonstrated to downregulate levels of ALOX15 mRNA and 15-HETE (FIG. 10A, FIG. 10B), when administered to cultured A549 cells (a non-small cell lung cancer line), providing further evidence that T560M is a protective LOF variant and a valid proxy for exogenous inhibition of ALOX15.

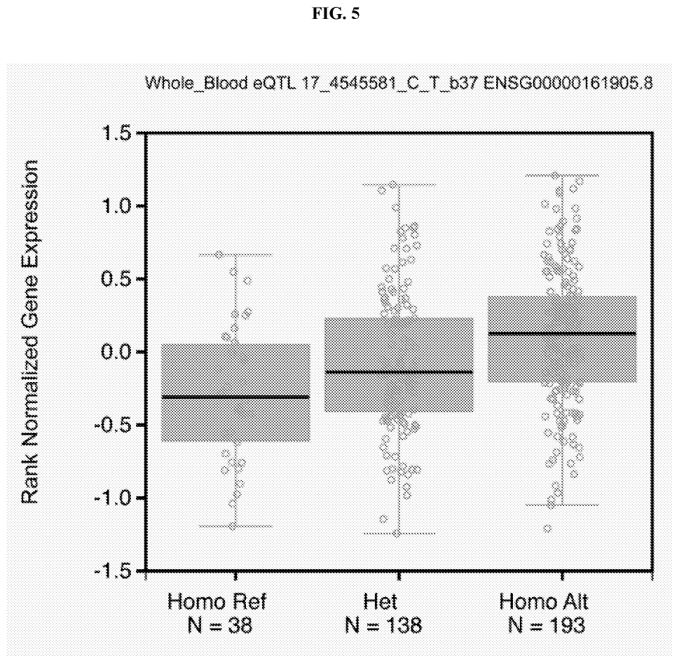

In addition to the protective LOF variant outlined above, Applicant showed that a separate, independent ALOX15 variant is associated with risk of these diseases. This variant, rs2255888, is an ALOX15 regulatory variant that is associated with increased expression of ALOX15 in whole blood (see Example 1, FIG. 5).

In an analysis conditioning on the LOF variant, the T allele of rs2255888, which is associated with increased expression of ALOX15 in whole blood, is associated with increased risk of nasal polyposis (p=7×10\hat{ }-5; OR=1.2) and increased blood eosinophil counts (p=2×10\hat{ }-22; beta=0.004). In combination with the T560M loss of function variant, Applicant has therefore identified an ALOX15 allelic series that modulates risk for nasal polyposis and blood eosinophils. This allelic series consist of an ALOX15 loss of function variant (rs34210653) that is associated with decreased risk of nasal polyposis, chronic rhinosinusitis, allergic rhinitis, asthma and decreased blood eosinophil counts, and an ALOX15 regulatory variant that increases ALOX15 expression (rs2255888) that is associated with increased risk of nasal polyposis and increased blood eosinophil counts, further suggesting therapeutic inhibition of ALOX15 as a genetically-informed method of treatment of nasal polyposis and related eosinophilic diseases of the airway.

Applicant also identified three additional rare variants that may be protein truncating or damaging to the protein and analyzed them collectively in a gene burden test (see Example 1). In aggregate these variants are associated with decreased risk of nasal polyposis (p=0.0008; OR=0.53) and decreased blood eosinophil counts (p=1.4×10\hat{ }-9; beta=−0.016).

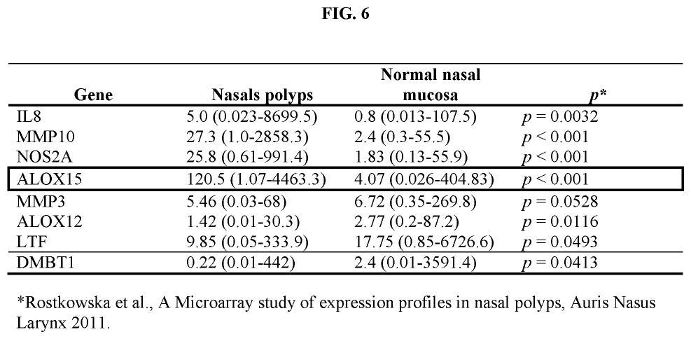

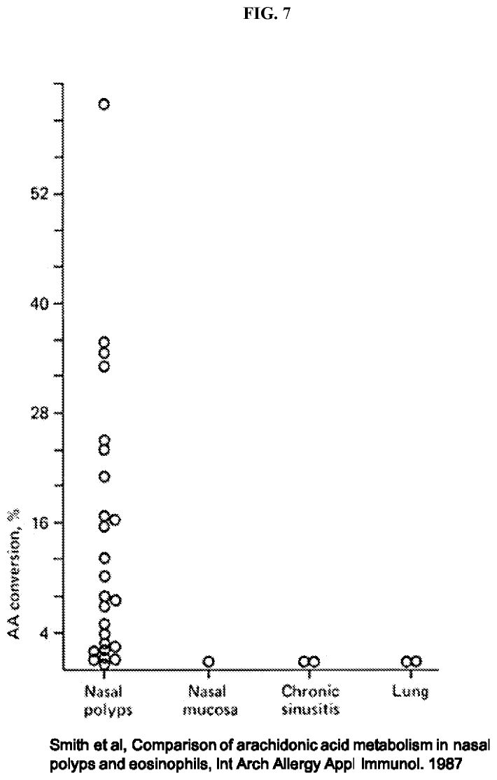

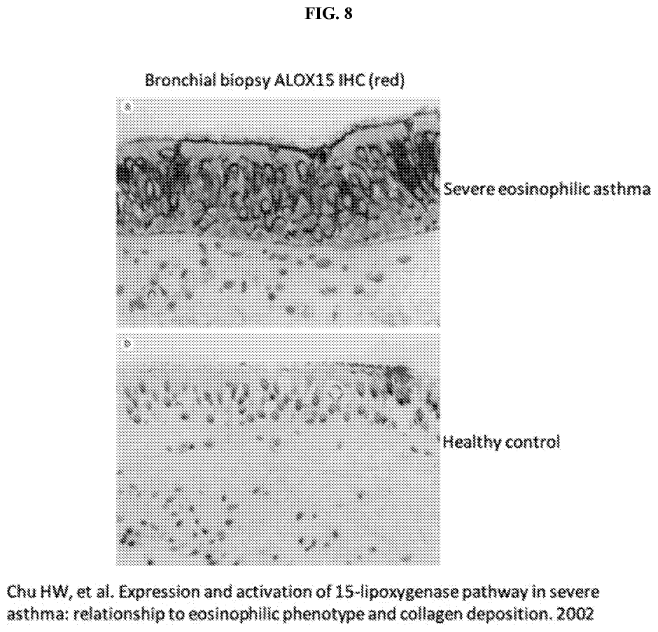

ALOX15 is highly expressed in airway epithelial cells, eosinophils and particularly in nasal polyp tissue. Both expression (FIG. 6) and activity (FIG. 7) of ALOX15 are ˜30 times greater in nasal polyps tissue than in normal mucosa. ALOX15 expression is also increased in lung granulocytes and bronchial biopsies of asthmatic patients (FIG. 8), and in bronchial biopsies of patients with COPD.

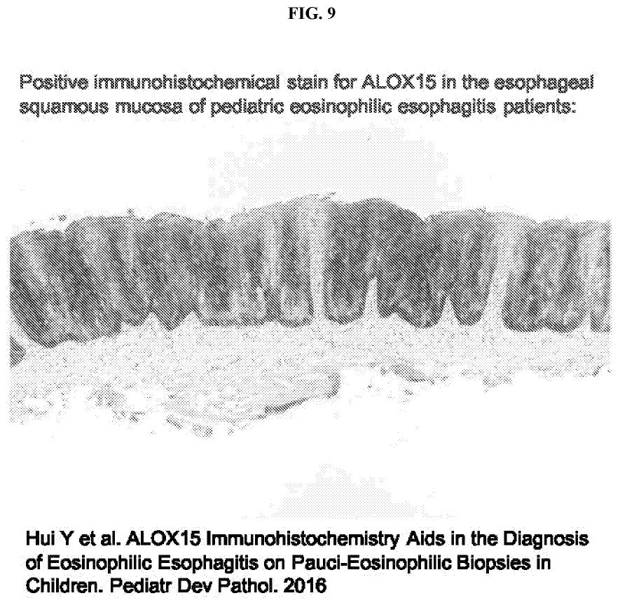

Eosinophilic inflammation can also manifest outside of the airway and Th2-predominant systemic inflammatory process, such as eosinophilic esophagitis, share features with eosinophilic endotypes of airway disease and may benefit from similar therapeutic approaches. As shown in FIG. 9, ALOX15 is highly over-expressed in the esophageal squamous epithelium of pediatric eosinophilic esophagitis patients.

In some embodiments, provided herein are therapies designed to inhibit the production of ALOX15 protein delivered locally to the nasal epithelium, via inhalation to the airway, orally or systemically, that may be efficacious in treating nasal polyposis and related diseases of the upper and lower airway, including chronic rhinosinusitis, asthma, allergic rhinitis, COPD, NSAID-exacerbated respiratory disease, and eosinophilic diseases of the gastro-intestinal tract, such as eosinophilic esophagitis, eosinophilic gastroenteritis and eosinophilic colitis.

Several aspects of this disclosure are described below with reference to example applications for illustration. It should be understood that numerous specific details, relationships, and methods are set forth to provide a full understanding of the composition and methods provided. One having ordinary skill in the relevant art, however, will readily recognize that the embodiments can be practiced without one or more of the specific details and/or with other methods. The disclosure is not limited by the ordering of acts or events, as some acts may occur in different orders and or concurrently with other acts or events. Furthermore, not all illustrated acts or events are required to implement a methodology in accordance with the present disclosure.

All genes, gene names, and gene products disclosed herein are intended to correspond to homologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Thus, for example, for the genes disclosed herein, which in some embodiments relate to mammalian nucleic acid and amino acid sequences are intended to encompass homologous and/or orthologous genes and gene products from other animals including, but not limited to other mammals, fish, amphibians, reptiles, and birds. In an embodiment, the genes or nucleic acid sequences are human.

The terminology used herein is for the purpose of describing particular embodiments only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. In some cases, “about” can mean a range of up to 20%, up to 10%, up to 5%, and up to 1% of a given value. In some cases, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value should be assumed.

In some embodiments, “mRNA” means the presently known mRNA transcript(s) of a targeted gene, and any further transcripts which may be elucidated.

In some embodiments, “antisense oligonucleotides” or “antisense compound” is meant an RNA or DNA molecule that binds to another RNA or DNA (target RNA, DNA). For example, if it is an RNA oligonucleotide it binds to another RNA target by means of RNA-RNA interactions and alters the activity of the target RNA. An antisense oligonucleotide can upregulate or downregulate expression and/or function of a particular polynucleotide. In some cases, an antisense oligonucleotide includes any foreign RNA or DNA molecule which is useful from a therapeutic, diagnostic, or other viewpoint. Such molecules include, for example, antisense RNA or DNA molecules, interference RNA (RNAi), micro RNA, decoy RNA molecules, siRNA, enzymatic RNA, therapeutic editing RNA and agonist and antagonist RNA, antisense oligomeric compounds, antisense oligonucleotides, external guide sequence (EGS) oligonucleotides, alternate splicers, primers, probes, and other oligomeric compounds that hybridize to at least a portion of the target nucleic acid. In some cases, these compounds may be introduced in the form of single-stranded, double-stranded, partially single-stranded, or circular oligomeric compounds.

In some embodiments, the term “oligonucleotide” refers to an oligomer or polymer of ribonucleic acid (RNA) or deoxyribonucleic acid (DNA) or mimetics thereof. The term “oligonucleotide”, also includes linear or circular oligomers of natural and/or modified monomers or linkages, including deoxyribonucleosides, ribonucleosides, substituted and alpha-anomeric forms thereof, peptide nucleic acids (PNA), locked nucleic acids (LNA), phosphorothioate, methylphosphonate, and the like. Oligonucleotides are capable of specifically binding to a target polynucleotide by way of a regular pattern of monomer-to-monomer interactions, such as Watson-Crick type of base pairing, Hoogsteen or reverse Hoogsteen types of base pairing, or the like.

In some embodiments, “dsRNA”, “siRNA”, and “iRNA agent” are used interchangeably to agents that can mediate silencing of a target RNA, e.g., mRNA, e.g., a transcript of a gene that encodes a protein. Such a gene may also be referred to as a target gene. In some cases, the RNA to be silenced is an endogenous gene or a pathogen gene. In addition, RNAs other than mRNA, e.g., tRNAs and viral RNAs, can also be targeted.

The oligonucleotide may be “chimeric”, that is, composed of different regions. “Chimeric” compounds include oligonucleotides, which contain two or more chemical regions, for example, DNA region(s), RNA region(s), PNA region(s) etc. Each chemical region is made up of at least one monomer unit, i.e., a nucleotide in the case of an oligonucleotides compound. These oligonucleotides typically comprise at least one region wherein the oligonucleotide is modified in order to exhibit one or more desired properties. The desired properties of the oligonucleotide include, but are not limited, for example, increased resistance to nuclease degradation, increased cellular uptake, and/or increased binding affinity for the target nucleic acid. Different regions of the oligonucleotide may therefore have different properties. The chimeric oligonucleotides can be formed as mixed structures of two or more oligonucleotides, modified oligonucleotides, oligonucleosides and/or oligonucleotide analogs as described elsewhere herein.

The oligonucleotide can be composed of regions that can be linked in “register”, that is, when the monomers are linked consecutively, as in native DNA, or linked via spacers. The spacers are intended to constitute a covalent “bridge” between the regions and have in some cases a length not exceeding about 100 carbon atoms. The spacers may carry different functionalities, for example, having positive or negative charge, carry special nucleic acid binding properties (intercalators, groove binders, toxins, fluorophores etc.), being lipophilic, inducing special secondary structures like, for example, alanine containing peptides that induce alpha-helices.

In some embodiments, “ALOX15” and “Arachidonate 15-lipoxygenase” are inclusive of all family members, mutants, alleles, fragments, species, coding and noncoding sequences, sense and antisense polynucleotide strands, etc. of ALOX15.

In some embodiments, “ALOX15” and “Arachidonate 15-lipoxygenase”, are considered the same as in the literature and are used interchangeably in the present application.

In some embodiments, “15-hydroxyeicosatetraenoic acid” and “15-HETE” and “15(S)-HETE”, are considered the same as in the literature and are used interchangeably in the present application.

In some embodiments, “Nasal Polyposis” and “Nasal Polyps”, are considered the same as in the literature and are used interchangeably in the present application.

In some embodiments, “Chronic Sinusitis” and “Chronic Rhinosinusitis”, are considered the same as in the literature and are used interchangeably in the present application.

In some embodiments, “oligonucleotide specific for” or “oligonucleotide which targets” refers to an oligonucleotide having a sequence (i) capable of forming a stable complex with a portion of the targeted gene, and/or (ii) capable of forming a stable duplex with a portion of a mRNA transcript of the targeted gene. Stability of the complexes and duplexes can be determined by theoretical calculations and/or in vitro assays.

In some embodiments, “target nucleic acid” encompasses DNA, RNA (e.g., pre-mRNA, mRNA) transcribed from such DNA, and also cDNA derived from such RNA, coding, noncoding sequences, sense, and antisense polynucleotides. The specific hybridization of an oligomeric compound with its target nucleic acid interferes with the normal function of the nucleic acid. This modulation of function of a target nucleic acid by compounds, which specifically hybridize to it, is generally referred to as “antisense”. The functions of DNA to be interfered include, for example, replication and transcription. The functions of RNA to be interfered, include all vital functions such as, for example, translocation of the RNA to the site of protein translation, translation of protein from the RNA, splicing of the RNA to yield one or more mRNA species, and catalytic activity which may be engaged in or facilitated by the RNA. The overall effect of such interference with target nucleic acid function is modulation of the expression of an encoded product or oligonucleotides.

In some embodiments, “enzymatic RNA” is an RNA molecule with enzymatic activity. Enzymatic nucleic acids (ribozymes) act by first binding to a target RNA. Such binding occurs through the target binding portion of an enzymatic nucleic acid which is held in close proximity to an enzymatic portion of the molecule that acts to cleave the target RNA. Thus, the enzymatic nucleic acid first recognizes and then binds a target RNA through base pairing, and once bound to the correct site, acts enzymatically to cut the target RNA.

In some embodiments, “decoy RNA” is an RNA molecule that mimics the natural binding domain for a ligand. The decoy RNA therefore competes with a natural binding target for the binding of a specific ligand. For example, it has been shown that over-expression of HIV trans-activation response (TAR) RNA can act as a “decoy” and efficiently binds HIV tat protein, thereby preventing it from binding to TAR sequences encoded in the HIV RNA. This is meant to be a specific example. Those in the art will recognize that this is but one example, and other embodiments can be readily generated using techniques generally known in the art.

In some embodiments, “monomers” indicates monomers linked by phosphodiester bonds or analogs thereof to form oligonucleotides ranging in size from a few monomeric units, e.g., from about 3-4, to about several hundreds of monomeric units. Analogs of phosphodiester linkages include: phosphorothioate, phosphorodithioate, methylphosphornates, phosphoroselenoate, phosphoramidate, and the like, as more fully described below.

In some embodiments, “nucleotide” includes naturally occurring nucleotides as well as non-naturally occurring nucleotides. It should be clear to the person skilled in the art that various nucleotides which previously have been considered “non-naturally occurring” have subsequently been found in nature. Thus, “nucleotides” includes not only the known purine and pyrimidine heterocycles-containing molecules, but also heterocyclic analogues and tautomers thereof. Illustrative examples of other types of nucleotides are molecules containing adenine, guanine, thymine, cytosine, uracil, purine, xanthine, \hat{ }aminopurine, 8-oxo-N6-memyladenine, 7-deazaxanthine, 7-deazaguanine, N4,N4-ethanocytosin, N6,N6-ethano-2,6-diaminopurine, 5-methyl cytosine, 5-(C3-C6)-alkynylcytosine, 5-fluorouracil, 5-bromouracil, pseudoisocytosine, 2-hydroxy-5-memyl-4-triazolopvridin, isocytosine, isoguanin, inosine and the “non-naturally occurring” nucleotides described in Benner et al, U.S. Pat. No. 5,432,272. The term “nucleotide” is intended to cover every and all of these examples as well as analogues and tautomers thereof. In some cases, nucleotide refers to an adenine, guanine, thymine, cytosine, or uracil, which are considered the naturally occurring nucleotides in relation to therapeutic and diagnostic application in humans. Nucleotides include the natural 2′-deoxy and 2′-hydroxyl sugars, as well as their analogs.

In some embodiments, an “analog” in reference to a nucleotide includes a synthetic nucleotide having modified base moieties and/or modified sugar moieties. Such analogs include synthetic nucleotides designed to enhance binding properties, e.g., duplex or triplex stability, specificity, or the like.

In some embodiments, “hybridization” refers to the pairing of substantially complementary strands of oligomeric compounds. One mechanism of pairing involves hydrogen bonding, which may be Watson-Crick, Hoogsteen or reversed Hoogsteen hydrogen bonding, between complementary nucleoside or nucleotide bases (nucleotides) of the strands of oligomeric compounds. For example, adenine and thymine are complementary nucleotides which pair through the formation of hydrogen bonds. Hybridization can occur under varying circumstances.

In some embodiments, “specifically hybridizable” and “complementary” are terms which are used to indicate a sufficient degree of complementarity such that stable and specific binding occurs between a compound herein and a target RNA molecule.

In some embodiments, an antisense compound is “specifically hybridizable” when binding of the compound to the target nucleic acid interferes with the normal function of the target nucleic acid to cause a modulation of function and/or activity, and there is a sufficient degree of complementarity to avoid non-specific binding of the antisense compound to non-target nucleic acid sequences under conditions in which specific binding is desired, i.e., under physiological conditions in the case of in vivo assays or therapeutic treatment, and under conditions in which assays are performed in the case of in vitro assays.

In some embodiments, “stringent hybridization conditions” or “stringent conditions” refer to conditions under which an oligomeric compound described herein will hybridize to its target sequence, but to a minimal number of other sequences. Stringent conditions are sequence-dependent and will be different in different circumstances. In some cases, “stringent conditions” under which oligomeric compounds hybridize to a target sequence are determined by the nature and composition of the oligomeric compounds and the assays in which they are being investigated. In some examples, stringent hybridization conditions comprise low concentrations (<0.15M) of salts with inorganic cations such as Na+ or K+ (i.e., low ionic strength), temperature higher than 20° C.-25° C. below the Tm of the oligomeric compound-target sequence complex, and the presence of denaturants such as formamide, dimethylformamide, dimethyl sulfoxide, or the detergent sodium dodecyl sulfate (SDS). For example, the hybridization rate decreases 1.1% for each 1% formamide. An example of a high stringency hybridization condition is 0.1× sodium chloride-sodium citrate buffer (SSC)/0.1% (w/v) SDS at 60° C. for 30 minutes.

In some embodiments, specific binding requires a sufficient degree of complementarity to avoid non-specific binding of the oligomeric compound to non-target sequences under conditions in which specific binding is desired, i.e., under physiological conditions in the case of assays or therapeutic treatment, or in the case of in vitro assays, under conditions in which the assays are performed. The non-target sequences may differ by at least 5 nucleotides.

In some embodiments, “complementary,” refers to the capacity for precise pairing between two nucleotides on one or two oligomeric strands. For example, if a nucleobase at a certain position of an antisense compound is capable of hydrogen bonding with a nucleobase at a certain position of a target nucleic acid, said target nucleic acid being a DNA, RNA, or oligonucleotide molecule, then the position of hydrogen bonding between the oligonucleotide and the target nucleic acid is considered to be a complementary position. The oligomeric compound and the further DNA, RNA, or oligonucleotide molecule are complementary to each other when a sufficient number of complementary positions in each molecule are occupied by nucleotides which can hydrogen bond with each other. Thus, “specifically hybridizable” and “complementary” are terms may be used to indicate a sufficient degree of precise pairing or complementarity over a sufficient number of nucleotides such that stable and specific binding occurs between the oligomeric compound and a target nucleic acid.

In some embodiments, a dsRNA agent is “sufficiently complementary” to a target RNA, e.g., a target mRNA, such that the dsRNA agent silences production of protein encoded by the target mRNA. In some embodiments, the dsRNA agent is “exactly complementary” to a target RNA, e.g., the target RNA and the dsRNA duplex agent anneal, for example to form a hybrid made exclusively of Watson-Crick base pairs in the region of exact complementarity. A “sufficiently complementary” target RNA can include an internal region (e.g., of at least 10 nucleotides) that is exactly complementary to a target RNA. Moreover, in some embodiments, the dsRNA agent specifically discriminates a single-nucleotide difference. In some such cases, the dsRNA agent only mediates RNAi if exact complementary is found in the region (e.g., within 7 nucleotides of) the single-nucleotide difference. In some embodiments, an “oligonucleotide” is a nucleic acid molecule (RNA or DNA) having, for example, a length less than 100, 200, 300, or 400 nucleotides.

In some embodiments, “mediates RNAi” refers to the ability to silence, in a sequence specific manner, a target RNA. While not wishing to be bound by theory, it is believed that silencing uses the RNAi machinery or process, and a guide RNA, e.g., a siRNA agent of 21 to 23 nucleotides.

It is understood in the art that the sequence of an oligomeric compound need not be 100% complementary to that of its target nucleic acid to be specifically hybridizable. Moreover, an oligonucleotide may hybridize over one or more segments such that intervening or adjacent segments are not involved in the hybridization event (e.g., a loop structure, mismatch or hairpin structure). In some embodiments, the oligomeric compounds disclosed herein comprise at least about 70%, or at least about 75%, or at least about 80%, or at least about 85%, or at least about 90%, or at least about 95%, or at least about 99% sequence complementarity to a target region within the target nucleic acid sequence to which they are targeted. In a non-limiting example, an antisense compound in which 18 of 20 nucleotides of the anti sense compound are complementary to a target region, and would therefore specifically hybridize, would represent 90 percent complementarity. In this example, the remaining non-complementary nucleotides may be clustered or interspersed with complementary nucleotides and need not be contiguous to each other or to complementary nucleotides. As such, an antisense compound which is 18 nucleotides in length having 4 (four) non-complementary nucleotides which are flanked by two regions of complete complementarity with the target nucleic acid would have 77.8% overall complementarity with the target nucleic acid and would thus fall within the scope of the present disclosure. Percent complementarity of an antisense compound with a region of a target nucleic acid can be determined routinely using BLAST programs (basic local alignment search tools) and PowerBLAST programs known in the art. Percent homology, sequence identity or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman.

In some embodiments, “Thermal Melting Point (Tm)” refers to the temperature, under defined ionic strength, pH, and nucleic acid concentration, at which 50% of the oligonucleotides complementary to the target sequence hybridize to the target sequence at equilibrium. Typically, stringent conditions will be those in which the salt concentration is at least about 0.01 to 1.0 M Na ion concentration (or other salts) at pH 7.0 to 8.3 and the temperature is at least about 30° C. for short oligonucleotides (e.g., 10 to 50 nucleotide). Stringent conditions may also be achieved with the addition of destabilizing agents such as formamide.

In some embodiments, “modulation” means either an increase (stimulation) or a decrease (inhibition) in the expression of a gene.

In some embodiments, “variant”, when used in the context of a polynucleotide sequence, may encompass a polynucleotide sequence related to a wild type gene. This may also include, for example, “allelic,” “splice,” “species,” or “polymorphic” variants. A splice variant may have significant identity to a reference molecule, but will generally have a greater or lesser number of polynucleotides due to alternate splicing of exons during mRNA processing. The corresponding polypeptide may possess additional functional domains or an absence of domains. Species variants are polynucleotide sequences that vary from one species to another. Of particular utility may be variants of wild type gene products. Variants may result from at least one mutation in the nucleic acid sequence and may result in altered mRNAs or in polypeptides whose structure or function may or may not be altered. Any given natural or recombinant gene may have none, one, or many allelic forms. Common mutational changes that give rise to variants are generally ascribed to natural deletions, additions, or substitutions of nucleotides. Each of these types of changes may occur alone, or in combination with the others, one or more times in a given sequence.

In some embodiments, polypeptides generally have significant amino acid identity relative to each other. A polymorphic variant is a variation in the polynucleotide sequence of a particular gene between individuals of a given species. Polymorphic variants also may encompass “single nucleotide polymorphisms” (SNPs,) or single base mutations in which the polynucleotide sequence varies by one base. The presence of SNPs may be indicative of, for example, a certain population with a propensity for a disease state, that is susceptibility versus resistance.

Derivative polynucleotides include nucleic acids subjected to chemical modification, for example, replacement of hydrogen by an alkyl, acyl, or amino group. Derivatives, e.g., derivative oligonucleotides, may comprise non-naturally-occurring portions, such as altered sugar moieties or inter-sugar linkages. Exemplary among these are phosphorothioate and other sulfur containing species which are known in the art. Derivative nucleic acids may also contain labels, including radionucleotides, enzymes, fluorescent agents, chemiluminescent agents, chromogenic agents, substrates, co factors, inhibitors, magnetic particles, and the like.

In some embodiments, a “derivative” polypeptide or peptide is one that is modified, for example, by glycosylation, pegylation, phosphorylation, sulfation, reduction/alkylation, acylation, chemical coupling, or mild formalin treatment. A derivative may also be modified to contain a detectable label, either directly or indirectly, including, but not limited to, a radioisotope, fluorescent, and enzyme label.

In some embodiments, the term “animal” or “patient” includes, for example, any of the following: humans, sheep, elks, deer, mule deer, minks, mammals, monkeys, horses, cattle, pigs, goats, dogs, cats, rats, mice, birds, chicken, reptiles, fish, insects and arachnids.

In some embodiments, “mammal” includes warm blooded mammals that are typically under medical care (e.g., humans and domesticated animals). Examples include feline, canine, equine, bovine, and human, as well as just human.

In some embodiments, “treating” or “treatment” covers the treatment of a disease-state in a mammal, and includes: (a) preventing the disease-state from occurring in a mammal, in particular, when such mammal is predisposed to the disease-state but has not yet been diagnosed as having it; (b) inhibiting the disease-state, e.g., arresting it development; and/or (c) relieving the disease-state, e.g., causing regression of the disease state until a desired endpoint is reached. Treating also includes the amelioration of a symptom of a disease (e.g., lessen the pain or discomfort), wherein such amelioration may or may not be directly affecting the disease (e.g., cause, transmission, expression, etc.).

In some embodiments, “carbohydrate” refers to a compound which is either a carbohydrate per se made up of one or more monosaccharide units having at least 6 carbon atoms (which may be linear, branched or cyclic) with an oxygen, nitrogen or sulfur atom bonded to each carbon atom; or a compound having as a part thereof a carbohydrate moiety made up of one or more monosaccharide units each having at least six carbon atoms (which may be linear, branched or cyclic), with an oxygen, nitrogen or sulfur atom bonded to each carbon atom. Representative carbohydrates include the sugars (mono-, di-, tri- and oligosaccharides containing from about 4-9 monosaccharide units), and polysaccharides such as starches, glycogen, cellulose and polysaccharide gums. Specific monosaccharides include C5 and above (preferably C5-C8) sugars; di- and trisaccharides include sugars having two or three monosaccharide units (preferably C5-C8).

In some embodiments, the term “halo” refers to any radical of fluorine, chlorine, bromine or iodine.

In some embodiments, the term “alkyl” refers to saturated and unsaturated non-aromatic hydrocarbon chains that may be a straight chain or branched chain, containing the indicated number of carbon atoms (these include without limitation propyl, allyl, or propargyl), which may be optionally inserted with N, O, or S. For example, Ci-Cio indicates that the group may have from 1 to 10 (inclusive) carbon atoms in it. The term “alkoxy” refers to an —O-alkyl radical. The term “alkylene” refers to a divalent alkyl (i.e., —R—). The term “alkylenedioxo” refers to a divalent species of the structure -0-R-0-, in which R represents an alkylene. The term “aminoalkyl” refers to an alkyl substituted with an amino. The term “mercapto” refers to an —SH radical. The term “thioalkoxy” refers to an —S-alkyl radical.

In some embodiments, the term “aryl” refers to a 6-carbon monocyclic or 10-carbon bicyclic aromatic ring system wherein 0, 1, 2, 3, or 4 atoms of each ring may be substituted by a substituent. Examples of aryl groups include phenyl, naphthyl and the like. In some embodiments, the term “arylalkyl” or the term “aralkyl” refers to alkyl substituted with an aryl. In some embodiments, the term “arylalkoxy” refers to an alkoxy substituted with aryl.

In some embodiments, the term “cycloalkyl” as employed herein includes saturated and partially unsaturated cyclic hydrocarbon groups having 3 to 12 carbons, for example, 3 to 8 carbons, and, for example, 3 to 6 carbons, wherein the cycloalkyl group additionally may be optionally substituted. Cycloalkyl groups include, without limitation, cyclopropyl, cyclobutyl, cyclopentyl, cyclopentenyl, cyclohexyl, cyclohexenyl, cycloheptyl, and cyclooctyl.

In some embodiments, the term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2, 3, or 4 atoms of each ring may be substituted by a substituent. Examples of heteroaryl groups include pyridyl, furyl or furanyl, imidazolyl, benzimidazolyl, pyrimidinyl, thiophenyl or thienyl, quinolinyl, indolyl, thiazolyl, and the like. The term “heteroarylalkyl” or the term “heteroaralkyl” refers to an alkyl substituted with a heteroaryl. The term “heteroarylalkoxy” refers to an alkoxy substituted with heteroaryl.

In some embodiments, the term “heterocyclyl” refers to a nonaromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent. Examples of heterocyclyl groups include trizolyl, tetrazolyl, piperazinyl, pyrrolidinyl, dioxanyl, morpholinyl, tetrahydrofuranyl, and the like.

In some embodiments, the term “oxo” refers to an oxygen atom, which forms a carbonyl when attached to carbon, an N-oxide when attached to nitrogen, and a sulfoxide or sulfone when attached to sulfur.

In some embodiments, the term “acyl” refers to an alkylcarbonyl, cycloalkylcarbonyl, arylcarbonyl, heterocyclylcarbonyl, or heteroarylcarbonyl substituent, any of which may be further substituted by substituents.

In some embodiments, the term “substituted” refers to the replacement of one or more hydrogen radicals in a given structure with the radical of a specified substituent including, but not limited to: halo, alkyl, alkenyl, alkynyl, aryl, heterocyclyl, thiol, alkylthio, arylthio, alky, thio alkyl, arylthioalkyl, alkylsulfonyl, alkylsulfonylalkyl, aryl sulfonylalkyl, alkoxy, aryloxy, aralkoxy, aminocarbonyl, alkylamino carbonyl, arylaminocarbonyl, alkoxycarbonyl, aryloxycarbonyl, haloalkyl, amino, trifluoromethyl, cyano, nitro, alkylamino, arylamino, alkylaminoalkyl, arylaminoalkyl, aminoalkylamino, hydroxy, alkoxyalkyl, carboxyalkyl, alkoxycarbonylalkyl, aminocarbonyl alkyl, acyl, aralkoxycarbonyl, carboxylic acid, sulfonic acid, sulfonyl, phosphonic acid, aryl, heteroaryl, heterocyclic, and aliphatic. It is understood that the substituent can be further substituted.

### Therapeutic Compositions

In one aspect, provided herein are therapeutic modalities to augment or interfere with a cellular process that cause disease. In some embodiments, a therapeutic modality, composition, or compound described herein may refer to any one of: a dsRNA agent (e.g., siRNA), antisense oligonucleotide, and a small molecule compound. Accordingly, in some cases, therapeutic modality, composition, and compound, may be used interchangeably. In some embodiments, the therapeutic modality, composition, or compound is an oligonucleotide, which may comprise a dsRNA agent or antisense oligonucleotide. In some cases, the therapeutic modality, composition, or compound is a small molecule. In some embodiments, the therapeutic modalities described herein are ALOX15 inhibitors. Non-limiting examples of such therapeutic modalities are shown in Table 1.

TABLE 1
Therapeutic modalities

| Therapy | Description | Target | Mode of Action |
|---|---|---|---|
| Antisense | Single-stranded | mRNA | Prevents translation or splicing |
| Oligonucleotide | DNA or RNA | | |
| (ASO) | | | |
| Small Interfering | Double-stranded | mRNA | Induces innate gene-silencing |
| RNA (siRNA) | RNA | | causing target degradation |
| Anti-Micro RNA | Single-stranded | microRNA | Inactivates microRNA, affecting |
| | RNA | | expression |
| Micro-RNA | Double-stranded | mRNA | Prevents translation |
| Mimic | RNA | | |
| mRNA Analog | Single-stranded | Ribosomes | Induces translation of novel |
| | RNA | | protein |
| Aptamer | Single-stranded | Many | Inactivates or modifies target |
| | DNA or RNA | | |
| CRISPR | gRNA and Cas9 | DNA | Inactivates gene or alters DNA |
| | enzyme | | sequence |
| Small Molecule | Low molecular | Protein | Inhibits a specific function of a |
| Inhibitor | weight organic | | protein or disrupts protein-protein |
| | compound | | interactions |

Antisense compounds have been used to modulate target nucleic acids. In certain instances, such compounds are useful as research tools, diagnostic reagents, and/or as therapeutic agents. Certain DNA-like oligomeric compounds have been shown to reduce protein expression. Certain RNA-like compounds are known to inhibit protein expression in cells. Such RNA-like compounds function, at least in part, through the RNA-inducing silencing complex (RISC). RNA-like compounds may be single-stranded or double-stranded. Antisense compounds have also been shown to alter processing of pre-mRNA and to modulate non-coding RNA molecules. In certain instances antisense compounds have been shown to modulate protein expression by binding to a target messenger RNA (mRNA) encoding the protein. Compositions and methods that facilitate the manufacture, storage, administration, and delivery of such compositions are provided herein.

Many of the therapeutic modalities exemplified in Table 1 have promise to take full-advantage of the major advances brought about by molecular biology. While biochemistry is mainly concerned with how the cell obtains the energy and matter that is required for normal function, molecular biology is mainly concerned with how the cell gets the information to perform its functions. Molecular biology wants to discover the flow of information in the cell. Using the metaphor of computers, the cell is the hardware while the DNA, RNA and proteins are the software. In this sense, in some cases, the purpose of the exemplar modalities is to provide the cell with a new program. The addition of a new cellular function is provided by the insertion of foreign DNA or RNA that lead to the expression of a foreign protein, a native protein at amounts that are not present in the patient, or the suppression of protein expression.

In some embodiments, these modalities deliver polynucleotides to a therapeutically significant percentage of the affected cells in a manner that is both efficient and safe. The delivered polynucleotide can provide, for example, a beneficial function, block activity of an endogenous gene or compensate for a missing endogenous gene. If these materials are appropriately delivered to a patient they can potentially enhance a patient's health and, in some instances, lead to a cure.

Selection of appropriate oligonucleotide modality may be facilitated by using a computer program that automatically aligns nucleic acid sequences and indicates regions of identity or homology. Such programs are used to compare nucleic acid sequences obtained, for example, by searching databases such as GenBank or by sequencing PCR products. Comparison of nucleic acid sequences from a range of species allows the selection of nucleic acid sequences that display an appropriate degree of identity between species. In the case of genes that have not been sequenced, Southern blots are performed to allow a determination of the degree of identity between genes in target species and other species. By performing Southern blots at varying degrees of stringency, as is well known in the art, it is possible to obtain an approximate measure of identity. These procedures allow the selection of oligonucleotides that exhibit a high degree of complementarity to target nucleic acid sequences in a subject to be controlled and a lower degree of complementarity to corresponding nucleic acid sequences in other species. One skilled in the art will realize that there is considerable latitude in selecting appropriate regions of genes.

Double-Stranded RNA

A non-limiting example of a novel therapeutic modality is RNAi. RNA interference or “RNAi” describes the observation that double-stranded RNAi (dsRNA) can block gene expression. Short dsRNA directs gene-specific, post-transcriptional silencing in many organisms, including vertebrates, and has provided a new tool for studying gene function. RNAi is mediated by RNA-induced silencing complex (RISC), a sequence-specific, multi-component nuclease that destroys messenger RNAs homologous to the silencing trigger. RISC is known to contain short RNAs (approximately 22 nucleotides) derived from the double-stranded RNA trigger, but the protein components of this activity remained unknown.

An initial step in RNAi is the activation of RISC, which requires degradation of the sense strand of the dsRNA duplex. Sense strand was known to act as the first RISC substrate that is cleaved by Argonaute 2 in the middle of the duplex region. Immediately after, the cleaved 5′-end and 3′-end fragments of the sense strand are removed from the endonuclease Ago2, and the RISC becomes activated by the antisense strand.

In one aspect of the disclosure, provided is a therapeutic mechanism utilizing RNAi approaches designed to inhibit translation of or degrade ALOX15 mRNA. The RNAi based therapeutic modalities include siRNA targeting of ALOX15 resulting in RISC mediated mRNA cleavage and exonuclease degradation, and/or miRNA targeting resulting in inhibition of translation and/or degradation by exonucleases.

As noted above, RNAi is mediated by dsRNA molecules that have sequence-specific homology to their target nucleic acid sequences. In certain embodiments, the mediators are 5-25 nucleotide “small interfering” RNA duplexes (siRNAs). The siRNAs are derived from the processing of dsRNA by an RNase enzyme known as Dicer. siRNA duplex products are recruited into a multi-protein siRNA complex termed RISC. Without wishing to be bound by any particular theory, a RISC is then believed to be guided to a target nucleic acid (suitably mRNA), where the siRNA duplex interacts in a sequence-specific way to mediate cleavage in a catalytic fashion. Small interfering RNAs that can be used in accordance with the present embodiments can be synthesized and used to inhibitor translation of or degrade ALOX15 mRNA. In some embodiments, small interfering RNAs for use in the methods herein comprise between about 1 to about 50 nucleotides (nt). In examples of non-limiting embodiments, siRNAs can comprise about 5 to about 40 nt, about 5 to about 30 nt, about 10 to about 30 nt, about 15 to about 25 nt, or about 20-25 nucleotides.

In some embodiments, provided are siRNA compounds that are dsRNA agents capable of inhibiting the expression of ALOX15. In some embodiments, the dsRNA agent comprises a sense strand and an antisense strand. In some embodiments, each strand of the dsRNA agent comprises from 12-30 nucleotides in length. In some cases, each strand is between 14-30 nucleotides in length, 17-30 nucleotides in length, 25-30 nucleotides in length, 27-30 nucleotides in length, 17-23 nucleotides in length, 17-21 nucleotides in length, 17-19 nucleotides in length, 19-25 nucleotides in length, 19-23 nucleotides in length, 19-21 nucleotides in length, 21-25 nucleotides in length, or 21-23 nucleotides in length.

In some embodiments, the sense strand and antisense strand form a duplex dsRNA. The duplex region of a dsRNA agent may be 12-30 nucleotide pairs in length. For example, the duplex region can be between 14-30 nucleotide pairs in length, 17-30 nucleotide pairs in length, 25-30 nucleotides in length, 27-30 nucleotide pairs in length, 17-23 nucleotide pairs in length, 17-21 nucleotide pairs in length, 17-19 nucleotide pairs in length, 19-25 nucleotide pairs in length, 19-23 nucleotide pairs in length, 19-21 nucleotide pairs in length, 21-25 nucleotide pairs in length, or 21-23 nucleotide pairs in length. In an example, the duplex region is selected from 15, 16, 17, 18, 19, 20, 21, 22, 23, 24, 25, 26, and 27. In some embodiments, the dsRNA agent comprises a sense strand and an antisense strand, each strand having 14 to 30 nucleotides.

In some embodiments, the dsRNA agent comprises one or more overhang regions and/or capping groups at the 3′-end, or 5′-end, or both ends of a strand. The overhang can be 1-6 nucleotides in length, for instance 2-6 nucleotides in length, 1-5 nucleotides in length, 2-5 nucleotides in length, 1-4 nucleotides in length, 2-4 nucleotides in length, 1-3 nucleotides in length, 2-3 nucleotides in length, or 1-2 nucleotides in length. The overhangs can be the result of one strand being longer than the other, or the result of two strands of the same length being staggered. The overhang can form a mismatch with the target mRNA or it can be complementary to the gene sequences being targeted or can be other sequence. The first and second strands can also be joined, e.g., by additional bases to form a hairpin, or by other non-base linkers.

In some embodiments, the nucleotides in the overhang region of the dsRNA agent are each independently a modified or unmodified nucleotide including, but no limited to 2′-sugar modified, such as, 2-F 2′-Omethyl, thymidine (T), 2′-O-methoxyethyl-5-methyluridine (Teo), 2′-O-methoxyethyladenosine (Aeo), 2′-O-methoxyethyl-5-methylcytidine (m5Ceo), and any combinations thereof. For example, TT can be an overhang sequence for either end on either strand. The overhang can form a mismatch with the target mRNA or it can be complementary to the gene sequences being targeted or can be other sequence.

In some embodiments, the 5′- or 3′-overhangs at the sense strand, antisense strand or both strands of the dsRNA agent may be phosphorylated. In some embodiments, the overhang region contains two nucleotides having a phosphorothioate between the two nucleotides, where the two nucleotides can be the same or different. In some embodiments, the overhang is present at the 3′-end of the sense strand, antisense strand or both strands. In some embodiments, this 3′-overhang is present in the antisense strand. In some embodiments, this 3′-overhang is present in the sense strand.

The dsRNA may comprise a blunt end, located at the 5′-end of the antisense strand (or the 3′-end of the sense strand) or vice versa. In some cases, the antisense strand of the dsRNA has a nucleotide overhang at the 3′-end, and the 5′-end is blunt. While not bound by theory, the asymmetric blunt end at the 5′-end of the antisense strand and 3′-end overhang of the anti sense strand favor the guide strand loading into RISC process. In some embodiments, the dsRNA agent may also have two blunt ends, at both ends of the dsRNA duplex.

In some embodiments, stabilization of synthetic siRNA against rapid nuclease degradation is a prerequisite for in vivo and therapeutic applications. This can be achieved using a variety of stabilization chemistries previously developed for other nucleic acid drugs, such as ribozymes and antisense molecules. These include chemical modifications to the native 2′-OH group in the ribose sugar backbone, such as 2′-O-methyl (2′OMe) and 2′-Fluoro (2′F) substitutions that can be readily introduced into siRNA as 2′-modified nucleotides during RNA synthesis. In some embodiments, chemically stabilized siRNA comprise a 2′OMe, 2′F, 2′-deoxy, or “locked nucleic acid” (LNA). Such modifications can be designed to retain functional RNAi activity. In some embodiments, the modification is tolerated only in certain ill-defined positional or sequence-related contexts. In some embodiments, the introduction of chemical modifications to native siRNA duplexes can have a negative impact on RNAi activity. Therefore, in some cases, the design of chemically modified siRNA may require a stochastic screening approach to identify duplexes that retain potent gene silencing activity.

In some embodiments, inhibition of cleavage of the sense strand impairs the endonucleolytic cleavage of target mRNA. In some embodiments, incorporation of a 2′-0-Me ribose to the Ago2 cleavage site in the sense strand inhibits RNAi in HeLa cells. In some embodiments, for phosphorothioate modifications, cleavage of the sense strand is required for efficient RNAi.

In some embodiments, a siRNA duplex comprises 2′-F modified residues, among other sites and modifications, also at the Ago2 cleavage site, where compatible silencing is achieved as compared to the unmodified siRNAs. In some cases, this modification is not motif specific, e.g., one modification includes 2′-F modifications on all pyrimidines on both sense and antisense strands as long as pyrimidine residue is present. In some embodiments, specific motif modification at the cleavage site of sense strand does not have an effect on gene silencing activity.

In some embodiments, a siRNA duplex comprises two 2′-F modified residues at the Ago2 cleavage site on the sense or antisense strand. In some cases, the modification is sequence specific, e.g., for each particular strand, all pyrimidines or all purines, are modified.

In some embodiments, a siRNA duplex comprises alternative modifications by 2′-OMe or various combinations of 2′-F, 2′-OMe and phosphorothioate modifications to stabilize siRNA in serum. In some cases, the residues at the cleavage site of the antisense strand are not modified with 2′-OMe in order to increase the stability of the siRNA.

In some embodiments, a dsRNA agent or siRNA described herein is modified. In some embodiments, every nucleotide in the sense strand and antisense strand of the dsRNA agent, including the nucleotides that are part of the motifs, is modified. Each nucleotide may be modified with the same or different modification which can include one or more alteration of one or both of the non-linking phosphate oxygens and/or of one or more of the linking phosphate oxygens; alteration of a constituent of the ribose sugar, e.g., of the 2′ hydroxyl on the ribose sugar; wholesale replacement of the phosphate moiety with “dephospho” linkers; modification or replacement of a naturally occurring base; and/or replacement or modification of the ribose-phosphate backbone.

In some embodiments, the modified siRNA comprises modified nucleotides including, but not limited to, 2′OMe nucleotides, 2′-deoxy-2′-fluoro (2′F) nucleotides, 2′-deoxy nucleotides, 2′-O-(2-methoxyethyl) (MOE) nucleotides, locked nucleic acid (LNA) nucleotides, and mixtures thereof. In some embodiments, the modified siRNA comprises 2′OMe nucleotides (e.g., 2′OMe purine and/or pyrimidine nucleotides) such as, for example, 2′OMe-guanosine nucleotides, 2′OMe-uridine nucleotides, 2′OMe-adenosine nucleotides, 2′OMe-cytosine nucleotides, and mixtures thereof. In certain instances, the modified siRNA does not comprise 2′OMe-cytosine nucleotides. In some embodiments, the modified siRNA comprises a hairpin loop structure.

In certain aspects, the modified siRNA has an IC50 less than or equal to ten-fold that of the corresponding unmodified siRNA (e.g., the modified siRNA has an IC50 that is less than or equal to ten-times the IC50 of the corresponding unmodified siRNA). In some embodiments, the modified siRNA has an IC50 less than or equal to three-fold that of the corresponding unmodified siRNA. In some embodiments, the modified siRNA has an IC50 less than or equal to two-fold that of the corresponding unmodified siRNA. A dose response curve can be generated and the IC50 values for the modified siRNA and the corresponding unmodified siRNA can be determined.

In some embodiments, the modified siRNA comprises 3′ overhangs of one, two, three, four, or more nucleotides on one or both sides of the double-stranded region, or may lack overhangs (i.e., have blunt ends). In some cases, the modified siRNA has 3′ overhangs of two nucleotides on each side of the double-stranded region. In certain instances, the 3′ overhang on the antisense strand has complementarity to the target sequence and the 3′ overhang on the sense strand has complementarity to the complementary strand of the target sequence. In certain instances, the 3′ overhangs do not have complementarity to the target sequence or the complementary strand thereof. In some embodiments, the 3′ overhangs comprise one, two, three, four, or more nucleotides such as 2′-deoxy(2′H) nucleotides. In some cases, the 3′ overhangs comprise deoxythymidine (dT) nucleotides.

In some embodiments, the modified siRNA comprises from about 1% to about 100% (e.g., about 1%, 5%, 10%, 15%, 20%, 25%, 30%, 35%, 40%, 45%, 50%, 55%, 60%, 65%, 70%, 75%, 80%, 85%, 90%, 95%, or 100%) modified nucleotides in the double-stranded region of the siRNA duplex. In some embodiments, less than about 30% (e.g., less than about 30%, 25%, 20%, 15%, 10%, or 5%) or from about 1% to about 30% (e.g., from about 1%-30%, 5%-30%, 10%-30%, 15%-30%, 20%-30%, or 25%-30%) of the nucleotides in the double-stranded region comprise modified nucleotides.

In some embodiments, the modified siRNA does not comprise phosphate backbone modifications, e.g., in the sense and/or antisense strand of the double-stranded region. In some embodiments, the modified siRNA does not comprise 2′-deoxy nucleotides, e.g., in the sense and/or antisense strand of the double-stranded region. In certain instances, the nucleotide at the 3′-end of the double-stranded region in the sense and/or antisense strand is not a modified nucleotide. In certain instances, the nucleotides near the 3 ‘-end (e.g., within one, two, three, or four nucleotides of the 3’-end) of the double-stranded region in the sense and/or antisense strand are not modified nucleotides.

As nucleic acids are polymers of subunits, in some cases, a modification occurs at a position which is repeated within a nucleic acid, e.g., a modification of a base, or a phosphate moiety, or a non-linking O of a phosphate moiety. In some cases the modification occurs at all of the subject positions in the nucleic acid. In some cases, the modification does not occur at all of the subject positions in the nucleic acid. By way of a non-limiting, example, a modification may only occur at a 3′ or 5′ terminal position, may only occur in a terminal region, e.g., at a position on a terminal nucleotide or in the last 2, 3, 4, 5, or 10 nucleotides of a strand. A modification may occur in a double strand region, a single strand region, or in both. A modification may occur only in the double strand region of an RNA or may only occur in a single strand region of an RNA. For example, a phosphorothioate modification at a non-linking O position may only occur at one or both termini, may only occur in a terminal region, e.g., at a position on a terminal nucleotide or in the last 2, 3, 4, 5, or 10 nucleotides of a strand, or may occur in double strand and single strand regions, particularly at termini. The 5′ end or ends can be phosphorylated.

In some cases particular bases are included in overhangs, e.g., modified nucleotides or nucleotide surrogates, in single strand overhangs, e.g., in a 5′ or 3′ overhang, or in both. This may enhance stability of the composition. For example, purine nucleotides are included in overhangs. In some embodiments, all or some of the bases in a 3′ or 5′ overhang may be modified, e.g., with a modification described herein. Modifications can include, e.g., the use of modifications at the 2′ position of the ribose sugar, e.g., the use of deoxyribonucleotides, 2′-deoxy-2′-fluoro (2′-F) or 2′-0-methyl modified instead of the ribosugar of the nucleobase, and modifications in the phosphate group, e.g., phosphorothioate modifications. In some cases, overhangs need not be homologous with the target sequence.

In some embodiments, each residue of the sense strand and antisense strand is independently modified with LNA, HNA, CeNA, 2′-methoxyethyl, 2′-O-methyl, 2′-0-allyl, 2′-C-allyl, 2′-deoxy, or 2′-fluoro. The strands can contain more than one modification. In some embodiments, each residue of the sense strand and antisense strand is independently modified with 2′-O-methyl or 2′-fluoro.

In some embodiments, at least two different modifications are present on the sense strand and antisense strand. Those two modifications may be the 2′-O-methyl or 2′-fluoro modifications, or others.

In some embodiments, the sense strand and antisense strand each comprise two differently modified nucleotides selected from 2′-0-methyl or 2′-fluoro.

In some embodiments, each residue of the sense strand and antisense strand is independently modified with 2′-0-methyl nucleotide, 2′-deoxyfluoro nucleotide, 2-O—N-methylacetamido (2′-0-NMA) nucleotide, a 2′-0-dimethylaminoethoxyethyl (2′-0-DMAEOE) nucleotide, 2′-0-aminopropyl (2′-0-AP) nucleotide, or 2′-ara-F nucleotide.

In some embodiments where the dsRNA agent is modified in an alternating motif, the type of modifications contained in the alternating motif may be the same or different. For example, if A, B, C, D each represent one type of modification on the nucleotide, the alternating pattern, i.e., modifications on every other nucleotide, may be the same, but each of the sense strand or antisense strand can be selected from several possibilities of modifications within the alternating motif such as “ABABAB . . . ”, “AC AC AC . . . ” “BDBDBD . . . ” or “CDCDCD . . . ,” etc.

In some embodiments, the dsRNA agent comprises the modification pattern for the alternating motif on the sense strand relative to the modification pattern for the alternating motif on the antisense strand is shifted. The shift may be such that the modified group of nucleotides of the sense strand corresponds to a differently modified group of nucleotides of the anti sense strand and vice versa. For example, the sense strand when paired with the antisense strand in the dsRNA duplex, the alternating motif in the sense strand may start with “ABABAB” from 5′-3′ of the strand and the alternating motif in the antisense strand may start with “BABABA” from 3′-5 of the strand within the duplex region. As another example, the alternating motif in the sense strand may start with “AABBAABB” from 5′-3′ of the strand and the alternating motif in the antisense strand may start with “BBAABBAA” from 3′-5 of the strand within the duplex region, so that there is a complete or partial shift of the modification patterns between the sense strand and the antisense strand.

In some embodiments, the dsRNA agent comprises the pattern of the alternating motif of 2′-0-methyl modification and 2′-F modification on the sense strand initially has a shift relative to the pattern of the alternating motif of 2′-0-methyl modification and 2′-F modification on the antisense strand initially, i.e., the 2′-0-methyl modified nucleotide on the sense strand base pairs with a 2′-F modified nucleotide on the antisense strand and vice versa. The 1 position of the sense strand may start with the 2′-F modification, and the 1 position of the antisense strand may start with the 2′-O-methyl modification. The introduction of one or more motifs of three identical modifications on three consecutive nucleotides to the sense strand and/or antisense strand interrupts the initial modification pattern present in the sense strand and/or antisense strand. This interruption of the modification pattern of the sense and/or antisense strand by introducing one or more motifs of three identical modifications on three consecutive nucleotides to the sense and/or anti sense strand may enhance the gene silencing activity to the target gene.

The dsRNA agent may comprise at least one phosphorothioate or methylphosphonate internucleotide linkage. The phosphorothioate or methylphosphonate internucleotide linkage modification may occur on any nucleotide of the sense strand or antisense strand or both in any position of the strand. For instance, the internucleotide linkage modification may occur on every nucleotide on the sense strand and/or antisense strand; each internucleotide linkage modification may occur in an alternating pattern on the sense strand or antisense strand; or the sense strand or antisense strand comprises both internucleotide linkage modifications in an alternating pattern. The alternating pattern of the internucleotide linkage modification on the sense strand may be the same or different from the antisense strand, and the alternating pattern of the internucleotide linkage modification on the sense strand may have a shift relative to the alternating pattern of the internucleotide linkage modification on the antisense strand.

In some embodiments, the dsRNA comprises the phosphorothioate or methylphosphonate internucleotide linkage modification in the overhang region. For example, the overhang region comprises two nucleotides having a phosphorothioate or methylphosphonate internucleotide linkage between the two nucleotides. Internucleotide linkage modifications also may be made to link the overhang nucleotides with the terminal paired nucleotides within duplex region. For example, at least 2, 3, 4, or all the overhang nucleotides may be linked through phosphorothioate or methylphosphonate internucleotide linkage, and optionally, there may be additional phosphorothioate or methylphosphonate internucleotide linkages linking the overhang nucleotide with a paired nucleotide that is next to the overhang nucleotide. For instance, there may be at least two phosphorothioate internucleotide linkages between the terminal three nucleotides, in which two of the three nucleotides are overhang nucleotides, and the third is a paired nucleotide next to the overhang nucleotide. In some cases, these terminal three nucleotides may be at the 3′-end of the antisense strand.

In some embodiments, the sense strand of the dsRNA comprises 1-10 blocks of two to ten phosphorothioate or methylphosphonate internucleotide linkages separated by 1, 2, 3, 4, 5, 6, 7, 8, 9, 10, 11, 12, 13, 14, 15 or 16 phosphate internucleotide linkages, wherein one of the phosphorothioate or methylphosphonate internucleotide linkages is placed at any position in the oligonucleotide sequence and the said sense strand is paired with an antisense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA comprises two blocks of two phosphorothioate or methylphosphonate internucleotide linkages separated by 1, 2, 3, 4, 5, 6, 7, 8, 9, 10, 11, 12, 13, 14, 15, 16, 17, or 18 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA comprises two blocks of three phosphorothioate or methylphosphonate internucleotide linkages separated by 1, 2, 3, 4, 5, 6, 7, 8, 9, 10, 11, 12, 13, 14, 15 or 16 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA comprises two blocks of four phosphorothioate or methylphosphonate internucleotide linkages separated by 1, 2, 3, 4, 5, 6, 7, 8, 9, 10, 11, 12, 13 or 14 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA comprises two blocks of five phosphorothioate or methylphosphonate internucleotide linkages separated by 1, 2, 3, 4, 5, 6, 7, 8, 9, 10, 11 or 12 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA comprises two blocks of six phosphorothioate or methylphosphonate internucleotide linkages separated by 1, 2, 3, 4, 5, 6, 7, 8, 9 or 10 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA comprises two blocks of seven phosphorothioate or methylphosphonate internucleotide linkages separated by 1, 2, 3, 4, 5, 6, 7 or 8 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA comprises two blocks of eight phosphorothioate or methylphosphonate internucleotide linkages separated by 1, 2, 3, 4, 5 or 6 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA comprises two blocks of nine phosphorothioate or methylphosphonate internucleotide linkages separated by 1, 2, 3 or 4 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the dsRNA comprises one or more phosphorothioate or methylphosphonate internucleotide linkage modification within 1-10 of the termini position(s) of the sense and/or antisense strand. For example, at least 2, 3, 4, 5, 6, 7, 8, 9 or 10 nucleotides may be linked through phosphorothioate or methylphosphonate internucleotide linkage at one end or both ends of the sense and/or antisense strand.

In some embodiments, the dsRNA comprises one or more phosphorothioate or methylphosphonate internucleotide linkage modification within 1-10 of the internal region of the duplex of each of the sense and/or antisense strand. For example, at least 2, 3, 4, 5, 6, 7, 8, 9 or 10 nucleotides may be linked through phosphorothioate methylphosphonate internucleotide linkage at position 8-16 of the duplex region counting from the 5′-end of the sense strand; the dsRNA can optionally further comprise one or more phosphorothioate or methylphosphonate internucleotide linkage modification within 1-10 of the termini position(s).

In some embodiments, the dsRNA comprises one to five phosphorothioate or methylphosphonate internucleotide linkage modification(s) within position 1-5 and one to five phosphorothioate or methylphosphonate internucleotide linkage modification(s) within position 18-23 of the sense strand (counting from the 5′-end), and one to five phosphorothioate or methylphosphonate internucleotide linkage modification at positions 1 and 2 and one to five within positions 18-23 of the antisense strand (counting from the 5′-end).

In some embodiments, the dsRNA comprises one phosphorothioate internucleotide linkage modification within position 1-5 and one phosphorothioate or methylphosphonate internucleotide linkage modification within position 18-23 of the sense strand (counting from the 5′-end), and one phosphorothioate internucleotide linkage modification at positions 1 and 2 and two phosphorothioate or methylphosphonate internucleotide linkage modifications within positions 18-23 of the antisense strand (counting from the 5′-end).

In some embodiments, the dsRNA comprises two phosphorothioate internucleotide linkage modifications within position 1-5 and one phosphorothioate internucleotide linkage modification within position 18-23 of the sense strand (counting from the 5′-end), and one phosphorothioate internucleotide linkage modification at positions 1 and 2 and two phosphorothioate internucleotide linkage modifications within positions 18-23 of the antisense strand (counting from the 5′-end).

In some embodiments, the dsRNA comprises two phosphorothioate internucleotide linkage modifications within position 1-5 and two phosphorothioate internucleotide linkage modifications within position 18-23 of the sense strand (counting from the 5′-end), and one phosphorothioate internucleotide linkage modification at positions 1 and 2 and two phosphorothioate internucleotide linkage modifications within positions 18-23 of the antisense strand (counting from the 5′-end). In some embodiments, the dsRNA further comprises two phosphorothioate internucleotide linkage modifications within position 1-5 and two phosphorothioate internucleotide linkage modifications within position 18-23 of the sense strand (counting from the 5′-end), and one phosphorothioate internucleotide linkage modification at positions 1 and 2 and one phosphorothioate internucleotide linkage modification within positions 18-23 of the antisense strand (counting from the 5′-end).

In some embodiments, the dsRNA comprises one phosphorothioate internucleotide linkage modification within position 1-5 and one phosphorothioate internucleotide linkage modification within position 18-23 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end).

In some embodiments, the dsRNA comprises one phosphorothioate internucleotide linkage modification within position 1-5 and one within position 18-23 of the sense strand (counting from the 5′-end), and two phosphorothioate internucleotide linkage modification at positions 1 and 2 and one phosphorothioate internucleotide linkage modification within positions 18-23 of the antisense strand (counting from the 5′-end).

In some embodiments, the dsRNA comprises one phosphorothioate internucleotide linkage modification within position 1-5 (counting from the 5′-end), and two phosphorothioate internucleotide linkage modifications at positions 1 and 2 and one phosphorothioate internucleotide linkage modification within positions 18-23 of the antisense strand (counting from the 5′-end).

In some embodiments, the dsRNA comprises two phosphorothioate internucleotide linkage modifications within position 1-5 (counting from the 5′-end), and one phosphorothioate internucleotide linkage modification at positions 1 and 2 and two phosphorothioate internucleotide linkage modifications within positions 18-23 of the antisense strand (counting from the 5′-end).

In some embodiments, the dsRNA comprises two phosphorothioate internucleotide linkage modifications within position 1-5 and one within position 18-23 of the sense strand (counting from the 5′-end), and two phosphorothioate internucleotide linkage modifications at positions 1 and 2 and one phosphorothioate internucleotide linkage modification within positions 18-23 of the antisense strand (counting from the 5′-end).

In some embodiments, the dsRNA comprises two phosphorothioate internucleotide linkage modifications within position 1-5 and one phosphorothioate internucleotide linkage modification within position 18-23 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end).

In some embodiments, the dsRNA comprises two phosphorothioate internucleotide linkage modifications within position 1-5 and one phosphorothioate internucleotide linkage modification within position 18-23 of the sense strand (counting from the 5′-end), and one phosphorothioate internucleotide linkage modification at positions 1 and 2 and two phosphorothioate internucleotide linkage modifications within positions 18-23 of the antisense strand (counting from the 5′-end).

In some embodiments, the dsRNA comprises two phosphorothioate internucleotide linkage modifications at position 1 and 2, and two phosphorothioate internucleotide linkage modifications at position 20 and 21 of the sense strand (counting from the 5′-end), and one phosphorothioate internucleotide linkage modification at positions 1 and one at position 21 of the antisense strand (counting from the 5′-end).

In some embodiments, the dsRNA comprises one phosphorothioate internucleotide linkage modification at position 1, and one phosphorothioate internucleotide linkage modification at position 21 of the sense strand (counting from the 5′-end), and two phosphorothioate internucleotide linkage modifications at positions 1 and 2 and two phosphorothioate internucleotide linkage modifications at positions 20 and 21 the antisense strand (counting from the 5′-end).

In some embodiments, the dsRNA comprises two phosphorothioate internucleotide linkage modifications at position 1 and 2, and two phosphorothioate internucleotide linkage modifications at position 21 and 22 of the sense strand (counting from the 5′-end), and one phosphorothioate internucleotide linkage modification at positions 1 and one phosphorothioate internucleotide linkage modification at position 21 of the antisense strand (counting from the 5′-end).

In some embodiments, the dsRNA comprises one phosphorothioate internucleotide linkage modification at position 1, and one phosphorothioate internucleotide linkage modification at position 21 of the sense strand (counting from the 5′-end), and two phosphorothioate internucleotide linkage modifications at positions 1 and 2 and two phosphorothioate internucleotide linkage modifications at positions 21 and 22 the antisense strand (counting from the 5′-end).

In some embodiments, the dsRNA comprises two phosphorothioate internucleotide linkage modifications at position 1 and 2, and two phosphorothioate internucleotide linkage modifications at position 22 and 23 of the sense strand (counting from the 5′-end), and one phosphorothioate internucleotide linkage modification at positions 1 and one phosphorothioate internucleotide linkage modification at position 21 of the antisense strand (counting from the 5′-end).

In some embodiments, the dsRNA comprises one phosphorothioate internucleotide linkage modification at position 1, and one phosphorothioate internucleotide linkage modification at position 21 of the sense strand (counting from the 5′-end), and two phosphorothioate internucleotide linkage modifications at positions 1 and 2 and two phosphorothioate internucleotide linkage modifications at positions 23 and 23 the antisense strand (counting from the 5′-end).

In some embodiments, the dsRNA agent comprises mismatch(es) with the target, within the duplex, or combinations thereof. The mismatch can occur in the overhang region or the duplex region. The base pair can be ranked on the basis of their propensity to promote dissociation or melting (e.g., on the free energy of association or dissociation of a particular pairing, the simplest approach is to examine the pairs on an individual pair basis, though next neighbor or similar analysis can also be used). In terms of promoting dissociation, in some cases: A:U may be preferred over G:C; G:U may be preferred over G:C; and I:C may be preferred over G:C (I=inosine). In some cases, mismatches, e.g., non-canonical or other than canonical pairings (as described elsewhere herein) may be preferred over canonical (A:T, A:U, G:C) pairings; and pairings which include a universal base may be preferred over canonical pairings. In some embodiments, the dsRNA agent comprises at least one of the first 1, 2, 3, 4, or 5 base pairs within the duplex regions from the 5′-end of the antisense strand, which can be chosen independently from the group of: A:U, G:U, I:C, and mismatched pairs, e.g., non-canonical or other than canonical pairings or pairings which include a universal base, to promote the dissociation of the antisense strand at the 5′-end of the duplex.

In some embodiments, the nucleotide at the 1 position within the duplex region from the 5′-end in the antisense strand is selected from the group consisting of A, dA, dU, U, and dT. In some embodiments, at least one of the first 1, 2 or 3 base pair within the duplex region from the 5′-end of the antisense strand is an AU base pair. For example, the first base pair within the duplex region from the 5′-end of the antisense strand is an AU base pair.

In some embodiments, the dsRNA agent comprises a conjugation of one or more carbohydrate moieties. In some cases, this may optimize one or more properties of the dsRNA agent. In some cases, the carbohydrate moiety will be attached to a modified subunit of the dsRNA agent. For example, the ribose sugar of one or more ribonucleotide subunits of a dsRNA agent can be replaced with another moiety, e.g., a non-carbohydrate (preferably cyclic) carrier to which is attached a carbohydrate ligand. In some cases, a ribonucleotide subunit in which the ribose sugar of the subunit has been so replaced is referred to herein as a ribose replacement modification subunit (RRMS). A cyclic carrier may be a carbocyclic ring system, i.e., all ring atoms are carbon atoms, or a heterocyclic ring system, i.e., one or more ring atoms may be a heteroatom, e.g., nitrogen, oxygen, sulfur. The cyclic carrier may be a monocyclic ring system, or may contain two or more rings, e.g. fused rings. The cyclic carrier may be a fully saturated ring system, or it may contain one or more double bonds.

In some embodiments, the dsRNA is conjugated or otherwise connected to a ligand. The ligand may be attached to the polynucleotide via a carrier. Non-limiting examples of carriers include (i) at least one “backbone attachment point,” e.g., two “backbone attachment points” and (ii) at least one “tethering attachment point.” In some cases, a “backbone attachment point” is a functional group, e.g. a hydroxyl group, or generally, a bond available for, and that is suitable for incorporation of, the carrier into the backbone, e.g., the phosphate, or modified phosphate, e.g., sulfur containing, backbone, of a ribonucleic acid. A “tethering attachment point” (TAP) in some embodiments refers to a constituent ring atom of the cyclic carrier, e.g., a carbon atom or a heteroatom (distinct from an atom which provides a backbone attachment point), that connects a selected moiety. The moiety can be, e.g., a carbohydrate, e.g. monosaccharide, disaccharide, trisaccharide, tetrasaccharide, oligosaccharide and polysaccharide.

In some embodiments, the selected moiety is connected by an intervening tether to the cyclic carrier. In some cases, the cyclic carrier comprises a functional group, e.g., an amino group, or generally, provides a bond that is suitable for incorporation or tethering of another chemical entity, e.g., a ligand to the constituent ring.

In some embodiments, the dsRNA is conjugated to a ligand via a carrier, wherein the carrier can be cyclic group or acyclic group; the cyclic group is selected from pyrrolidinyl, pyrazolinyl, pyrazolidinyl, imidazolinyl, imidazolidinyl, piperidinyl, piperazinyl, [1,3]dioxolane, oxazolidinyl, isoxazolidinyl, morpholinyl, thiazolidinyl, isothiazolidinyl, quinoxalinyl, pyridazinonyl, tetrahydrofuryl and decalin; the acyclic group is selected from serinol backbone or diethanolamine backbone. The dsRNA agent may be conjugated to one or more ligands. The ligand can be attached to the sense strand, antisense strand or both strands, at the 3′-end, 5′-end or both ends. For instance, the ligand may be conjugated to the sense strand, in particular, the 3′-end of the sense strand.

In some embodiments, provided herein is a dsRNA agent capable of inhibiting the expression of a target gene. In some cases, the dsRNA agent comprises a sense strand and an antisense strand, each strand having 14 to 30 nucleotides. In some cases, every nucleotide in the sense strand and antisense strand has been modified. In some cases, the modifications on sense strand and antisense strand each independently comprises at least two different modifications.

In some embodiments, provided is a dsRNA agent capable of inhibiting the expression of a target gene. In some cases, the dsRNA agent comprises a sense strand and an antisense strand, each strand having 14 to 30 nucleotides. In some cases, the sense strand contains at least one motif of three identical modifications on three consecutive nucleotides, where at least one of the motifs occurs at or near the cleavage site in the antisense strand. In some cases, the antisense strand contains at least one motif of three identical modifications on three consecutive nucleotides. In some cases, the modification pattern of the antisense strand is shifted by one or more nucleotides relative to the modification pattern of the sense strand.

In some embodiments, provided is a dsRNA agent capable of inhibiting the expression of a target gene. In some cases, the dsRNA agent comprises a sense strand and an antisense strand, each strand having 14 to 30 nucleotides. In some cases, the sense strand contains at least two motifs of three identical modifications on three consecutive nucleotides, when at least one of the motifs occurs at the cleavage site in the strand and at least one of the motifs occurs at another portion of the strand that is separated from the motif at the cleavage site by at least one nucleotide. In some cases, the antisense strand contains at least one motif of three identical modifications on three consecutive nucleotides, where at least one of the motifs occurs at or near the cleavage site in the strand and at least one of the motifs occurs at another portion of the strand that is separated from the motif at or near cleavage site by at least one nucleotide.

In some embodiments, provided is a dsRNA agent capable of inhibiting the expression of a target gene. In some cases, the dsRNA agent comprises a sense strand and an antisense strand, each strand having 14 to 30 nucleotides. In some cases, the sense strand contains at least two motifs of three identical modifications on three consecutive nucleotides, where at least one of the motifs occurs at the cleavage site in the strand and at least one of the motifs occurs at another portion of the strand that is separated from the motif at the cleavage site by at least one nucleotide. In some cases, the antisense strand contains at least one motif of three identical modifications on three consecutive nucleotides, where at least one of the motifs occurs at or near the cleavage site in the strand and at least one of the motifs occurs at another portion of the strand that is separated from the motif at or near cleavage site by at least one nucleotide. In some cases, the modification in the motif occurring at the cleavage site in the sense strand is different than the modification in the motif occurring at or near the cleavage site in the antisense strand.

In some embodiments, provided is a dsRNA agent capable of inhibiting the expression of a target gene. In some cases, the dsRNA agent comprises a sense strand and an antisense strand, each strand having 12 to 30 nucleotides. In some cases, the sense strand contains at least one motif of three 2′-F modifications on three consecutive nucleotides, where at least one of the motifs occurs at the cleavage site in the strand. In some cases, the antisense strand contains at least one motif of three 2′-0-methyl modifications on three consecutive nucleotides.

In some embodiments, the sense strand may further comprises one or more motifs of three identical modifications on three consecutive nucleotides, where the one or more additional motifs occur at another portion of the strand that is separated from the three 2′-F modifications at the cleavage site by at least one nucleotide. The antisense strand may further comprises one or more motifs of three identical modifications on three consecutive nucleotides, where the one or more additional motifs occur at another portion of the strand that is separated from the three 2′-0-methyl modifications by at least one nucleotide. In some cases, at least one of the nucleotides having a 2′-F modification may form a base pair with one of the nucleotides having a 2′-0-methyl modification.

Antisense Oligonucleotides

DNA-RNA and RNA-RNA hybridization are important to many aspects of nucleic acid function, including DNA replication, transcription, and translation. Hybridization is also central to a variety of technologies that either detect a particular nucleic acid or alter its expression. Antisense nucleotides, for example, disrupt gene expression by hybridizing to target RNA, thereby interfering with RNA splicing, transcription, translation, and replication. Antisense DNA has the added feature that DNA-RNA hybrids serve as a substrate for digestion by ribonuclease H, an activity that is present in most cell types. Antisense molecules can be delivered into cells, as is the case for oligodeoxynucleotides (ODNs), or they can be expressed from endogenous genes as RNA molecules. The FDA recently approved an antisense drug, KYNAMRO® (for treatment of homozygous familial hypercholesterolemia), reflecting that antisense molecules have therapeutic utility.

In some embodiments, provided are methods for inhibiting the action of a natural transcript by using antisense oligonucleotide(s) targeted to any region of the natural transcript. It is also contemplated herein that inhibition of the natural transcript can be achieved by siRNA, ribozymes and small molecules, which are considered to be within the scope of this disclosure.

In some embodiments, provided is a method of modulating function and/or expression of an ALOX15 polynucleotide in mammalian cells or tissues in vivo or in vitro comprising contacting said cells or tissues with an antisense oligonucleotide 5 to 30 nucleotides in length, wherein said oligonucleotide has at least 50% sequence identity to a reverse complement of a polynucleotide comprising 5 to 30 consecutive nucleotides within nucleotides 1 to 2715 of SEQ ID NO: 2, and any variants, alleles, homologs, mutants, derivatives, fragments and complementary sequences thereby modulating function and/or expression of the ALOX15 polynucleotide in mammalian cells or tissues in vivo or in vitro.

In some embodiments, an oligonucleotide targets a natural sequence of ALOX15 polynucleotides, for example, nucleotides set forth in SEQ ID NO: 1, and any variants, alleles, homologs, mutants, derivatives, fragments and complementary sequences thereto.

In some embodiments, a composition comprises one or more antisense oligonucleotides which bind to sense ALOX15 polynucleotides. In some embodiments, the oligonucleotides comprise one or more modified or substituted nucleotides. In some embodiments, the oligonucleotides comprise one or more modified bonds. In some embodiments, the modified nucleotides comprise modified bases comprising phosphorothioate, methylphosphonate, peptide nucleic acids, 2′-0-methyl, methoxyethyl, fluoro- or carbon, methylene or other locked nucleic acid (LNA) molecules. In some embodiments, the modified nucleotides are locked nucleic acid molecules, including a-L-LNA. In some embodiments, the oligonucleotides are administered to a patient intranasally, subcutaneously, intramuscularly, intravenously or intraperitoneally. In some embodiments, the oligonucleotides are administered in a pharmaceutical composition. In some exemplary embodiments, a treatment regimen comprises administering the antisense compounds at least once to patient; however, this treatment can be modified to include multiple doses over a period of time. The treatment can be combined with one or more other types of therapies. In some embodiments, the oligonucleotides are encapsulated in a liposome or attached to a carrier molecule (e.g. cholesterol, TAT peptide).

In some embodiments, antisense oligonucleotides are used to prevent or treat diseases or disorders associated with ALOX15 family members. Exemplary Arachidonate 15-lipoxygenase (ALOX15) mediated diseases and disorders which can be treated with cell/tissues regenerated from stem cells obtained using the antisense compounds comprise: a disease or disorder associated with abnormal function and/or expression of ALOX15, an inflammatory disease or a neo-proliferative disease such as chronic sinusitis or chronic sinusitis with nasal polyps.

In some embodiments, modulation of ALOX15 by one or more antisense oligonucleotides is achieved by administering an antisense composition to a patient in need thereof, to prevent or treat any disease or disorder related to ALOX15 abnormal expression, function, or activity, as compared to a normal control.

In some embodiments, the oligonucleotides are specific for polynucleotides of ALOX15, which includes, without limitation noncoding regions. The ALOX15 targets comprise variants of ALOX15; mutants of ALOX15, including SNPs; noncoding sequences of ALOX15; alleles, fragments and the like. In some cases, the oligonucleotide is an antisense RNA molecule.

The target nucleic acid molecule described herein is not limited to ALOX15 polynucleotides alone, but extends to any of the isoforms, receptors, homologs, non-coding regions and the like of ALOX15.

In some embodiments, an oligonucleotide targets a natural antisense sequence (natural antisense to the coding and non-coding regions) of ALOX15 targets, including, without limitation, variants, alleles, homologs, mutants, derivatives, fragments and complementary sequences thereto. In some cases, the oligonucleotide is an antisense RNA or DNA molecule.

In some embodiments, the oligomeric compounds described herein include variants in which a different base is present at one or more of the nucleotide positions in the compound. For example, if the first nucleotide is an adenine, variants may be produced which contain thymidine, guanosine, cytidine or other natural or unnatural nucleotides at this position. This may be done at any of the positions of the antisense compound. These compounds are then tested using the methods described herein to determine their ability to inhibit expression of a target nucleic acid.

In some embodiments, homology, sequence identity or complementarity, between the antisense compound and target is from about 50% to about 60%. In some embodiments, homology, sequence identity or complementarity, is from about 60% to about 70%. In some embodiments, homology, sequence identity or complementarity, is from about 70% to about 80%. In some embodiments, homology, sequence identity or complementarity, is from about 80% to about 90%. In some embodiments, homology, sequence identity or complementarity, is about 90%, about 92%, about 94%, about 95%, about 96%, about 97%, about 98%, about 99% or about 100%.

In some embodiments, an antisense compound is specifically hybridizable when binding of the compound to the target nucleic acid interferes with the normal function of the target nucleic acid to cause a loss of activity, and there is a sufficient degree of complementarity to avoid non-specific binding of the antisense compound to non-target nucleic acid sequences under conditions in which specific binding is desired. Such conditions include, e.g., physiological conditions in the case of in vivo assays or therapeutic treatment, and conditions in which assays are performed in the case of in vitro assays.

In some embodiments, an antisense compound, whether DNA, RNA, chimeric, substituted etc, is specifically hybridizable when binding of the compound to the target DNA or RNA molecule interferes with the normal function of the target DNA or RNA to cause a loss of utility, and there is a sufficient degree of complementarily to avoid non-specific binding of the antisense compound to non-target sequences under conditions in which specific binding is desired, e.g., under physiological conditions in the case of in vivo assays or therapeutic treatment, and in the case of in vitro assays, under conditions in which the assays are performed.

In some embodiments, targeting of ALOX15 includes without limitation, antisense sequences which are identified and expanded, using for example, PCR, hybridization etc., based on the sequence set forth as SEQ ID NO: 2, and the like, to modulate the expression or function of ALOX15. In some embodiments, expression or function is down-regulated as compared to a control.

In some embodiments, oligonucleotides comprise nucleic acid sequences set forth as SEQ ID NOS: 3 and/or 5, including antisense sequences which are identified and expanded, using for example, PCR, hybridization etc. These oligonucleotides can comprise one or more modified nucleotides, shorter or longer fragments, modified bonds and the like. Examples of modified bonds or internucleotide linkages comprise phosphorothioate, phosphorodithioate or the like. In some embodiments, the nucleotides comprise a phosphorus derivative. The phosphorus derivative (or modified phosphate group) which may be attached to the sugar or sugar analog moiety in the modified oligonucleotides, may be a monophosphate, diphosphate, triphosphate, alkylphosphate, alkanephosphate, phosphorothioate or the like.

Antisense oligonucleotides have been employed as therapeutic moieties in the treatment of disease states in animals and man. Antisense oligonucleotides have been safely and effectively administered to humans and numerous clinical trials are presently underway. It is thus established that oligonucleotides can be useful therapeutic modalities that can be configured to be useful in treatment regimens for treatment of cells, tissues and animals, especially humans.

In some embodiments, oligomeric antisense compounds, particularly oligonucleotides, bind to target nucleic acid molecules and modulate the expression and/or function of molecules encoded by a target gene. The functions of DNA to be interfered with comprise, for example, replication and transcription. The functions of RNA to be interfered with comprise all vital functions such as, for example, translocation of the RNA to the site of protein translation, translation of protein from the RNA, splicing of the RNA to yield one or more mRNA species, and catalytic activity which may be engaged in or facilitated by the RNA. The functions may be up-regulated or inhibited depending on the functions desired.

The antisense compounds include antisense oligomeric compounds, antisense oligonucleotides, external guide sequence (EGS) oligonucleotides, alternate splicers, primers, probes, and other oligomeric compounds that hybridize to at least a portion of the target nucleic acid. As such, these compounds may be introduced in the form of single-stranded, double-stranded, partially single-stranded, or circular oligomeric compounds.

Targeting an antisense compound to a particular nucleic acid molecule can be a multistep process. In some cases, the process begins with the identification of a target nucleic acid whose function is to be modulated. This target nucleic acid may be, for example, a cellular gene (or mRNA transcribed from the gene) whose expression is associated with a particular disorder or disease state, or a nucleic acid molecule from an infectious agent. In some embodiments, the target nucleic acid encodes Arachidonate 15-lipoxygenase (ALOX15).

The targeting process may include determination of at least one target region, segment, or site within the target nucleic acid for the antisense interaction to occur such that the desired effect, e.g., modulation of expression, will result. In some embodiments, “region” is a portion of the target nucleic acid having at least one identifiable structure, function, or characteristic. Within regions of target nucleic acids are segments. In some embodiments, “segments” are smaller or sub-portions of regions within a target nucleic acid. In some embodiments, “sites,” are positions within a target nucleic acid.

In some embodiments, the antisense oligonucleotides bind to the natural antisense sequences of Arachidonate 15-lipoxygenase (ALOX15) and modulate the expression and/or function of ALOX15 (SEQ ID NO: 1). Examples of antisense sequences include SEQ ID NOS: 3 and 5.

In some embodiments, the antisense oligonucleotides bind to one or more segments of Arachidonate 15-lipoxygenase (ALOX15) polynucleotides and modulate the expression and/or function of ALOX15. In some cases, the segments comprise at least five consecutive nucleotides of the ALOX15 sense or antisense polynucleotides.

In some embodiments, oligonucleotides comprise one or more modified nucleotides, shorter or longer fragments, modified bonds and the like. Examples of modified bonds or internucleotide linkages comprise phosphorothioate, phosphorodithioate and the like. In some embodiments, the nucleotides comprise a phosphorus derivative. The phosphorus derivative (or modified phosphate group) which may be attached to the sugar or sugar analog moiety in the modified oligonucleotides may be a monophosphate, diphosphate, triphosphate, alkylphosphate, alkanephosphate, phosphorothioate or the like.

In some embodiments, the translation initiation codon is 5′-AUG (in transcribed mRNA molecules; 5-ATG in the corresponding DNA molecule), and the translation initiation codon is also referred to as the “AUG codon,” the “start codon” or the “AUG start codon”. A minority of genes have a translation initiation codon having the RNA sequence 5′-GUG, 5′-UUG or 5′-CUG; and 5′-AUA, 5′-ACG and 5′-CUG have been shown to function in vivo. Thus, in some cases, the terms “translation initiation codon” and “start codon” can encompass many codon sequences, even though the initiator amino acid in each instance is typically methionine (in eukaryotes) or formylmethionine (in prokaryotes). Eukaryotic and prokaryotic genes may have two or more alternative start codons, any one of which may be utilized for translation initiation in a particular cell type or tissue, or under a particular set of conditions. In some embodiments, “start codon” and “translation initiation codon” refer to the codon or codons that are used in vivo to initiate translation of an mRNA transcribed from a gene encoding Arachidonate 15-lipoxygenase, (ALOX15), regardless of the sequence(s) of such codons. A translation termination codon (or “stop codon”) of a gene may have one of three sequences, i.e., 5′-UAA, 5′-UAG and 5′-UGA (the corresponding DNA sequences are 5′-TAA, 5′-TAG and 5′-TGA, respectively).

In some embodiments, “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, in some cases, the terms “stop codon region” and “translation termination codon region” refer to a portion of such an mRNA or gene that encompasses from about 25 to about 50 contiguous nucleotides in either direction (i.e., 5′ or 3′) from a translation termination codon. Consequently, the “start codon region” (or “translation initiation codon region”) and the “stop codon region” (or “translation termination codon region”) are all regions that may be targeted effectively with the antisense compounds described herein.

The open reading frame (ORF) or “coding region,” which is known in the art to refer to the region between the translation initiation codon and the translation termination codon, is also a region which may be targeted effectively. In some embodiments, a targeted region is the intragenic region encompassing the translation initiation or termination codon of the open reading frame (ORF) of a gene.

Another target region includes the 5′ untranslated region (5′-UTR), known in the art to refer to the portion of an mRNA in the 5′ direction from the translation initiation codon, and thus including nucleotides between the 5′ cap site and the translation initiation codon of an mRNA (or corresponding nucleotides on the gene). Another exemplary target region includes the 3′ untranslated region (3′-UTR), known in the art to refer to the portion of an mRNA in the 3′ direction from the translation termination codon, and thus including nucleotides between the translation termination codon and 3′ end of an mRNA (or corresponding nucleotides on the gene). The 5′ cap site of an mRNA comprises an N7-methylated guanosine residue joined to the 5-most residue of the mRNA via a 5-5′ triphosphate linkage. The 5′ cap region of an mRNA is considered to include the 5′ cap structure itself as well as the first 50 nucleotides adjacent to the cap site. Another target region is the 5′ cap region.

Although some eukaryotic mRNA transcripts are directly translated, many contain one or more regions, known as “introns,” which are excised from a transcript before it is translated. The remaining (and therefore translated) regions are known as “exons” and are spliced together to form a continuous mRNA sequence. In some embodiments, targeting splice sites, i.e., intron-exon junctions or exon-intron junctions, is useful in situations where aberrant splicing is implicated in disease, or where an overproduction of a particular splice product is implicated in disease. An aberrant fusion junction due to rearrangement or deletion is another embodiment of a target site. mRNA transcripts produced via the process of splicing of two (or more) mRNAs from different gene sources are known as “fusion transcripts”. Introns can be effectively targeted using antisense compounds targeted to, for example, DNA or pre-mRNA.

In some embodiments, the antisense oligonucleotides bind to coding and/or non-coding regions of a target polynucleotide and modulate the expression and/or function of the target molecule.

In some embodiments, the antisense oligonucleotides bind to sense polynucleotides and modulate the expression and/or function of the target molecule.

Alternative RNA transcripts can be produced from the same genomic region of DNA. These alternative transcripts are generally known as “variants”. More specifically, “pre-mRNA variants” are transcripts produced from the same genomic DNA that differ from other transcripts produced from the same genomic DNA in either their start or stop position and contain both intronic and exonic sequence.

Upon excision of one or more exon or intron regions, or portions thereof during splicing, pre-mRNA variants produce smaller “mRNA variants”. Consequently, mRNA variants are processed pre-mRNA variants and each unique pre-mRNA variant must always produce a unique mRNA variant as a result of splicing. These mRNA variants are also known as “alternative splice variants”. If no splicing of the pre-mRNA variant occurs then the pre-mRNA variant is identical to the mRNA variant.

Variants can be produced through the use of alternative signals to start or stop transcription. Pre-mRNAs and mRNAs can possess more than one start codon or stop codon. Variants that originate from a pre-mRNA or mRNA that use alternative start codons are known as “alternative start variants” of that pre-mRNA or mRNA. Those transcripts that use an alternative stop codon are known as “alternative stop variants” of that pre-mRNA or mRNA. One specific type of alternative stop variant is the “polyA variant” in which the multiple transcripts produced result from the alternative selection of one of the “polyA stop signals” by the transcription machinery, thereby producing transcripts that terminate at unique polyA sites. In some embodiments, the types of variants described herein are also embodiments of target nucleic acids.

In some embodiments, the locations on the target nucleic acid to which the antisense compounds hybridize is at least a 5-nucleotide long portion of a target region to which an active antisense compound is targeted.

In some embodiments, target segments are 5-100 nucleotides in length and comprise a stretch of at least five (5) consecutive nucleotides selected from within the target segment.

Target segments can include DNA or RNA sequences that comprise at least the 5 consecutive nucleotides from the 5′-terminus of one of the target segments (the remaining nucleotides being a consecutive stretch of the same DNA or RNA beginning immediately upstream of the 54erminus of the target segment and continuing until the DNA or RNA contains about 5 to about 100 nucleotides). In some embodiments, target segments are represented by DNA or RNA sequences that comprise at least the 5 consecutive nucleotides from the 3′-terminus of one of the target segments (the remaining nucleotides being a consecutive stretch of the same DNA or RNA beginning immediately downstream of the 3′-terminus of the target segment and continuing until the DNA or RNA contains about 5 to about 100 nucleotides).

Once one or more target regions, segments or sites have been identified, antisense compounds are chosen which are sufficiently complementary to the target, i.e., hybridize sufficiently well and with sufficient specificity, to give the desired effect.

In some embodiments, specific nucleic acids are targeted by antisense oligonucleotides. Targeting an antisense compound to a particular nucleic acid may be a multistep process. In some cases, the process comprises identification of a nucleic acid sequence whose function is to be modulated. This may be, for example, a cellular gene (or mRNA transcribed from the gene) whose expression is associated with a particular disorder or disease state.

In some embodiments, oligonucleotides or anti sense compounds include antisense oligonucleotides (e.g. RNA, DNA, mimetic, chimera, analog or homolog thereof), ribozymes, external guide sequence (EGS) oligonucleotides, siRNA compounds, single- or double-stranded RNA interference (RNAi) compounds such as siRNA compounds, saRNA, aRNA, and other oligomeric compounds which hybridize to at least a portion of the target nucleic acid and modulate its function. As such, they may be DNA, RNA, DNA-like, RNA-like, or mixtures thereof, or may be mimetics of one or more of these. These compounds may be single-stranded, double-stranded, circular or hairpin oligomeric compounds and may contain structural elements such as internal or terminal bulges, mismatches or loops. Antisense compounds may be linear, but can be joined or otherwise prepared to be circular and/or branched. Antisense compounds can include constructs such as, for example, two strands hybridized to form a wholly or partially double-stranded compound or a single strand with sufficient self-complementarity to allow for hybridization and formation of a fully or partially double-stranded compound. The two strands can be linked internally leaving free 3′ or 5′ termini or can be linked to form a continuous hairpin structure or loop. The hairpin structure may contain an overhang on either the 5′ or 3′ terminus producing an extension of single stranded character. The double stranded compounds optionally can include overhangs on the ends. Further modifications can include conjugate groups attached to one of the termini, selected nucleotide positions, sugar positions or to one of the internucleoside linkages. In some cases, the two strands can be linked via a non-nucleic acid moiety or linker group. When formed from only one strand, dsRNA can take the form of a self-complementary hairpin-type molecule that doubles back on itself to form a duplex. Thus, the dsRNAs can be fully or partially double stranded. Specific modulation of gene expression can be achieved by stable expression of dsRNA hairpins in transgenic cell lines, however, in some embodiments, the gene expression or function is up regulated. When formed from two strands, or a single strand that takes the form of a self-complementary hairpin-type molecule doubled back on itself to form a duplex, the two strands (or duplex-forming regions of a single strand) are complementary RNA strands that base pair in Watson-Crick fashion.

Once introduced to a system, the compounds may elicit the action of one or more enzymes or structural proteins to effect cleavage or other modification of the target nucleic acid or may work via occupancy-based mechanisms. In general, nucleic acids (including oligonucleotides) may be described as “DNA-like” (i.e., generally having one or more 2′-deoxy sugars and, generally, T rather than U bases) or “RNA-like” (i.e., generally having one or more 2′-hydroxyl or 2′-modified sugars and, generally U rather than T bases). Nucleic acid helices can adopt more than one type of structure, e.g., the A- and B-forms. In some cases, oligonucleotides which have B-form-like structure are “DNA-like” and those which have A-formlike structure are “RNA-like.” In some (chimeric) embodiments, an antisense compound may contain both A- and B-form regions.

In some embodiments, oligonucleotides or antisense compounds comprise at least one of: antisense RNA, antisense DNA, chimeric antisense oligonucleotides, antisense oligonucleotides comprising modified linkages, interference RNA (RNAi), short interfering RNA (siRNA); a micro, interfering RNA (miRNA); a small, temporal RNA (stRNA); or a short, hairpin RNA (shRNA); small RNA-induced gene activation (RNAa); small activating RNAs (saRNAs), or combinations thereof.

In some embodiments, a target segment may be employed in a screen for additional compounds that modulate the expression of Arachidonate 15-lipoxygenase (ALOX15) polynucleotides. In some cases, “modulators” are those compounds that decrease or increase the expression of a nucleic acid molecule encoding ALOX15 and which comprise at least a 5-nucleotide portion that is complementary to a target segment. In some cases, the screening method comprises the steps of contacting a target segment of a nucleic acid molecule encoding sense or natural antisense polynucleotides of ALOX15 with one or more candidate modulators, and selecting for one or more candidate modulators which decrease or increase the expression of a nucleic acid molecule encoding ALOX15 polynucleotides. Once it is shown that the candidate modulator or modulators are capable of modulating (e.g. either decreasing or increasing) the expression of a nucleic acid molecule encoding ALOX15 polynucleotides, the modulator may then be employed in further investigative studies of the function of ALOX15 polynucleotides, or for use as a research, diagnostic, or therapeutic agent.

The target segments may be also be combined with their respective complementary antisense compounds disclosed herein to form stabilized double-stranded (duplexed) oligonucleotides. Such double stranded oligonucleotide moieties may modulate target expression and regulate translation as well as RNA processing via an antisense mechanism. In some cases, the double-stranded moieties may be subject to chemical modifications. For example, such double-stranded moieties may inhibit the target by the classical hybridization of antisense strand of the duplex to the target, thereby triggering enzymatic degradation of the target.

In some embodiments, an antisense oligonucleotide targets Arachidonate 15-lipoxygenase (ALOX15) polynucleotides (e.g. accession number NM 001140.4), variants, alleles, isoforms, homologs, mutants, derivatives, fragments and complementary sequences thereto. In some cases, the oligonucleotide is an antisense molecule.

The target nucleic acid molecule is not limited to ALOX15 alone but extends to any of the isoforms, receptors, homologs and the like of ALOX15 molecules.

In some embodiments, the oligonucleotides are complementary to or bind to nucleic acid sequences of ALOX15 transcripts and modulate expression and/or function of ALOX15 molecules.

In some embodiments, oligonucleotides comprise sequences of at least 5 consecutive nucleotides to modulate expression and/or function of ALOX15 molecules.

In some embodiments, the polynucleotide targets comprise ALOX15, which includes family members thereof, variants of ALOX15; mutants of ALOX15, including SNPs; noncoding sequences of ALOX15; alleles of ALOX15; species variants, fragments and the like. In some cases, the oligonucleotide is an antisense molecule.

In some embodiments, the oligonucleotide targets ALOX15 polynucleotides and comprises: antisense RNA, interference RNA (RNAi), short interfering RNA (siRNA); micro interfering RNA (miRNA); a small, temporal RNA (stRNA); or a short, hairpin RNA (shRNA); small RNA-induced gene activation (RNAa); or, small activating RNA (saRNA).

In some embodiments, targeting of Arachidonate 15-lipoxygenase (ALOX15) polynucleotides, e.g. SEQ ID NO: 1 modulate the expression or function of this target. In some embodiments, expression or function is down-regulated as compared to a control.

In some embodiments, antisense compounds comprise sequences set forth as SEQ ID NOS: 3 and 5. These oligonucleotides can comprise one or more modified nucleotides, shorter or longer fragments, modified bonds and the like.

In some embodiments, SEQ ID NOS: 3 and 5 comprise one or more LNA nucleotides.

Enzymatic nucleic acid molecules (e.g., ribozymes) are nucleic acid molecules capable of catalyzing one or more of a variety of reactions, including the ability to repeatedly cleave other separate nucleic acid molecules in a nucleotide base sequence-specific manner. Such enzymatic nucleic acid molecules can be used, for example, to target virtually any RNA transcript.

Because of their sequence-specificity, trans-cleaving enzymatic nucleic acid molecules show promise as therapeutic agents for human disease. Enzymatic nucleic acid molecules can be designed to cleave specific RNA targets within the background of cellular RNA. Such a cleavage event renders the mRNA non-functional and abrogates protein expression from that RNA. In this manner, synthesis of a protein associated with a disease state can be selectively inhibited.

In general, enzymatic nucleic acids with RNA cleaving activity act by first binding to a target RNA. Such binding occurs through the target binding portion of an enzymatic nucleic acid which is held in close proximity to an enzymatic portion of the molecule that acts to cleave the target RNA. Thus, the enzymatic nucleic acid first recognizes and then binds a target RNA through complementary base pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets.

Several approaches such as in vitro selection (evolution) strategies have been used to evolve new nucleic acid catalysts capable of catalyzing a variety of reactions, such as cleavage and ligation of phosphodiester linkages and amide linkages.

The development of ribozymes that are optimal for catalytic activity may be employed with the embodiments of the present disclosure. The hammerhead ribozyme, for example, functions with a catalytic rate (kcat) of about 1 min-1 in the presence of saturating (10 mM) concentrations of Mg2+ cofactor. An artificial “RNA ligase” ribozyme has been shown to catalyze the corresponding self-modification reaction with a rate of about 100 min-1. In addition, it is known that certain modified hammerhead ribozymes that have substrate binding arms made of DNA catalyze RNA cleavage with multiple turn-over rates that approach 100 min-1. Finally, replacement of a specific residue within the catalytic core of the hammerhead with certain nucleotide analogues gives modified ribozymes that show as much as a 10-fold improvement in catalytic rate. These findings demonstrate that ribozymes can promote chemical transformations with catalytic rates that are significantly greater than those displayed in vitro by most natural self-cleaving ribozymes. It is then possible that the structures of certain self-cleaving ribozymes may be optimized to give maximal catalytic activity, or that entirely new RNA motifs can be made that display significantly faster rates for RNA phosphodiester cleavage.

Intermolecular cleavage of an RNA substrate by an RNA catalyst that fits the “hammerhead” model was first shown in 1987. The RNA catalyst was recovered and reacted with multiple RNA molecules, demonstrating that it was truly catalytic.

Catalytic RNAs designed based on the “hammerhead” motif have been used to cleave specific target sequences by making appropriate base changes in the catalytic RNA to maintain necessary base pairing with the target sequences. This has allowed use of the catalytic RNA to cleave specific target sequences and indicates that catalytic RNAs designed according to the “hammerhead” model may possibly cleave specific substrate RNAs in vivo.

In some embodiments, an oligonucleotide or antisense compound comprises an oligomer or polymer of ribonucleic acid (RNA) and or deoxyribonucleic acid (DNA), or a mimetic, chimera, analog or homolog thereof. This term may include oligonucleotides composed of naturally occurring nucleotides, sugars and covalent internucleoside (backbone) linkages as well as oligonucleotides having non-naturally occurring portions which function similarly. Such modified or substituted oligonucleotides may have properties such as, for example, enhanced cellular uptake, enhanced affinity for a target nucleic acid, and/or increased stability in the presence of nucleases.

In some embodiments, the antisense compound comprises an antisense portion from about 5 to about 80 nucleotides (i.e. from about 5 to about 80 linked nucleosides) in length. This refers to the length of the antisense strand or portion of the antisense compound. In other words, a single-stranded antisense compound of may comprise from 5 to about 80 nucleotides, and a double-stranded antisense compound (such as a dsRNA, for example) may comprise a sense and an antisense strand or portion of 5 to about 80 nucleotides in length. In some embodiments, the antisense has portions of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotides in length, or any range there within.

In some embodiments, the antisense compound has antisense portions of 10 to 50 nucleotides in length. In some cases, this embodies oligonucleotides having antisense portions of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length, or any range there within. In some embodiments, the oligonucleotides are 15 nucleotides in length.

In some embodiments, the antisense or oligonucleotide compounds have antisense portions of 12 or 13 to 30 nucleotides in length. In some cases, this embodies antisense compounds having antisense portions of 12, 13, 14, 15, 16, 17, 18, 19, 20, 21, 22, 23, 24, 25, 26, 27, 28, 29 or 30 nucleotides in length, or any range there within.

In some embodiments, the oligomeric compounds include variants in which a different base is present at one or more of the nucleotide positions in the compound. For example, if the first nucleotide is an adenosine, variants may be produced which contain thymidine, guanosine or cytidine at this position. This may be done at any of the positions of the antisense or dsRNA compounds. These compounds are then tested using the methods described herein to determine their ability to inhibit expression of a target nucleic acid.

In some embodiments, homology, sequence identity or complementarity, between the antisense compound and target is from about 40% to about 60%. In some embodiments, homology, sequence identity or complementarity, is from about 60% to about 70%. In some embodiments, homology, sequence identity or complementarity, is from about 70% to about 80%. In some embodiments, homology, sequence identity or complementarity, is from about 80% to about 90%. In some embodiments, homology, sequence identity or complementarity, is about 90%, about 92%, about 94%, about 95%, about 96%, about 97%, about 98%, about 99% or about 100%.

In an embodiment, the antisense oligonucleotides, such as for example, nucleic acid molecules set forth in SEQ ID NOS: 3 and 5 comprise one or more substitutions or modifications. In some embodiments, the nucleotides are substituted with locked nucleic acids (LNA).

In some embodiments, the oligonucleotides target one or more regions of the nucleic acid molecules sense and/or antisense of coding and/or non-coding sequences associated with ALOX15 and the sequences set forth as SEQ ID NOS: 1 and 2. In some cases, the oligonucleotides are also targeted to overlapping regions of SEQ ID NOS: 1 and 2.

In some embodiments, provided is a composition of one or more antisense compounds, particularly oligonucleotides, targeted to a first nucleic acid and one or more additional antisense compounds targeted to a second nucleic acid target. For example, the first target may be a particular antisense sequence of Arachidonate 15-lipoxygenase (ALOX15), and the second target may be a region from another nucleotide sequence. In some embodiments, compositions may contain two or more antisense compounds targeted to different regions of the same Arachidonate 15-lipoxygenase (ALOX15) nucleic acid target. Numerous examples of antisense compounds are illustrated herein and others may be selected from among suitable compounds known in the art. Two or more combined compounds may be used together or sequentially.

Nucleotide Modifications

In one aspect, an oligonucleotide described herein, e.g., an antisense and/or dsRNA agent comprises one or more modifications.

In some embodiments, certain oligonucleotides are chimeric oligonucleotides. In some embodiments, chimeric oligonucleotides or chimeras are oligonucleotides which contain two or more chemically distinct regions, each made up of at least one nucleotide. In some cases, these oligonucleotides contain at least one region of modified nucleotides that confers one or more beneficial properties (such as, for example, increased nuclease resistance, increased uptake into cells, increased binding affinity for the target) and a region that is a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of antisense modulation of gene expression. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art. In some embodiments, a chimeric oligonucleotide comprises at least one region modified to increase target binding affinity, and, optionally, a region that acts as a substrate for RNAse H. Affinity of an oligonucleotide for its target may be determined by measuring the Tm of an oligonucleotide target pair, which is the temperature at which the oligonucleotide and target dissociate; where dissociation is detected spectrophotometrically. The higher the Tm, the greater is the affinity of the oligonucleotide for the target.

Chimeric antisense compounds may be formed as composite structures of two or more oligonucleotides, modified oligonucleotides, oligonucleosides and/or oligonucleotides mimetics as described above. Such; compounds have also been referred to in the art as hybrids or gapmers.

In some embodiments, the region of the oligonucleotide which is modified comprises at least one nucleotide modified at the 2′ position of the sugar, e.g., a 2′-Oalkyl, 2, -0-alkyl-0-alkyl or 2′-fluoro-modified nucleotide. In some embodiments, RNA modifications include 2′-fluoro, 2′-amino and 2′ O-methyl modifications on the ribose of pyrimidines, abasic residues or an inverted base at the 3′ end of the RNA. Such oligonucleotides may have a higher Tm (i.e., higher target binding affinity) than 2′-deoxyoligonucleotides against a given target. The effect of such increased affinity may be to enhance RNAi oligonucleotide inhibition of gene expression. RNAse H is a cellular endonuclease that cleaves the RNA strand of RNA:DNA duplexes; activation of this enzyme therefore results in cleavage of the RNA target, and thus in some cases can enhance the efficiency of RNAi inhibition. Cleavage of the RNA target can be routinely demonstrated by gel electrophoresis.

In some embodiments, the oligonucleotide is modified to enhance nuclease resistance. Cells contain a variety of exo- and endo-nucleases which can degrade nucleic acids. Nuclease resistance may be measured by incubating oligonucleotides with cellular extracts or isolated nuclease solutions and measuring the extent of intact oligonucleotide remaining over time, e.g., by gel electrophoresis. Oligonucleotides which have been modified to enhance their nuclease resistance may survive intact for a longer time than unmodified oligonucleotides. In some embodiments, the oligonucleotide comprises at least one phosphorothioate modification. In some cases, oligonucleotide modifications which enhance target binding affinity are also, independently, able to enhance nuclease resistance. Enemas and intramuscular, intravitreal, intrathecal injections have been used for the administration of a variety of oligonucleotides with and without phosphorothioate bonds.

Non-limiting examples of oligonucleotides embodied herein include those comprising modified backbones, for example, phosphorothioates, phosphotriesters, methyl phosphonates, short chain alkyl or cycloalkyl intersugar linkages or short chain heteroatomic or heterocyclic intersugar linkages. In some embodiments, oligonucleotides comprise phosphorothioate backbones, or heteroatom backbones, e.g., CH2-NH-0-CH2, CH, ˜N(CH3)-0˜CH2 [known as a methylene(methylimino) or MM backbone], CH2-0˜N (CH3)˜CH2, CH2-N(CH3)-N(CH3)-CH2 and/or 0˜N (CH3)˜CH2-CH2 backbones, wherein the native phosphodiester backbone is represented as O—P—O—CH. In some embodiments, oligonucleotides have a morpholino backbone structure. In some embodiments, such as oligonucleotides having a peptide nucleic acid (PNA) backbone, the phosphodiester backbone of the oligonucleotide is replaced with a polyamide backbone, the nucleotides being bound directly or indirectly to theaza nitrogen atoms of the polyamide backbone. Oligonucleotides may also comprise one or more substituted sugar moieties. In some embodiments, oligonucleotides comprise one of the following at the 2′ position: OH, SH, SCH3, F, OCN, OCH3 OCH3, OCH3 O(CH2)n CH3, O(CH2)n NH2 or O(CH2)n CH3 where n is from 1 to about 10; CI to CIO lower alkyl, alkoxyalkoxy, substituted lower alkyl, alkaryl or aralkyl; CI; Br; CN; CF3; OCF3; 0˜, S-, or N-alkyl; 0-, S-, or N-alkenyl; SOCH3; SO2 CH3; ONO2; NO2; N3; NH2; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. A non-limiting exemplary modification includes 2′-methoxyethoxy [2-0-CH2 CH2 OCH3, also known as 2′-0-(2-methoxyethyl)]. Other modifications include 2′-methoxy (2′-0˜CH3), 2′-propoxy (2′-OCH2 CH2CH3) and 2′-fluoro (2′-F). Similar modifications may also be made at other positions on the oligonucleotide, e.g., the 3′ position of the sugar on the 3′ terminal nucleotide and the 5′ position of 5′ terminal nucleotide. Oligonucleotides may also have sugar mimetics such as cyclobutyls in place of the pentofuranosyl group.

In some embodiments, the oligonucleotides comprise a nucleobase (often referred to in the art simply as “base”) modifications or substitutions. In some embodiments, unmodified or natural nucleotides include adenine (A), guanine (G), thymine (T), cytosine (C) and uracil (U). In some embodiments, modified nucleotides include nucleotides found only infrequently or transiently in natural nucleic acids, e.g., hypoxanthine, 6-methyladenine, 5-Me pyrirnidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, as well as synthetic nucleotides, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-tmothvmine, 5-bromouracil, 5-hydroxymethyluracil, 8-azaguanine, 7-deazaguanine, N6 (6-aminohexyl)adenine and 2,6-diaminopurine. In some cases, a universal base, e.g., inosine, may be included. In some cases, the oligonucleotide comprises a 5-Me-C substitution, where this substitution may increase nucleic acid duplex stability, e.g., by 0.6-1.2° C. Modified nucleotides comprise other synthetic and natural nucleotides such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudo-uracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylquanine and 7-methyladenine, 8-azaguanine and 8-azaadenine, 7-deazagnanine and 7-deazaadenine and 3-deazaguanine and 3-deazaadenine.

In some embodiments, the oligonucleotide is chemically linked to one or more moieties or conjugates which enhance the activity or cellular uptake of the oligonucleotide. Such moieties include, but are not limited to, lipid moieties such as a cholesterol moiety, a cholesteryl moiety, an aliphatic chain, e.g., dodecandiol or undecyl residues, a polyamine or a polyethylene glycol chain, and Adamantane acetic acid.

In some embodiments, it is not necessary for all positions in a given oligonucleotide to be uniformly modified, e.g., in some cases, more than one of the modifications described herein may be incorporated in a single oligonucleotide or even at within a single nucleoside within an oligonucleotide.

In some embodiments, the oligonucleotide is conjugated with another moiety, e.g., an abasic nucleotide, polyether, polyamine, polyamide, peptide, carbohydrate, lipid, and/or polyhydrocarbon compounds. In some cases, these molecules are linked to one or more of any nucleotides comprising the nucleic acid molecule at several positions on the sugar, base or phosphate group.

The oligonucleotides described herein may be made through solid phase synthesis. Equipment for such synthesis is sold by several vendors including Applied Biosystems. Any other means for such synthesis may also be employed. In some cases, oligonucleotides comprising phosphorothioates and alkylated derivatives are prepared. Similar techniques and commercially available modified amidites and controlled-porc glass (CPG) products such as biotin, fluorescein, acridine or psoralen-modified amidites and/or CPG (available from Glen Research, Sterling Va.) may be used to synthesize fluorescently labeled, biotinylated or other modified oligonucleotides such as cholesterol-modified oligonucleotides.

In some embodiments, modifications such as the use of LNA monomers are used to enhance the potency, specificity and duration of action and broaden the routes of administration of oligonucleotides comprised of current chemistries such as MOE, ANA, FAN A, PS etc. This can be achieved by substituting some of the monomers in the current oligonucleotides by LNA monomers. The LNA modified oligonucleotide may have a size similar to the parent compound or may be larger or smaller. It some embodiments, LNA-modified oligonucleotides contain less than about 70%, less than about 60%, or less than about 50% LNA monomers, and that their sizes are between about 5 and 25 nucleotides, or between about 12 and 20 nucleotides.

In some embodiments, a modified oligonucleotide backbone comprises, but is not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3-5′ linkages, 2-5′ linked analogs of these, and those having inverted polarity wherein the adjacent pairs of nucleoside units are linked 3′-5′ to 5′-3′ or 2′-5′ to 5′-2. Various salts, mixed salts and free acid forms are also included.

In some embodiments, modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These comprise those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts.

In some embodiments, both the sugar and the internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone.

In some embodiments, the oligonucleotides comprise phosphorothioate backbones and/or oligonucleosides with heteroatom backbones, and in particular —CH2-NH-0-CH2-, —CH2-N(CH3)-0-CH2-known as a methylene (memylimino) or MMI backbone, —CH2-0-N(CH3)-CH2-, —CH2N(CH3)-N(CH3) CH2-and-0-N(CH3)-CH2-CH2- wherein the native phosphodiester backbone is represented as -0-P-0-CH2-. In some cases, oligonucleotides have morpholino backbone structures.

Modified oligonucleotides may comprise one or more substituted sugar moieties. In some cases, oligonucleotides comprise one of the following at the 2′ position: OH; F; 0-, S-, or N-alkyl; 0-, S-, or N-alkenyl; 0-, S- or N-alkynyl; or O alkyl-O-alkyl, wherein the alkyl, alkenyl and alkynyl may be substituted or unsubstituted C to CO alkyl or C2 to CO alkenyl and alkynyl. In some embodiments, the moiety comprises O (CH2)n OmCH3, O(CH2)n, OCH3, O(CH2)nNH2, O(CH2)nCH3, O(CH2)nONH2, or O(CH2n0N(CH2)nCH3)₂, where n and m can be from 1 to about 10. In some embodiments, oligonucleotides comprise one of the following at the 2′ position: C to CO, (lower alkyl, substituted lower alkyl, alkaryl, aralkyl, O-alkaryl or O-aralkyl), SH, SCH3, OCN, CI, Br, CN, CF3, 0CF3, SOCH3, S02CH3, ON02, N02, N3, NH2, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. In some embodiments, a modification comprises 2′-methoxyethoxy (2′-0-CH2CH20CH3, also known as 2′-0-(2-methoxyethyl) or 2′-MOE) i.e., an alkoxyalkoxy group. In some embodiments, a modification comprises 2′-dimethylaminooxyethoxy, i.e., a O(CH2)20N(CH3)2 group, also known as 2-DMAOE, and 2′-dimemylaminoethoxyethoxy (also known in die art as 2′-O-dimethylaminoethoxyethyl or 2′-DMAEOE), i.e., 2′-0-CH2-0-CH2-N(CH2)2.

Additional non-limiting exemplary modifications comprise 2-methoxy (2-0 CH3), 2′-aminopropoxy (2′-0 CH2CH2CH2NH2) and 2-fluoro (2′-F). Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar.

In some embodiments, a nucleotide is present in an oligonucleotide herein to increase the binding affinity of the oligonucleotide to its target. By way of non-limiting example, the nucleotide may comprise 5-substituted pyrimidines, 6-azapyrirnidines and N-2, N-6 and 0-6 substituted purines, comprising 2-aminopropyladenine, 5-propynyluracil and 5-propynylcytosine. 5-methylcytosine substitutions may increase nucleic acid duplex stability, e.g., by 0.6-1.2° C. In some cases, 5-methylcytosine substitutions are combined with 2′-Omethoxyethyl sugar modifications.

In certain embodiments, the modified oligonucleotide comprises a carrier system, e.g., to deliver the modified oligonucleotide into a cell of a mammal. Non-limiting examples of carrier systems suitable for use herein include nucleic acid-lipid particles, liposomes, micelles, virosomes, nucleic acid complexes, and mixtures thereof. In certain instances, the modified oligonucleotide molecule is complexed with a lipid such as a cationic lipid to form a lipoplex. In certain instances, the modified oligonucleotide molecule is complexed with a polymer such as a cationic polymer (e.g., polyethylenimine (PEI)) to form a polyplex. The modified oligonucleotide molecule may also be complexed with cyclodextrin or a polymer thereof. In some cases, the modified oligonucleotide molecule is encapsulated in a nucleic acid-lipid particle.

Nucleotides that Replace Phosphodiester Group

In some embodiments, an oligonucleotide comprises a nucleotide that replaces a phosphodiester group.

The substitution of one non-bridging oxygen of a phosphodiester with a sulfur atom creates the phosphorothioate (PS) linkage. A PS bond creates a new stereocenter in the nucleotide and when synthesized under standard achiral conditions creates diastereomeric mixtures of Rp and Sp at the phosphorous atom.

There are other functional groups that have been identified as replacements of the phosphodiester group in the oligonucleotide. Like phosphates and phosphorothioates, there are a variety of functional groups that are negatively charged such as phosphorodithioate (PS2) and thio-phosphoramidates. There are number of analogues that are uncharged such as phosphorodiamidate morpholino oligomer (PMO), peptide nucleic acid (PNA), phosphotriesters, and phosphonates. It has been postulated that the uncharged analogues are not only nuclease resistant, but may also be more membrane permeable; however, the size and hydrophilicity of uncharged oligonucleotides still preclude their passive diffusion across membranes.

Morpholino oligos (PMOs) use a hydrolytically stable, uncharged phosphordiamidate functional group.

Peptide nucleic acids (PNAs) are based upon the amide functional group. In some embodiments, an oligonucleotide comprises a PNA.

Nucleoside Analogues that Alter the Structure of Ribose

There are a variety of nucleotide mimics wherein the ribose or deoxyribose is modified to increase affinity for target and/or increase nuclease resistance. Modifications to all five positions of the ribose ring have been made; however, the modifications of the 2′ position of ribose have been the most studied. In some embodiments, an oligonucleotide comprises a nucleoside analogue that alters the structure of ribose.

1′ Position: Base Modifications

There are a few examples of base modifications that are designed to increase base pairing. The most well-known is the G-clamp which is a cytidine mimic that is designed to have increased affinity for guanosine bases due to hydrogen bonding through an aminoethyl group. C-5 propynyl pyrimidines are known to form more stable duplexes; however, they appear to be more toxic as well. In some embodiments, an oligonucleotide comprises a base modification at the 1′ position.

2′ Modifications

Modifications of the hydroxyl group at the 2′ position of ribose have been used to mimic the structure of the ribose ring while inhibiting ribonucleases that require the 2′0H group for hydrolysis of RNA. 2′-O-Methyl ribonucleic acids are naturally occurring nucleosides and have been shown to increase binding affinity to RNA itself while being resistant to ribonuclease. 2′-O-Methyl groups can be extensively substituted into RNAi triggers, and were the first nucleotide analogues used in “antagomirs.” 2′-O-Methoxyethyl(MOE) modification was designed to mimic the ribonuclease resistance of O-methyl, attenuate protein-oligonucleotide interactions and have increased affinity for RNA.

Fluorine is highly electronegative, and 2′-deoxy-2′-fluoro (2′-F) analogues of nucleosides adopt C3′-endo conformations characteristic of the sugars in RNA helices.

In some embodiments, an oligonucleotide comprises a base modification at the 2′ position.

4′- and 5′-Modifications

Alkoxy substituents at the 4′ position of 2′ deoxyribose mimic the conformation of ribose. In some embodiments, an oligonucleotide comprises a base modification at the 4′ position.

Bicyclic 2′-4′-Modifications

There are a variety of ribose derivatives that lock the carbohydrate ring into the 3′ endo conformation by the formation of bicyclic structures with a bridge between the 2′ oxygen and the 4′ position. The original bicyclic structure has a methylene bridging group and is termed locked nucleic acids (LNAs). The bicyclic structure “locks” the ribose into its preferred 3′ endo conformation and increases base pairing affinity. It has been shown the that incorporation of LNAs into a DNA duplex can increase melting points up to 8° C. per LNA. Subsequently, a variety of bicyclic nucleotides have been developed such as Bridged Nucleic Acids (BNAs), Ethyl-bridged (ENAs), constrained ethyl (cEt) nucleic acids and tricyclic structures with varying affinity for target sites. LNAs can be incorporated into antagomirs, splice blocking oligonucleotides, either strand of an RNAi duplex; however, like other 3′ endo conformers, LNAs are not substrates for RNAse H. On the other end of the stability spectrum from bicyclic nucleic acids are acyclic nucleic acids, most commonly called unlocked nucleic acids, which destabilizes hybridization to the target. In some embodiments, an oligonucleotide comprises a bicyclic modification.

Modification Patterns: Gapmer Overall Design

For RNAi duplexes, recognition by RISC requires RNA-like 3′-endo nucleotides and some patterns of RNA analogues. It was observed that a pattern of alternating 2′-O-methyl groups provides stability against nucleases, but not all permutations of alternating 2′-O-methyl are active RNAi agents. The fact that one may remove all 2′-hydroxy groups with alternating 2′-fluoro and 2′-O-methyl groups to produce duplexes that are resistant to nucleases and active in RNAi suggests the 2′-hydroxy group is not absolutely required for activity, but that some sites in the RNAi duplex are sensitive to the added steric bulk of the methyl group. In some embodiments, an oligonucleotide comprises one or more of the aforementioned patterns.

Conjugates

In some embodiments, oligonucleotides comprise a group conjugated—via covalent bonds—that prolongs circulation, provides targeting to tissues and/or facilitates intracellular delivery.

In some embodiments, the oligonucleotide is chemically linked to one or more moieties or conjugates that enhance the activity, cellular distribution or cellular uptake of the oligonucleotide. These moieties or conjugates can include conjugate groups covalently bound to functional groups such as primary or secondary hydroxyl groups. Exemplary conjugate groups include intercalators, reporter molecules, polyamines, polyamides, polyethylene glycols, polyethers, groups that enhance the pharmacodynamic properties of oligomers, and groups that enhance the pharmacokinetic properties of oligomers. Exemplary conjugate groups include cholesterols, lipids, phospholipids, biotin, phenazine, folate, phenanthridine, anthraquinone, acridine, fluoresceins, rhc, Jamines, coumarins, and dyes. Groups that enhance the pharmacodynamic properties, include groups that improve uptake, enhance resistance to degradation, and/or strengthen sequence-specific hybridization with the target nucleic acid. Groups that enhance the pharmacokinetic properties, include groups that improve uptake, distribution, metabolism or excretion of the compounds provided herein. Conjugate moieties include, but are not limited to, lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-Hphosphonate, a polyamine or a polyethylene glycol chain, or Adamantane acetic acid, a palmityl moiety, or an octadecylamine or hexylamino-carbonyl-oxycholesterol moiety. Oligonucleotides may also be conjugated to active drug substances, for example, aspirin, warfarin, phenylbutazone, ibuprofen, suprofen, fenbufen, ketoprofen, (S)-(+)-pranoprofen, carprofen, dansylsarcosine, 2,3,5-triiodobenzoic acid, flufenamic acid, folinic acid, a benzolhiadiazide, chlorothiazide, a diazepine, indomethicin, a barbiturate, a cephalosporin, a sulfa drug, an antidiabetic, an antibacterial or an antibiotic. In some embodiments, a moiety comprises, but is not limited to, lipid moieties such as a cholesterol moiety, cholic acid, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or Adamantane acetic acid, a palmityl moiety, or an octadecylamine or hexylamino-carbonyl-t oxycholesterol moiety.

In some embodiments, an oligonucleotide is conjugated to prolong circulation, e.g., conjugation to polyethylene glycol (PEG), which may prevents clearance by two mechanisms: the increase in molecular weight above threshold for renal clearance and the prevention of non-specific interactions with extracellular surfaces and serum components. PEG may be incorporated into nucleic acid delivery vehicles by attachment to components that non-covalently associate with the nucleic acids, e.g. PEGylated lipids and polymers; however, direct PEG conjugation may be employed to increase nucleic acid circulation times, decrease nonspecific interactions and alter biodistribution; although in some cases, the targeting is passive and the potency of the nucleic may be compromised as PEG MW increases.

Another class of molecules that can be conjugated in order to increases circulation times is the attachment of lipophilic groups such as cholesterol or other lipophilic moiety with >12 carbons which interact with serum components such as albumen and lipoproteins thereby increasing circulation times and passive accumulation in the liver. It should be remembered that extensive PS modification increases circulation times through associations with serum components, with roughly 10 PS groups required for serum binding.

Small Molecule Inhibitors of ALOX15

Provided are small molecule (MW<1000) inhibitors of lipoxygenase that work through a variety of mechanisms. Redox-type inhibitors reduce product formation by either inhibiting the reversible alteration between ferrous and ferric states of lipoxygenase's essential iron or by interacting with radical or peroxide intermediates. Competitive-type inhibitors interfere with lipoxygenase action by blocking fatty acid binding. The third class of synthesis inhibitors is characterized by an enzyme-directed, non-redox and non-competitive mechanism (allosteric inhibition).

Naturally occurring inhibitors of lipoxygenase include acetyl-11-keto-β-boswellic acid from the Boswellia serrata plant, baicalein from Scutellaria baicalensis and Scutellaria lateriflora plants, Apigenin (4′,5,7-trihydroxyflavone) found in many plants including parsley, celery, celeriac, and chamomile, and nordihydroguaiaretic acid (NDGA), which is a major natural product from the creosote bush.

Also provided are synthetic molecules that inhibit lipoxygenase:

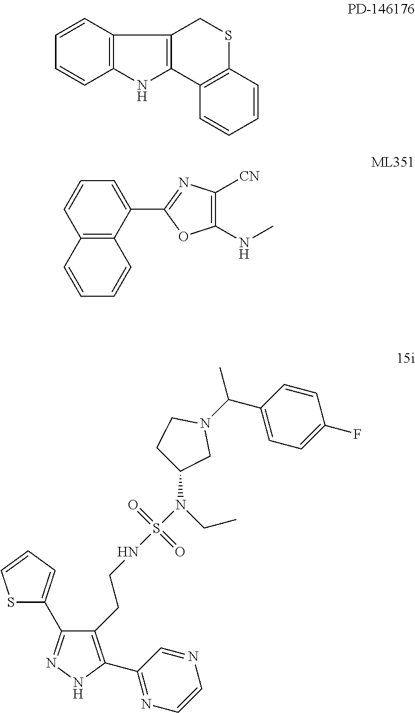

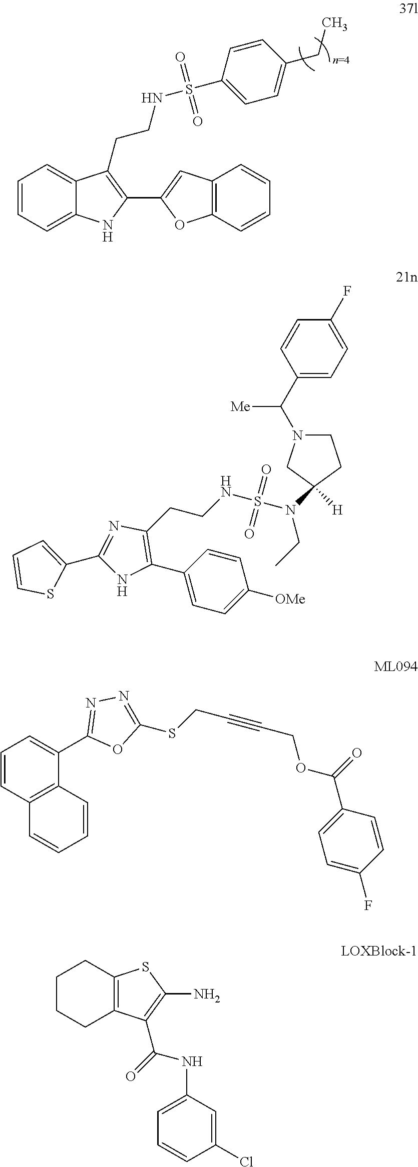

In some embodiments, the ALOX15 inhibitor is PD-146176 (CAS #4079-26-9). In some embodiments, the ALOX15 inhibitor is ML351 (CAS #847163-28-4). In some embodiments, the ALOX15 inhibitor is NDGA (CAS #500-38-9). In some embodiments, the ALOX15 inhibitor is Terameprocol (CAS #24150-24-1).

Ligands

A wide variety of entities can be coupled to the oligonucleotides or compounds disclosed herein. Exemplary moieties are ligands, which are coupled, e.g., covalently, either directly or indirectly via an intervening tether.

In some embodiments, a ligand alters the distribution, targeting or lifetime of the molecule into which it is incorporated. In some embodiments, a ligand provides an enhanced affinity for a selected target, e.g., molecule, cell or cell type, compartment, receptor e.g., a cellular or organ compartment, tissue, organ or region of the body, as, e.g., compared to a species absent such a ligand. Ligands providing enhanced affinity for a selected target are also termed targeting ligands.

Some ligands can have endosomolytic properties. The endosomolytic ligands promote the lysis of the endosome and/or transport of the composition, or its components, from the endosome to the cytoplasm of the cell. The endosomolytic ligand may be a polyanionic peptide or peptidomimetic which shows pH-dependent membrane activity and fusogenicity. In some embodiments, the endosomolytic ligand assumes its active conformation at endosomal pH. The “active” conformation is that conformation in which the endosomolytic ligand promotes lysis of the endosome and/or transport of the composition herein, or its components, from the endosome to the cytoplasm of the cell. Exemplary endosomolytic ligands include the GALA peptide, the EALA peptide, and their derivatives. In some embodiments, the endosomolytic component may contain a chemical group (e.g., an amino acid) which will undergo a change in charge or protonation in response to a change in pH. The endosomolytic component may be linear or branched. Ligands can improve transport, hybridization, and specificity properties and may also improve nuclease resistance of the resultant natural or modified oligoribonucleotide, or a polymeric molecule comprising any combination of monomers described herein and/or natural or modified ribonucleotides.

Ligands in general can include therapeutic modifiers, e.g., for enhancing uptake; diagnostic compounds or reporter groups e.g., for monitoring distribution; cross-linking agents; and nuclease-resistance conferring moieties. General examples include lipids, steroids, vitamins, sugars, proteins, peptides, polyamines, and peptide mimics.

Ligands can include a naturally occurring substance, such as a protein (e.g., human serum albumin (HSA), low-density lipoprotein (LDL), high-density lipoprotein (HDL), or globulin); a carbohydrate (e.g., a dextran, pullulan, chitin, chitosan, inulin, cyclodextrin or hyaluronic acid); or a lipid. The ligand may also be a recombinant or synthetic molecule, such as a synthetic polymer, e.g., a synthetic polyamino acid, an oligonucleotide (e.g. an aptamer). Examples of polyamino acids include polyamino acid is a polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

Ligands can also include targeting groups, e.g., a cell or tissue targeting agent, e.g., a lectin, glycoprotein, lipid or protein, e.g., an antibody, that binds to a specified cell type such as a kidney cell. A targeting group can be a thyrotropin, melanotropin, lectin, glycoprotein, surfactant protein A, Mucin carbohydrate, multivalent lactose, multivalent galactose, N-acetyl-galactosamine, N-acetyl-glucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, biotin, an RGD peptide, an RGD peptide mimetic or an aptamer.

Other examples of ligands include dyes, intercalating agents (e.g. acridines), cross-linkers (e.g. psoralene, mitomycin C), porphyrins (TPPC4, texaphyrin, Sapphyrin), polycyclic aromatic hydrocarbons (e.g., phenazine, dihydrophenazine), artificial endonucleases or a chelator (e.g. EDTA), lipophilic molecules, e.g., cholesterol, cholic acid, adamantane acetic acid, 1-pyrene butyric acid, dihydrotestosterone, 1,3-Bis-0(hexadecyl)glycerol, geranyloxyhexyl group, hexadecylglycerol, borneol, menthol, 1,3-propanediol, heptadecyl group, palmitic acid, myristic acid, 03-(oleoyl)lithocholic acid, 03-(oleoyl)cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]2, polyamino, alkyl, substituted alkyl, radiolabeled markers, enzymes, haptens (e.g. biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, or AP.

Ligands can be proteins, e.g., glycoproteins, or peptides, e.g., molecules having a specific affinity for a co-ligand, or antibodies e.g., an antibody, that binds to a specified cell type such as a cancer cell, endothelial cell, or bone cell. Ligands may also include hormones and hormone receptors. They can also include non-peptidic species, such as lipids, lectins, carbohydrates, vitamins, cofactors, multivalent lactose, multivalent galactose, N-acetyl-galactosamine, N-acetyl-glucosamine multivalent mannose, multivalent fucose, or aptamers. The ligand can be, for example, a lipopolysaccharide, an activator of p38 MAP kinase, or an activator of NF-KB.

The ligand can be a substance, e.g., a drug, which can increase the uptake of the iRNA agent into the cell, for example, by disrupting the cell's cytoskeleton, e.g., by disrupting the cell's microtubules, microfilaments, and/or intermediate filaments. The drug can be, for example, taxon, vincristine, vinblastine, cytochalasin, nocodazole, japlakinolide, latrunculin A, phalloidin, swinholide A, indanocine, or myoservin.

The ligand can increase the uptake of the oligonucleotide into the cell by activating an inflammatory response, for example. Exemplary ligands that would have such an effect include tumor necrosis factor alpha (TNF alpha), interleukin-1 beta, or gamma interferon.

In another aspect, the ligand is a moiety, e.g., a vitamin, which is taken up by a target cell, e.g., a proliferating cell. These are particularly useful for treating disorders characterized by unwanted cell proliferation, e.g., of the malignant or non-malignant type, e.g., cancer cells. Exemplary vitamins include vitamin A, E, and K. Other exemplary vitamins include B vitamins, e.g., folic acid, B12, riboflavin, biotin, pyridoxal or other vitamins or nutrients taken up by cancer cells. Also included are HAS, low density lipoprotein (LDL) and high-density lipoprotein (HDL). In another aspect, the ligand is a cell-permeation agent, e.g., a helical cell-permeation agent. In some cases, the agent is amphipathic. An exemplary agent is a peptide such as tat or antennopedia. If the agent is a peptide, it can be modified, including a peptidylmimetic, invertomers, non-peptide or pseudo-peptide linkages, and use of D-amino acids. The helical agent may be an alpha-helical agent, which may have a lipophilic and a lipophobic phase.

The ligand can be a peptide or peptidomimetic. A peptidomimetic (also referred to herein as an oligopeptidomimetic) is a molecule capable of folding into a defined three-dimensional structure similar to a natural peptide. The peptide or peptidomimetic moiety can be about 5-50 amino acids long, e.g., about 5, 10, 15, 20, 25, 30, 35, 40, 45, or 50 amino acids long. A peptide or peptidomimetic can be, for example, a cell permeation peptide, cationic peptide, amphipathic peptide, or hydrophobic peptide (e.g., consisting primarily of Tyr, Trp or Phe). The peptide moiety can be a dendrimer peptide, constrained peptide or crosslinked peptide. In another alternative, the peptide moiety can include a hydrophobic membrane translocation sequence (MTS). An exemplary hydrophobic MTS-containing peptide is RFGF having the amino acid sequence AAVALLPAVLLALLAP (SEQ ID NO: 26). An RFGF analogue (e.g., amino acid sequence AALLPVLLAAP (SEQ ID NO: 27) containing a hydrophobic MTS can also be a targeting moiety. The peptide moiety can be a “delivery” peptide, which can carry large polar molecules including peptides, oligonucleotides, and protein across cell membranes. For example, sequences from the HIV Tat protein (GRKKRRQRRRPPQ) (SEQ ID NO: 28) and the Drosophila Antennapedia protein (RQIKIWFQNRRMKWK) (SEQ ID NO: 29) have been found to be capable of functioning as delivery peptides. A peptide or peptidomimetic can be encoded by a random sequence of DNA, such as a peptide identified from a phage-display library, or one-bead-one-compound (OBOC) combinatorial library. In some cases, the peptide or peptidomimetic tethered to an iRNA agent via an incorporated monomer unit is a cell targeting peptide such as an arginine-glycine-aspartic acid (RGD)-peptide, or RGD mimic. A peptide moiety can range in length from about 5 amino acids to about 40 amino acids. The peptide moieties can have a structural modification, such as to increase stability or direct conformational properties. Any of the structural modifications described below can be utilized. An RGD peptide moiety can be used to target a tumor cell, such as an endothelial tumor cell or a breast cancer tumor cell. An RGD peptide can facilitate targeting of an iRNA agent to tumors of a variety of other tissues, including the lung, kidney, spleen, or liver. In some cases, the RGD peptide will facilitate targeting of an iRNA agent to the kidney. The RGD peptide can be linear or cyclic, and can be modified, e.g., glycosylated or methylated to facilitate targeting to specific tissues. For example, a glycosylated RGD peptide can deliver an iRNA agent to a tumor cell expressing yB3. Peptides that target markers enriched in proliferating cells can be used. E.g., RGD containing peptides and peptidomimetics can target cancer cells, in particular cells that exhibit an integrin. Thus, one could use RGD peptides, cyclic peptides containing RGD, RGD peptides that include D-amino acids, as well as synthetic RGD mimics. In addition to RGD, one can use other moieties that target the integrin ligand. Generally, such ligands can be used to control proliferating cells and angiogenesis.

In some embodiments, a “cell permeation peptide” is capable of permeating a cell, e.g., a microbial cell, such as a bacterial or fungal cell, or a mammalian cell, such as a human cell. A microbial cell-permeating peptide can be, for example, an a-helical linear peptide (e.g., LL-37 or Ceropin PI), a disulfide bond-containing peptide (e.g., a-defensin, β-defensin or bactenecin), or a peptide containing only one or two dominating amino acids (e.g., PR-39 or indolicidin). A cell permeation peptide can also include a nuclear localization signal (NLS). For example, a cell permeation peptide can be a bipartite amphipathic peptide, such as MPG, which is derived from the fusion peptide domain of HIV-1 gp41 and the NLS of SV40 large T antigen.

In some embodiments, a targeting peptide can be an amphipathic a-helical peptide. Exemplary amphipathic a-helical peptides include, but are not limited to, cecropins, lycotoxins, paradaxins, buforin, CPF, bombinin-like peptide (BLP), cathelicidins, ceratotoxins, S. clava peptides, hagfish intestinal antimicrobial peptides (HFIAPs), magainines, brevinins-2, dermaseptins, melittins, pleurocidin, H2A peptides, Xenopus peptides, esculentinis-1, and caerins. A number of factors may be considered to maintain the integrity of helix stability. For example, a maximum number of helix stabilization residues will be utilized (e.g., leu, ala, or lys), and a minimum number helix destabilization residues will be utilized (e.g., proline, or cyclic monomeric units. The capping residue will be considered (for example Gly is an exemplary N-capping residue and/or C-terminal amidation can be used to provide an extra H-bond to stabilize the helix. Formation of salt bridges between residues with opposite charges, separated by i±3, or i±4 positions can provide stability. For example, cationic residues such as lysine, arginine, homo-arginine, ornithine or histidine can form salt bridges with the anionic residues glutamate or aspartate.

Peptide and peptidomimetic ligands include those having naturally occurring or modified peptides, e.g., D or L peptides; α, β, or γ peptides; N-methyl peptides; azapeptides; peptides having one or more amide, i.e., peptide, linkages replaced with one or more urea, thiourea, carbamate, or sulfonyl urea linkages; or cyclic peptides.

The targeting ligand can be any ligand that is capable of targeting a specific receptor. Examples are: folate, GalNAc, galactose, mannose, mannose-6P, clusters of sugars such as GalNAc cluster, mannose cluster, galactose cluster, or an apatamer. A cluster is a combination of two or more sugar units. The targeting ligands also include integrin receptor ligands, Chemokine receptor ligands, transferrin, biotin, serotonin receptor ligands, PSMA, endothelin, GCPII, somatostatin, LDL and HDL ligands. The ligands can also be based on nucleic acid, e.g., an aptamer. The aptamer can be unmodified or have any combination of modifications disclosed herein.

Endosomal release agents include imidazoles, poly or oligoimidazoles, PEIs, peptides, fusogenic peptides, polycaboxylates, polyacations, masked oligo or poly cations or anions, acetals, polyacetals, ketals/polyketyals, orthoesters, polymers with masked or unmasked cationic or anionic charges, dendrimers with masked or unmasked cationic or anionic charges.

PK modulator stands for pharmacokinetic modulator. PK modulators may include lipophiles, bile acids, steroids, phospholipid analogues, peptides, protein binding agents, PEG, vitamins, etc. Exemplary PK modulator include, but are not limited to, cholesterol, fatty acids, cholic acid, lithocholic acid, dialkylglycerides, diacylglyceride, phospholipids, sphingo lipids, naproxen, ibuprofen, vitamin E, biotin etc. Oligonucleotides that comprise a number of phosphorothioate linkages are also known to bind to serum protein, thus short oligonucleotides, e.g. oligonucleotides of about 5 bases, 10 bases, 15 bases or 20 bases, comprising multiple of phosphorothioate linkages in the backbone are also amenable to the present embodiments as ligands (e.g. as PK modulating ligands).

In addition, aptamers that bind serum components (e.g. serum proteins) are also amenable as PK modulating ligands.

When two or more ligands are present, the ligands can all have same properties, all have different properties or some ligands have the same properties while others have different properties. For example, a ligand can have targeting properties, have endosomolytic activity or have PK modulating properties. In some embodiments, all the ligands have different properties.

Ligands can be coupled to the oligonucleotides at various places, for example, 3′-end, 5′-end, and/or at an internal position. In some embodiments, the ligand is attached to the oligonucleotides via an intervening tether, e.g. a carrier described herein. The ligand or tethered ligand may be present on a monomer when said monomer is incorporated into the growing strand. In some embodiments, the ligand may be incorporated via coupling to a “precursor” monomer after said “precursor” monomer has been incorporated into the growing strand. For example, a monomer having, e.g., an amino-terminated tether (i.e., having no associated ligand), e.g., TAP-(CH2)nNH2 may be incorporated into a growing oligonucleotide strand. In a subsequent operation, i.e., after incorporation of the precursor monomer into the strand, a ligand having an electrophilic group, e.g., a pentafluorophenyl ester or aldehyde group, can subsequently be attached to the precursor monomer by coupling the electrophilic group of the ligand with the terminal nucleophilic group of the precursor monomer's tether. In another example, a monomer having a chemical group suitable for taking part in Click Chemistry reaction may be incorporated e.g., an azide or alkyne terminated tether/linker. In a subsequent operation, i.e., after incorporation of the precursor monomer into the strand, a ligand having complementary chemical group, e.g. an alkyne or azide can be attached to the precursor monomer by coupling the alkyne and the azide together.

For double-stranded oligonucleotides, ligands can be attached to one or both strands. In some embodiments, a double-stranded RNA agent contains a ligand conjugated to the sense strand. In some embodiments, a double-stranded RNA agent contains a ligand conjugated to the antisense strand.

In some embodiments, a ligand can be conjugated to nucleobases, sugar moieties, or internucleosidic linkages of nucleic acid molecules. Conjugation to purine nucleobases or derivatives thereof can occur at any position including, endocyclic and exocyclic atoms. In some embodiments, the 2-, 6-, 7-, or 8-positions of a purine nucleobase are attached to a conjugate moiety. Conjugation to pyrimidine nucleobases or derivatives thereof can also occur at any position. In some embodiments, the 2-, 5-, and 6-positions of a pyrimidine nucleobase can be substituted with a conjugate moiety. Conjugation to sugar moieties of nucleosides can occur at any carbon atom. Example carbon atoms of a sugar moiety that can be attached to a conjugate moiety include the 2′, 3′, and 5′ carbon atoms. The Γ position can also be attached to a conjugate moiety, such as in an abasic residue. Internucleosidic linkages can also bear conjugate moieties. For phosphorus-containing linkages (e.g., phosphodiester, phosphorothioate, phosphorodithiotate, phosphoroamidate, and the like), the conjugate moiety can be attached directly to the phosphorus atom or to an O, N, or S atom bound to the phosphorus atom. For amine- or amide-containing internucleosidic linkages (e.g., PNA), the conjugate moiety can be attached to the nitrogen atom of the amine or amide or to an adjacent carbon atom.

Any suitable ligand in the field of RNA interference may be used, although the ligand is typically a carbohydrate e.g. monosaccharide (such as GalNAc), disaccharide, trisaccharide, tetrasaccharide, polysaccharide. Linkers that conjugate the ligand to the nucleic acid include those discussed above. For example, the ligand can be one or more GalNAc (N-acetylglucosamine) derivatives attached through a bivalent or trivalent branched linker.

Cleavable Linking Groups

A cleavable linking group is one which is sufficiently stable outside the cell, but which upon entry into a target cell is cleaved to release the two parts the linker is holding together. In some embodiments, a compound, e.g., an oligonucleotide, described herein comprises or is connected to a moiety comprising a cleavable linking group. In some embodiments, the cleavable linking group is cleaved at least 10 times or more, which may be at least 100 times faster in the target cell or under a first reference condition (which can, e.g., be selected to mimic or represent intracellular conditions) than in the blood of a subject, or under a second reference condition (which can, e.g., be selected to mimic or represent conditions found in the blood or serum).

Cleavable linking groups are susceptible to cleavage agents, e.g., pH, redox potential or the presence of degradative molecules. Generally, cleavage agents are more prevalent or found at higher levels or activities inside cells than in serum or blood.

Examples of such degradative agents include: redox agents which are selected for particular substrates or which have no substrate specificity, including, e.g., oxidative or reductive enzymes or reductive agents such as mercaptans, present in cells, that can degrade a redox cleavable linking group by reduction; esterases; endosomes or agents that can create an acidic environment, e.g., those that result in a pH of five or lower; enzymes that can hydrolyze or degrade an acid cleavable linking group by acting as a general acid, peptidases (which can be substrate specific), and phosphatases.

A cleavable linkage group, such as a disulfide bond, can be susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some linkers will have a cleavable linking group that is cleaved at a preferred pH, thereby releasing the cationic lipid from the ligand inside the cell, or into the desired compartment of the cell.

A linker can include a cleavable linking group that is cleavable by a particular enzyme. The type of cleavable linking group incorporated into a linker can depend on the cell to be targeted. For example, liver targeting ligands can be linked to the cationic lipids through a linker that includes an ester group. Liver cells are rich in esterases, and therefore the linker will be cleaved more efficiently in liver cells than in cell types that are not esterase-rich. Other cell-types rich in esterases include cells of the lung, renal cortex, and testis.

Linkers that contain peptide bonds can be used when targeting cell types rich in peptidases, such as liver cells and synoviocytes. In general, the suitability of a candidate cleavable linking group can be evaluated by testing the ability of a degradative agent (or condition) to cleave the candidate linking group. It may be desirable to also test the candidate cleavable linking group for the ability to resist cleavage in the blood or when in contact with other non-target tissue. Thus one can determine the relative susceptibility to cleavage between a first and a second condition, where the first is selected to be indicative of cleavage in a target cell and the second is selected to be indicative of cleavage in other tissues or biological fluids, e.g., blood or serum. The evaluations can be carried out in cell free systems, in cells, in cell culture, in organ or tissue culture, or in whole animals. It may be useful to make initial evaluations in cell-free or culture conditions and to confirm by further evaluations in whole animals. In some embodiments, useful candidate compounds are cleaved at least 2, 4, 10 or 100 times faster in the cell (or under in vitro conditions selected to mimic intracellular conditions) as compared to blood or serum (or under in vitro conditions selected to mimic extracellular conditions).

Redox Cleavable Linking Groups

One class of cleavable linking groups is redox cleavable linking groups that are cleaved upon reduction or oxidation. An example of reductively cleavable linking group is a disulphide linking group (—S—S—). To determine if a candidate cleavable linking group is a suitable “reductively cleavable linking group,” or for example is suitable for use with a particular iRNA moiety and particular targeting agent, one can look to methods described herein. For example, a candidate can be evaluated by incubation with dithiothreitol (DTT), or other reducing agent using reagents know in the art, which mimic the rate of cleavage which would be observed in a cell, e.g., a target cell. The candidates can also be evaluated under conditions which are selected to mimic blood or serum conditions. In some embodiments, candidate compounds are cleaved by at most 10% in the blood. In some embodiments, useful candidate compounds are degraded at least 2, 4, 10 or 100 times faster in the cell (or under in vitro conditions selected to mimic intracellular conditions) as compared to blood (or under in vitro conditions selected to mimic extracellular conditions). The rate of cleavage of candidate compounds can be determined using standard enzyme kinetics assays under conditions chosen to mimic intracellular media and compared to conditions chosen to mimic extracellular media.

Phosphate-Based Cleavable Linking Groups

Phosphate-based cleavable linking groups are cleaved by agents that degrade or hydrolyze the phosphate group. An example of an agent that cleaves phosphate groups in cells are enzymes such as phosphatases in cells. Examples of phosphate-based linking groups are -0-P(0)(ORk)-0-, -0-P(S)(ORk)-0-, -0-P(S)(SRk)-0-, —S—P(0)(ORk)-0-, -0-P(0)(ORk)-S—, —S—P(0)(ORk)-S—, -0-P(S)(ORk)-S—, —S—P(S)(ORk)-0-, -0-P(0)(Rk)-0-, -0-P(S)(Rk)-0-, —S—P(0)(Rk)-0-, —S—P(S)(Rk)-0-, —S—P(0)(Rk)-S—, -0-P(S)(Rk)-S—. Some embodiments are -0-P(0)(OH)-0-, -0-P(S)(OH)-0-, -0-P(S)(SH)-0-, —S— P(0)(OH)-0-, -0-P(0)(OH)—S—, —S—P(0)(OH)—S—, -0-P(S)(OH)—S—, —S—P(S)(OH)-0-, —O— P(0)(H)-0-, -0-P(S)(H)-0-, —S—P(O)(H)-0-, —S—P(S)(H)-0-, —S—P(0)(H)—S—, -0-P(S)(H)— S—. An exemplary embodiment is -0-P(0)(OH)-0-. These candidates can be evaluated using methods analogous to those described above.

Acid Cleavable Linking Groups

Acid cleavable linking groups are linking groups that are cleaved under acidic conditions. In some embodiments acid cleavable linking groups are cleaved in an acidic environment with a pH of about 6.5 or lower (e.g., about 6.0, 5.5, 5.0, or lower), or by agents such as enzymes that can act as a general acid. In a cell, specific low pH organelles, such as endosomes and lysosomes can provide a cleaving environment for acid cleavable linking groups. Examples of acid cleavable linking groups include but are not limited to hydrazones, esters, and esters of amino acids. Acid cleavable groups can have the general formula —C═NN—C(0)0, or —OC(O). An exemplary embodiment is when the carbon attached to the oxygen of the ester (the alkoxy group) is an aryl group, substituted alkyl group, or tertiary alkyl group such as dimethyl pentyl or t-butyl. These candidates can be evaluated using methods analogous to those described above.

Ester-Based Linking Groups

Ester-based cleavable linking groups are cleaved by enzymes such as esterases and amidases in cells. Examples of ester-based cleavable linking groups include but are not limited to esters of alkylene, alkenylene and alkynylene groups. Ester cleavable linking groups have the general formula —C(0)0-, or —OC(O)—. These candidates can be evaluated using methods analogous to those described above.

Peptide-Based Cleaving Groups

Peptide-based cleavable linking groups are cleaved by enzymes such as peptidases and proteases in cells. Peptide-based cleavable linking groups are peptide bonds formed between amino acids to yield oligopeptides (e.g., dipeptides, tripeptides etc.) and polypeptides. Peptide-based cleavable groups do not include the amide group (—C(O)NH—). The amide group can be formed between any alkylene, alkenylene or alkynelene. A peptide bond is a special type of amide bond formed between amino acids to yield peptides and proteins. The peptide based cleavage group is generally limited to the peptide bond (i.e., the amide bond) formed between amino acids yielding peptides and proteins and does not include the entire amide functional group. Peptide-based cleavable linking groups have the general formula —NHCHRAC(0)NHCHRBC(0)-, where RA and RB are the R groups of the two adjacent amino acids. These candidates can be evaluated using methods analogous to those described above.

### Therapeutic Targets and Methods of Use

In some embodiments, a composition disclosed herein (e.g., dsRNA, antisense oligonucleotide, and/or small molecule compound) targets Arachidonate 15-lipoxygenase (ALOX15), including, without limitation, sense and or antisense noncoding and/or coding sequences associated with ALOX15. In some embodiments, reference to a “composition” refers to a dsRNA agent (e.g., siRNA), antisense oligonucleotide, small molecule, and/or a lipoxygenase inhibitor (e.g., ALOX15 inhibitor).

The lipoxygenases (LOXs) are a class of structurally and functionally related non-heme iron-containing dioxygenases that catalyze the oxygenation of free and esterified polyunsaturated fatty acids to produce the corresponding hydroperoxy derivatives. LOX enzymes also use alternative substrates such as phospholipids and even in plants, triglycerides. Categorized in mammals according to the positional specificity of the oxygen insertion using arachidonic acid as a substrate, several families of LOXs are known. The major products of EFA metabolism in normal human skin and keratinocytes have been demonstrated to be 12- and 15-hydroperoxy eicosatetraenoic acids from arachidonic acid and 13-hydroxy octadecadienoic acid from linoleic acid. LOXs oxygenate arachidonic acid in different positions along the carbon chain and form the corresponding 5S-, 12S- or 15S-hydroperoxides (hydro(pero)xyeicosatetraenoic acids, H(P)ETEs). Three of these enzymes are known mainly from the blood cell types in which they are strongly expressed: the 5S-lipoxygenase of leukocytes, the 12S-lipoxygenase of platelets, and the 15S-lipoxygenase of reticulocytes, eosinophils and macrophages. While these are the most widely recognized cellular sources, selective expression is documented in other tissues. For example, both the 12S- and 15S-lipoxygenases are detected in skin. The fourth of the known human lipoxygenases, a second type of 15S-lipoxygenase, was cloned from skin and this enzyme is also expressed in prostate, lung, and cornea.

The pharmaceutical compositions described herein can be used to treat a patient suffering from a condition mediated by lipoxygenase and/or leukotriene activity. In some embodiments, the condition is mediated by 15-lipoxygenase activity. In some embodiments, the condition is an inflammatory condition. In some embodiments, the pharmaceutical composition comprises an oligonucleotide (e.g., dsRNA agent and/or antisense oligonucleotide) or small molecule compound described herein.

Conditions mediated by lipoxygenase and/or leukotriene activity include, but are not limited to asthma, rheumatoid arthritis, gout, psoriasis, allergic rhinitis, respiratory distress syndrome, chronic obstructive pulmonary disease, acne, atopic dermatitis, atherosclerosis, aortic aneurysm, sickle cell disease, acute lung injury, ischemia/reperfusion injury, chronic sinusitis, nasal polyposis, inflammatory bowel disease (including, for example, ulcerative colitis and Crohn's disease), irritable bowel syndrome, cancer, tumors, respiratory syncytial virus, sepsis, endotoxin shock and myocardial infarction. In some embodiments, the condition mediated by lipoxygenase and/or leukotriene activity is an inflammatory condition. Inflammatory conditions include, but are not limited to, appendicitis, peptic, gastric or duodenal ulcers, peritonitis, pancreatitis, acute or ischemic colitis, diverticulitis, epiglottitis, achalasia, cholangitis, cholecystitis, hepatitis, inflammatory bowel disease (including, for example, Crohn's disease and ulcerative colitis), enteritis, Whipple's disease, asthma, chronic obstructive pulmonary disease, asthma, acute lung injury, ileus (including, for example, post-operative ileus), allergy, anaphylactic shock, immune complex disease, organ ischemia, reperfusion injury, organ necrosis, hay fever, sepsis, septicemia, endotoxic shock, cachexia, hyperpyrexia, eosinophilic granuloma, granulomatosis, sarcoidosis, septic abortion, epididymitis, vaginitis, prostatitis, urethritis, bronchitis, emphysema, allergic rhinitis, chronic sinusitis, nasal polyposis, cystic fibrosis, pneumonitis, pneumoultramicroscopic silicovolcanoconiosis, alvealitis, bronchiolitis, pharyngitis, pleurisy, sinusitis, influenza, respiratory syncytial virus, herpes, disseminated bacteremia, Dengue fever, candidiasis, malaria, filariasis, amebiasis, hydatid cysts, burns, dermatitis, dermatomyositis, sunburn, urticaria, warts, wheals, vasulitis, angiitis, endocarditis, arteritis, atherosclerosis, thrombophlebitis, pericarditis, myocarditis, myocardial ischemia, periarteritis nodosa, rheumatic fever, eosinophilic esophagitis, hypereosinophilic syndromes, Alzheimer's disease, coeliac disease, congestive heart failure, adult respiratory distress syndrome, meningitis, encephalitis, multiple sclerosis, cerebral infarction, cerebral embolism, Guillame-Barre syndrome, neuritis, neuralgia, spinal cord injury, paralysis, uveitis, arthritides, arthralgias, osteomyelitis, fasciitis, Paget's disease, gout, periodontal disease, rheumatoid arthritis, synovitis, myasthenia gravis, thryoiditis, systemic lupus erythematosus, Goodpasture's syndrome, Behcet's syndrome, allograft rejection, graft-versus-host disease, Type I diabetes, ankylosing spondylitis, Berger's disease, Type II diabetes, Retier's syndrome, or Hodgkin's disease.

In some embodiments, a composition described herein (e.g., dsRNA, antisense oligonucleotide, and/or small molecule compound) is used in methods for silencing expression of a target sequence. In one aspect, provided herein are in vitro and in vivo methods for treatment of a disease or disorder in a mammal by downregulating or silencing the transcription and/or translation of a target gene of interest. In some embodiments, provided are methods for introducing a composition that silences expression (e.g., mRNA and/or protein levels) of a target sequence into a cell by contacting the cell with an oligonucleotide described herein. In some embodiments, provided are methods for in vivo delivery of an oligonucleotide that silences expression of a target sequence by administering to a mammal an oligonucleotide described herein. Administration of the oligonucleotide can be by any route known in the art, such as, e.g., oral, intranasal, intravenous, intraperitoneal, intramuscular, intra-articular, intralesional, intratracheal, subcutaneous, or intradermal. In some cases, the oligonucleotide is modified.

### Formulations, Dosing, and Administration

The compounds provided herein (e.g., dsRNA agents, antisense oligonucleotides, and/or small molecules) may be admixed, encapsulated, conjugated or otherwise associated with other molecules, molecule structures or mixtures of compounds, as for example, liposomes, receptor-targeted molecules, nasal or other formulations, for assisting in uptake, distribution and/or absorption. In some embodiments, the composition is administered in buffer.

In some embodiments, compounds described herein can be formulated for administration to a subject. A formulated composition can assume a variety of states. In some examples, the composition is at least partially crystalline, uniformly crystalline, and/or anhydrous (e.g., less than 80, 50, 30, 20, or 10% water). In another example, the compound is in an aqueous phase, e.g., in a solution that includes water.

The aqueous phase or the crystalline compositions can, e.g., be incorporated into a delivery vehicle, e.g., a liposome (particularly for the aqueous phase) or a particle (e.g., a microparticle as can be appropriate for a crystalline composition). Generally, the composition is formulated in a manner that is compatible with the intended method of administration, as described herein. For example, in particular embodiments the composition is prepared by at least one of the following methods: spray drying, lyophilization, vacuum drying, evaporation, fluid bed drying, or a combination of these techniques; or sonication with a lipid, freeze-drying, condensation and other self-assembly.

A preparation of the compound can be formulated in combination with another agent, e.g., in the case of an siRNA, the other agent may be another therapeutic agent or an agent that stabilizes a siRNA, e.g., a protein that complexes with siRNA to form an iRNP. Still other agents include chelators, e.g., EDTA (e.g., to remove divalent cations such as Mg2+), salts, RNAse inhibitors (e.g., a broad specificity RNAse inhibitor such as RNAsin) and so forth.

In some embodiments, the compound preparation includes another compound, e.g., a second siRNA that can mediate RNAi with respect to a second gene, or with respect to the same gene. Still other preparation can include at least 3, 5, ten, twenty, fifty, or a hundred or more different siRNA species. Such siRNAs can mediate RNAi with respect to a similar number of different genes.

In some embodiments, the preparation includes at least a second therapeutic agent (e.g., an agent other than a RNA or a DNA). For example, a composition for the treatment of a viral disease, e.g., HIV, might include a known antiviral agent (e.g., a protease inhibitor or reverse transcriptase inhibitor). In another example, a siRNA composition for the treatment of a cancer might further comprise a chemotherapeutic agent.

Although, the compositions do not need to be administered in the context of a vector in order to modulate a target expression and or function, some embodiments relate to expression vector constructs for the expression of oligonucleotides, comprising promoters, hybrid promoter gene sequences and possessing a strong constitutive promoter activity, or a promoter activity which can be induced in the desired case.

In some embodiments, a method of treatment involves administering at least one of the oligonucleotides described herein with a suitable nucleic acid delivery system. In some embodiments, that system includes a non-viral vector operably linked to the polynucleotide. Examples of such nonviral vectors include the oligonucleotide alone (e.g. any one or more of SEQ ID NOS: 3 and 5) or in combination with a suitable protein, polysaccharide or lipid formulation.

Additionally suitable nucleic acid delivery systems include viral vector, typically sequence from at least one of an adenovirus, adenovirus-associated virus (AAV), helper-dependent adenovirus, retrovirus, or hemagglutinatin virus of Japan-liposome (HVJ) complex. In some cases, the viral vector comprises a strong eukaryotic promoter operably linked to the polynucleotide e.g., a cytomegalovirus (CMV) promoter.

Additionally suitable vectors include viral vectors, fusion proteins and chemical conjugates. Retroviral vectors include Moloney murine leukemia viruses and HIV-based viruses. One exemplary HTV-based viral vector comprises at least two vectors wherein the gag and pol genes are from an HTV genome and the env gene is from another virus. DNA viral vectors may be used. These vectors include pox vectors such as orthopox or avipox vectors, herpesvirus vectors such as a herpes simplex I virus (HSV) vector, Adenovirus Vectors and Adeno-associated Virus Vectors.

The compositions herein encompass any pharmaceutically acceptable salts, esters, or salts of such esters, or any other compound which, upon administration to an animal, including a human, is capable of providing (directly or indirectly) the biologically active metabolite or residue thereof.

In some embodiments, pharmaceutically acceptable salts include physiologically and pharmaceutically acceptable salts of the compositions herein: i.e., salts that retain the desired biological activity of the parent compound and do not impart undesired toxicological effects thereto.

Pharmaceutical compositions and formulations described herein may comprise an antisense compound, dsRNA agent, or small molecule compound disclosed herein. The pharmaceutical compositions may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer, intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration.

The compositions, e.g., oligonucleotides, can be linked or conjugated with agents that provide desirable pharmaceutical or pharmacodynamic properties. For example, the composition can be coupled to any substance, known in the art to promote penetration or transport across the blood-brain barrier, such as an antibody to the transferrin receptor, and administered by intravenous injection. The composition can be linked with a viral vector, for example, that makes the antisense compound more effective and/or increases the transport of the composition across the blood-brain barrier. Osmotic blood brain barrier disruption can also be accomplished by, e.g., infusion of sugars including, but not limited to, meso erythritol, xylitol, D(+) galactose, D(+) lactose, D(+) xylose, dulcitol, myo-inositol, L(−) fructose, D(−) mannitol, D(+) glucose, D(+) arabinose, D(−) arabinose, cellobiose, D(+) maltose, D(+) raffinose, L(+) rhamnose, D(+) melibiose, D(−) ribose, adonitol, D(+) arabitol, L(−) arabitol, D(+) fucose, L(−) fucose, D(−) lyxose, L(+) lyxose, and L(−) lyxose, or amino acids including, but not limited to, glutamine, lysine, arginine, asparagine, aspartic acid, cysteine, glutamic acid, glycine, histidine, leucine, methionine, phenylalanine, proline, serine, threonine, tyrosine, valine, and taurine.

The compositions may be admixed, encapsulated, conjugated or otherwise associated with other molecules, molecule structures or mixtures of compounds, for example, liposomes, receptor-targeted molecules, intranasal, topical or other formulations, for assisting in uptake, distribution and/or absorption. For example, canonic lipids may be included in the formulation to facilitate oligonucleotide uptake. One such composition shown to facilitate uptake is LIPOFECTIN (available from GIBCO-BRL, Bethesda, Md.).

Oligonucleotides with at least one 2′-0-methoxyethyl modification may be useful for oral administration. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

The pharmaceutical formulations, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

The compositions may be formulated into any of many possible dosage forms. The compositions may be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

Pharmaceutical compositions include, but are not limited to, solutions, emulsions, foams and liposome-containing formulations. The pharmaceutical compositions and formulations may comprise one or more penetration enhancers, carriers, excipients or other active or inactive ingredients.

Emulsions are typically heterogeneous systems of one liquid dispersed in another in the form of droplets usually exceeding 0.1 um in diameter. Emulsions may contain additional components in addition to the dispersed phases, and the active drug that may be present as a solution in either the aqueous phase, oily phase or itself as a separate phase. Microemulsions are included as an embodiment of the present disclosure.

Formulations include liposomal formulations. In some embodiments, a “liposome” is a vesicle composed of amphiphilic lipids arranged in a spherical bilayer or bilayers. Liposomes are unilamellar or multilamellar vesicles which have a membrane formed from a lipophilic material and an aqueous interior that contains the composition to be delivered. Cationic liposomes are positively charged liposomes that are believed to interact with negatively charged DNA molecules to form a stable complex. Liposomes that are pH-sensitive or negatively-charged are believed to entrap DNA rather than complex with it. Both cationic and noncationic liposomes have been used to deliver DNA to cells.

Liposomes also include “sterically stabilized” liposomes, which includes liposomes comprising one or more specialized lipids. When incorporated into liposomes, these specialized lipids result in liposomes with enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety.

If necessary a carrier compound that assists in condensation can be added during the condensation reaction, e.g., by controlled addition. For example, the carrier compound can be a polymer other than a nucleic acid (e.g., spermine or spermidine). pH can also be adjusted to favor condensation.

Further description of methods for producing stable polynucleotide delivery vehicles, which incorporate a polynucleotide/cationic lipid complex as structural components of the delivery vehicle.

Commonly used techniques for preparing lipid aggregates of appropriate size for use as delivery vehicles include sonication and freeze-thaw plus extrusion. Microfluidization can be used when consistently small (50 to 200 nm) and relatively uniform aggregates are desired. These methods are readily adapted to packaging siRNA preparations into liposomes.

Liposomes that are pH-sensitive or negatively-charged entrap nucleic acid molecules rather than complex with them. Since both the nucleic acid molecules and the lipid are similarly charged, repulsion rather than complex formation occurs.

Nevertheless, some nucleic acid molecules are entrapped within the aqueous interior of these liposomes. pH-sensitive liposomes have been used to deliver DNA encoding the thymidine kinase gene to cell monolayers in culture. Expression of the exogenous gene was detected in the target cells.

One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

In some embodiments, cationic liposomes are used. Cationic liposomes possess the advantage of being able to fuse to the cell membrane. Non-cationic liposomes, although not able to fuse as efficiently with the plasma membrane, are taken up by macrophages in vivo and can be used to deliver siRNAs to macrophages.

Further advantages of liposomes include: liposomes obtained from natural phospholipids are biocompatible and biodegradable; liposomes can incorporate a wide range of water and lipid soluble drugs; liposomes can protect encapsulated siRNAs in their internal compartments from metabolism and degradation. Important considerations in the preparation of liposome formulations are the lipid surface charge, vesicle size and the aqueous volume of the liposomes.

A positively charged synthetic cationic lipid, N-[1-(2,3-dioleyloxy)propyl]-N,N,N-trimethylammonium chloride (DOTMA) can be used to form small liposomes that interact spontaneously with nucleic acid to form lipid-nucleic acid complexes which are capable of fusing with the negatively charged lipids of the cell membranes of tissue culture cells, resulting in delivery of siRNA.

A DOTMA analogue, 1,2-bis(oleoyloxy)-3-(trimethylammonia)propane (DOTAP) can be used in combination with a phospholipid to form DNA-complexing vesicles. Lipofectin™ Bethesda Research Laboratories, Gaithersburg, Md.) is an effective agent for the delivery of highly anionic nucleic acids into living tissue culture cells that comprise positively charged DOTMA liposomes which interact spontaneously with negatively charged polynucleotides to form complexes. When enough positively charged liposomes are used, the net charge on the resulting complexes is also positive. Positively charged complexes prepared in this way spontaneously attach to negatively charged cell surfaces, fuse with the plasma membrane, and efficiently deliver functional nucleic acids into, for example, tissue culture cells. Another commercially available cationic lipid, 1,2-bis(oleoyloxy)-3,3-(trimethylammonia)propane (“DOTAP”) (Boehringer Mannheim, Indianapolis, Ind.) differs from DOTMA in that the oleoyl moieties are linked by ester, rather than ether linkages.

Other reported cationic lipid compounds include those that have been conjugated to a variety of moieties including, for example, carboxyspermine which has been conjugated to one of two types of lipids and includes compounds such as 5-carboxyspermylglycine dioctaoleoylamide (“DOGS”) (Transfectam™, Promega, Madison, Wis.) and dipalmitoylphosphatidylethanolamine 5-carboxyspermyl-amide (“DPPES”) (see, e.g., U.S. Pat. No. 5,171,678).

Another cationic lipid conjugate includes derivatization of the lipid with cholesterol (“DC-Choi”) which has been formulated into liposomes in combination with DOPE. Lipopolylysine, made by conjugating polylysine to DOPE, has been reported to be effective for transfection in the presence of serum. For certain cell lines, these liposomes containing conjugated cationic lipids, are said to exhibit lower toxicity and provide more efficient transfection than the DOTMA-containing compositions. Other commercially available cationic lipid products include DMRIE and DMRIE-HP (Vical, La Jolla, Calif.) and Lipofectamine (DOSPA) (Life Technology, Inc., Gaithersburg, Md.).

Liposomal formulations may be particularly suited for topical administration. In some cases, liposomes present several advantages over other formulations. Such advantages include reduced side effects related to high systemic absorption of the administered drug, increased accumulation of the administered drug at the desired target, and the ability to administer siRNA, into the skin. In some implementations, liposomes are used for delivering siRNA to epidermal cells and also to enhance the penetration of siRNA into dermal tissues, e.g., into skin. For example, the liposomes can be applied topically.

Non-ionic liposomal systems have also been examined to determine their utility in the delivery of drugs to the skin, in particular systems comprising non-ionic surfactant and cholesterol. Non-ionic liposomal formulations comprising Novasome I (glyceryl dilaurate/cholesterol/polyoxyethylene-10-stearyl ether) and Novasome II (glyceryl distearate/cholesterol/polyoxyethylene-10-stearyl ether) were used to deliver a drug into the dermis of mouse skin. Such formulations with siRNA are useful for treating a dermatological disorder.

Liposomes that include siRNA can be made highly deformable. Such deformability can enable the liposomes to penetrate through pore that are smaller than the average radius of the liposome. For example, transfersomes are a type of deformable liposomes. Transferosomes can be made by adding surface edge activators, usually surfactants, to a standard liposomal composition. Transfersomes that include siRNA can be delivered, for example, subcutaneously by infection in order to deliver siRNA to keratinocytes in the skin. In order to cross intact mammalian skin, lipid vesicles must pass through a series of fine pores, each with a diameter less than 50 nm, under the influence of a suitable transdermal gradient. In addition, due to the lipid properties, these transferosomes can be self-optimizing (adaptive to the shape of pores, e.g., in the skin), self-repairing, and can frequently reach their targets without fragmenting, and often self-loading.

The pharmaceutical formulations and compositions may include surfactants.

In some embodiments, various penetration enhancers are utilized to effect the efficient delivery of nucleic acids, particularly oligonucleotides. In addition to aiding the diffusion of non-lipophilic drugs across cell membranes, penetration enhancers also enhance the permeability of lipophilic drugs. Penetration enhancers may be classified as belonging to one of five broad categories, i.e., surfactants, fatty acids, bile salts, chelating agents, and non-chelating non surfactants.

One of skill in the art will recognize that formulations are routinely designed according to their intended use, i.e. route of administration.

Non-limiting exemplary formulations for topical administration include those in which the oligonucleotides/compounds are in admixture with a topical delivery agent such as lipids, liposomes, fatty acids, fatty acid esters, steroids, chelating agents and surfactants. exemplary lipids and liposomes include neutral (e.g. dioleoyl-phosphatidyl DOPE etlianolaniine, dimyristoylphosphatidyl choline DMPC, di stearolyphosphatidyl choline) negative (e.g. dimyristoylphosphatidyl glycerol DMPG) and cationic (e.g. dioleoyltetramemylaminopropyl DOTAP and dioleoyl-phosphatidyl ethanolamine DOTMA).

For topical or other administration, oligonucleotides/compounds may be encapsulated within liposomes or may form complexes thereto, in particular to cationic liposomes. Alternatively, oligonucleotides may be complexed to lipids, in particular to cationic lipids.

Compositions and formulations for oral administration include powders or granules, microparticulates, nanoparticulates, suspensions or solutions in water or non-aqueous media, capsules, gel capsules, sachets, tablets or minitablets. Thickeners, flavoring agents, diluents, emulsifiers, dispersing aids or binders may be desirable. Exemplary surfactants include fatty acids and/or esters or salts thereof, bile acids and/or salts thereof. In some cases, combinations of penetration enhancers are utilized, for example, fatty acids salts in combination with bile acids salts. An exemplary combination is the sodium salt of lauric acid, capric acid and UDCA. Further penetration enhancers include polyoxyethylene-9-lauryl ether, polyoxyethylene-20-cetyl ether. Compositions may be delivered orally, in granular form including sprayed dried particles, or complexed to form micro or nanoparticles.

The pharmacokinetics and biodistribution of nanoparticles are dependent upon their size and charge. Upon iv administration, large (>200 nm) and/or highly positively charged (surface charge>20 mV) are primarily distributed among endothelial tissues and macrophages in the liver and spleen and have a half-life of circulation less than 2 hours. Reduction in size (<100 nm) and surface charge (˜0 mV) results increased circulation times. Local administration of positively charged polyplexes results in association with cells at site of application such as epithelial cells.

Compositions and formulations for intranasal administration may include sterile aqueous solutions that may also contain buffers, diluents and other suitable additives such as, but not limited to, penetration enhancers, carrier compounds and other pharmaceutically acceptable carriers or excipients.

In some cases, the poor solubility in water of some lipoxygenase inhibitors prevents these beneficial agents from broader use than they would otherwise enjoy if aqueous formulations could be prepared at therapeutically effective concentrations for administration, particularly, formulations for nasal or intratracheal.

Methods for modification of a poorly soluble or insoluble drug itself in an attempt to render it more suitable for administration include altering the morphology or molecular structure of the drug. Other methods include vehicle modification of a poorly soluble or insoluble drug and include the use of salt formation, co-solvent/solubilization, solid carrier systems, micellization, lipid vesicle, oil-water partitioning, and complexation. One approach for delivering a poorly soluble or insoluble agent is to formulate the drug as a solid particle suspension. Drugs that are insoluble in water can provide the significant benefit of stability when formulated as a suspension of particles in an aqueous medium to create a microparticulate or nanoparticulate suspension. Suspensions of solid particles having effective average size of from about 15 to about 1 micron are commonly referred to as nanosuspensions, and are most suitable for administration. These suspensions generally include small particles of insoluble compounds. Forming small particle compositions of lipoxygenase inhibitors can lead to increased therapeutic efficacy and increased therapeutic applications of the drug. In addition, small particle suspensions can be prepared having higher concentrations of the lipoxygenase inhibitor for later dilution prior to injection. Injectable formulations of lipoxygenase inhibitors could permit its use in treating a broad array of conditions mediated by lipoxygenase.

Once small particle suspensions having therapeutically effective concentrations of lipoxygenase inhibitors have been prepared, solid concentrates can also be prepared by known methods, such as lyophilization, spray-drying and/or supercritical fluid extraction. These solid concentrates can then be resuspended at the time of administration. Also, these solid concentrates may also be compounded to produce a single dosage form such as tablets, capsules, lozenges, suppositories, coated tablets, capsules, ampoules, suppositories, delayed release formulations, controlled release formulations, extended release formulations, pulsatile release formulations, immediate release formulations, gastroretentive formulations, effervescent tablets, fast melt tablets, oral liquid and sprinkle formulations. The solid concentrates may also be formulated in a form selected from the group consisting of a patch, a powder preparation for inhalation, a suspension, an ointment and an emulsion.

Small particle compositions of lipoxygenase inhibitors can also be formulated in therapeutically effective concentrations for delivery as an aerosol for respiratory delivery to the lungs, as a suspension for topical ophthalmic delivery or as a suspension for intranasal delivery.

Small particles of can be made using any appropriate method including, but not limited to, precipitation methods, mechanical/physical particle size reduction methods such as milling and homogenization, phospholipids coating methods, surfactant coating methods, spray-drying methods, supercritical fluid methods, and hot melt methods. The processes can be separated into four general categories. Each of the categories of processes share the steps of: (1) dissolving lipoxygenase inhibitor in a water miscible organic solvent to create a first solution; (2) mixing the first solution with a second solution that contains water, to precipitate the lipoxygenase inhibitor to create a pre-suspension; and, optionally, (3) adding energy to the pre-suspension in the form of high-shear mixing or heat to provide a stable form of the lipoxygenase inhibitor having the desired size ranges defined above.

Like other routes of administration, cyclodextrins and surfactants can be used to increase the solubility of drugs. The most common cyclodextrins are (α-, β-, and γ-cyclodextrins, consisting of 6, 7 and 8 α-1,4-linked glucose units, respectively. The number of these units determines the size of the cavity. Cyclodextrins are capable of forming inclusion complexes with hydrophobic molecules by taking up a whole molecule, or some part of it, into the cavity. The stability of the complex formed depends on how well the guest molecule fits into the cyclodextrin cavity. A composition comprising a lipoxygenase inhibitor and a cyclodextrin may include inclusion complexes of the lipoxygenase inhibitor and the cyclodextrin as well as lipoxygenase inhibitor and cyclodextrin that are not part of inclusion complexes. α-, β-, and γ-cyclodextrins, have limited aqueous solubility and show some toxicity when given by injection. For example, although β-cyclodextrins form the most stable complex with many drugs, they have the lowest water solubility of the cyclodextrins. Therefore, to overcome these shortfalls, the cyclodextrin structure has been chemically modified to generate a safer cyclodextrin derivative with increased solubility. The modifications are typically made at one or more of the 2, 3, or 6 position hydroxyl groups. As used herein, the term “cyclodextrin” is intended to encompass unmodified cyclodextrins as well as chemically modified derivatives thereof. Although (α-, β- and γ-cyclodextrins can be used for complex formation with lipoxygenase inhibitors, cyclodextrins may be the β- and γ-cyclodextrins, or they may be the β-cyclodextrins. Exemplary β-cyclodextrins include 2-hydroxypropyl-β-cyclodextrin, methyl-β-cyclodextrin and sulfobutyl derivatized β-cyclodextrin.).

Examples of surfactants include PEG-based nonionic surfactants polysorbate 20, polysorbate 80 and poloxamers. Additionally, cyclodextrins and surfactants can act as permeation enhancers to increase the bioavailability for poorly permeable drugs. Another approach to providing larger doses is using powder delivery formulations. Depending on the bulk density of the powder, quantities up to about 50 mg can be dosed intranasally. The pH of the solution may be modulated to affect the solubility and permeability of the poorly water-soluble drugs. The nasal mucosa can withstand buffers with pH range of 3-10.

To increase the residence time for nasal absorption of drugs after delivery, formulators may add viscosity-increasing and mucoadhesive agents to the formulations. To permit effective dosing of the formulation while maintaining an increased residence time, an in-situ gelling formulation may be used, example of a gel forming agents are gellan and pectin. Another strategy is the use of thixotropic rheological properties that have a low viscosity during actuation. Another method of utilizing mucoadhesive excipients in the formulation intended for nasal delivery is to produce microspheres of drug within the excipient.

For ease of exposition, the formulations, compositions and methods may be discussed largely with regard to unmodified compounds (e.g., oligonucleotides, small molecule compounds). It may be understood, however, that these formulations, compositions and methods can be practiced with other compounds, e.g., modified compounds, and such practice is within the scope of the present disclosure.

Surfactants

Surfactants find wide application in formulations such as emulsions (including microemulsions) and liposomes (see above). siRNA (or a precursor, e.g., a larger dsiRNA which can be processed into a siRNA, or a DNA which encodes a siRNA or precursor), antisense, and small molecule compositions can include a surfactant. In some embodiments, the composition is formulated as an emulsion that includes a surfactant. The most common way of classifying and ranking the properties of the many different types of surfactants, both natural and synthetic, is by the use of the hydrophile/lipophile balance (HLB). The nature of the hydrophilic group provides a useful means for categorizing the different surfactants used in formulations.

If the surfactant molecule is not ionized, it is classified as a nonionic surfactant. Nonionic surfactants find wide application in pharmaceutical products and are usable over a wide range of pH values. In general their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxy ethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfo succinates, and phosphates. In some cases, important members of the anionic surfactant class are the alkyl sulfates and the soaps. If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class.

If the surfactant molecule has the ability to carry either a positive or negative charge, the surfactant is classified as amphoteric. Amphoteric surfactants include acrylic acid derivatives, substituted alkylamides, N-alkylbetaines and phosphatides. The use of surfactants in drug products, formulations and in emulsions has been reviewed.

Micelles and other Membranous Formulations

The siRNA compound, e.g., a double-stranded siRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof)), antisense, and/or small molecule composition can be provided as a micellar formulation. In some embodiments, “micelles” are a particular type of molecular assembly in which amphipathic molecules are arranged in a spherical structure such that all the hydrophobic portions of the molecules are directed inward, leaving the hydrophilic portions in contact with the surrounding aqueous phase. The converse arrangement exists if the environment is hydrophobic.

A mixed micellar formulation suitable for delivery through transdermal membranes may be prepared by mixing an aqueous solution of the composition, an alkali metal Cs to C22 alkyl sulphate, and a micelle forming compounds. Exemplary micelle forming compounds include lecithin, hyaluronic acid, pharmaceutically acceptable salts of hyaluronic acid, glycolic acid, lactic acid, chamomile extract, cucumber extract, oleic acid, linoleic acid, linolenic acid, monoolein, monooleates, monolaurates, borage oil, evening of primrose oil, menthol, trihydroxy oxo cholanyl glycine and pharmaceutically acceptable salts thereof, glycerin, polyglycerin, lysine, polylysine, triolein, polyoxy ethylene ethers and analogues thereof, polidocanol alkyl ethers and analogues thereof, chenodeoxycholate, deoxycholate, and mixtures thereof. The micelle forming compounds may be added at the same time or after addition of the alkali metal alkyl sulphate. Mixed micelles will form with substantially any kind of mixing of the ingredients but vigorous mixing in order to provide smaller size micelles.

In one non-limiting method, a first micellar composition is prepared which contains the composition and at least the alkali metal alkyl sulphate. The first micellar composition is then mixed with at least three micelle forming compounds to form a mixed micellar composition. In another method, the micellar composition is prepared by mixing the composition, the alkali metal alkyl sulphate and at least one of the micelle forming compounds, followed by addition of the remaining micelle forming compounds, with vigorous mixing.

Phenol and/or m-cresol may be added to the mixed micellar composition to stabilize the formulation and protect against bacterial growth. Alternatively, phenol and/or m-cresol may be added with the micelle forming ingredients. An isotonic agent such as glycerin may also be added after formation of the mixed micellar composition.

For delivery of the micellar formulation as a spray, the formulation can be put into an aerosol dispenser and the dispenser is charged with a propellant. The propellant, which is under pressure, is in liquid form in the dispenser. The ratios of the ingredients are adjusted so that the aqueous and propellant phases become one, i.e., there is one phase. If there are two phases, it is necessary to shake the dispenser prior to dispensing a portion of the contents, e.g., through a metered valve. The dispensed dose of pharmaceutical agent is propelled from the metered valve in a fine spray.

Propellants may include hydrogen-containing chlorofluorocarbons, hydrogen-containing fluorocarbons, dimethyl ether and diethyl ether. In certain embodiments, HFA 134a (1,1,1,2 tetrafluoroethane) may be used.

The specific concentrations of the essential ingredients can be determined by experimentation. For absorption through the oral cavities, it may be desirable to increase, e.g., at least double or triple, the dosage for through injection or administration through the gastrointestinal tract.

Liposomes. For ease of exposition the formulations, compositions and methods in this section are discussed largely with regard to unmodified siRNA compounds. It may be understood, however, that these formulations, compositions and methods can be practiced with other siRNA compounds, e.g., modified siRNAs. An siRNA compound, e.g., a double-stranded siRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof) preparation can be formulated for delivery in a membranous molecular assembly, e.g., a liposome or a micelle. As used herein, the term “liposome” refers to a vesicle composed of amphiphilic lipids arranged in at least one bilayer, e.g., one bilayer or a plurality of bilayers. Liposomes include unilamellar and multilamellar vesicles that have a membrane formed from a lipophilic material and an aqueous interior. The aqueous portion contains the siRNA composition. The lipophilic material isolates the aqueous interior from an aqueous exterior, which typically does not include the siRNA composition, although in some examples, it may. Liposomes are useful for the transfer and delivery of active ingredients to the site of action. Because the liposomal membrane is structurally similar to biological membranes, when liposomes are applied to a tissue, the liposomal bilayer fuses with bilayer of the cellular membranes. As the merging of the liposome and cell progresses, the internal aqueous contents that include the siRNA are delivered into the cell where the siRNA can specifically bind to a target RNA and can mediate RNAi. In some cases the liposomes are also specifically targeted, e.g., to direct the siRNA to particular cell types.

A liposome containing a siRNA can be prepared by a variety of methods. In one example, the lipid component of a liposome is dissolved in a detergent so that micelles are formed with the lipid component. For example, the lipid component can be an amphipathic cationic lipid or lipid conjugate. The detergent can have a high critical micelle concentration and may be nonionic. Exemplary detergents include cholate, CHAPS, octylglucoside, deoxycholate, and lauroyl sarcosine. The siRNA preparation is then added to the micelles that include the lipid component. The cationic groups on the lipid interact with the siRNA and condense around the siRNA to form a liposome. After condensation, the detergent is removed, e.g., by dialysis, to yield a liposomal preparation of siRNA.

Particles

In some embodiments, an siRNA compound, e.g., a double-stranded siRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof), antisense oligonucleotide, and/or small molecule preparation may be incorporated into a particle, e.g., a microparticle. Microparticles can be produced by spray-drying, but may also be produced by other methods including lyophilization, evaporation, fluid bed drying, vacuum drying, or a combination of these techniques.

Pharmaceutical Compositions

The dsRNA agents, antisense oligonucleotides, and small molecules described herein may be formulated for pharmaceutical use. Pharmaceutically acceptable compositions may comprise a therapeutically-effective amount of one or more of the compounds in any of the embodiments herein, taken alone or formulated together with one or more pharmaceutically acceptable carriers (additives), excipient and/or diluents.

The pharmaceutical compositions may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally. Delivery using subcutaneous or intravenous methods can be particularly advantageous.

The phrase “therapeutically-effective amount” as used herein means that amount of a compound, material, or composition comprising a compound herein which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) lubricating agents, such as magnesium state, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; and (22) other non-toxic compatible substances employed in pharmaceutical formulations.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, from about 5 percent to about 70 percent, or from about 10 percent to about 30 percent.

In certain embodiments, a formulation comprises an excipient selected from the group consisting of cyclodextrins, celluloses, liposomes, micelle forming agents, e.g., bile acids, and polymeric carriers, e.g., polyesters and polyanhydrides; and a compound described herein. In certain embodiments, an aforementioned formulation renders orally bio available a compound described herein.

An agent preparation can be formulated in combination with another agent, e.g., another therapeutic agent or an agent that stabilizes a iRNA, e.g., a protein that complexes with iRNA to form an iRNP. Still other agents include chelators, e.g., EDTA (e.g., to remove divalent cations such as Mg2+), salts, RNAse inhibitors (e.g., a broad specificity RNAse inhibitor such as RNAsin) and so forth.

Methods of preparing these formulations or compositions include the step of bringing into association a compound described herein with the carrier and, optionally, one or more accessory ingredients. In general, the formulations are prepared by uniformly and intimately bringing into association a compound described herein with liquid carriers, or finely divided solid carriers, or both, and then, if necessary, shaping the product.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

The compounds described herein may be formulated for administration in any convenient way for use in human or veterinary medicine, by analogy with other pharmaceuticals.

The term “treatment” is intended to encompass also prophylaxis, therapy and cure. The patient receiving this treatment is any animal in need, including primates, in particular humans, and other mammals such as equines, cattle, swine and sheep; and poultry and pets in general.

Double-stranded RNAi agents are produced in a cell in vivo, e.g., from exogenous DNA templates that are delivered into the cell. For example, the DNA templates can be inserted into vectors and used as gene therapy vectors. Gene therapy vectors can be delivered to a subject by, for example, intravenous injection, local administration (U.S. Pat. No. 5,328,470), or by stereotactic injection (see, e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. The DNA templates, for example, can include two transcription units, one that produces a transcript that includes the top strand of a dsRNA agent and one that produces a transcript that includes the bottom strand of a dsRNA agent. When the templates are transcribed, the dsRNA agent is produced, and processed into siRNA agent fragments that mediate gene silencing.

Routes of Delivery

A composition that includes an iRNA can be delivered to a subject by a variety of routes. Exemplary routes include: intravenous, subcutaneous, topical, rectal, anal, vaginal, nasal, pulmonary, and ocular.

Methods/Routes for Administration

The present disclosure includes nasally administering to the mammal a therapeutically effective amount of the composition. As used herein, nasally administering or nasal administration includes administering the composition to the mucous membranes of the nasal passage or nasal cavity of the patient. As used herein, pharmaceutical compositions for nasal administration of a composition prepared by well-known methods to be administered, for example, as a nasal spray, nasal drop, suspension, gel, ointment, cream or powder. Administration of the composition may also take place using a nasal tampon or nasal sponge.

For topical administration, suitable formulations may include biocompatible oil, wax, gel, powder, polymer, or other liquid or solid carriers. Such formulations may be administered by applying directly to affected tissues, for example, a liquid formulation to treat infection of conjunctival tissue can be administered drop wise to the subject's eye, or a cream formulation can be administered to a wound site.

The compositions disclosed herein can be administered parenterally such as, for example, by intravenous, intramuscular, intrathecal or subcutaneous injection. Parenteral administration can be accomplished by incorporating the compositions described herein into a solution or suspension. Such solutions or suspensions may also include sterile diluents such as water for injection, saline solution, fixed oils, polyethylene glycols, glycerine, propylene glycol or other synthetic solvents. Parenteral formulations may also include antibacterial agents such as, for example, benzyl alcohol or methyl parabens, antioxidants such as, for example, ascorbic acid or sodium bisulfite and chelating agents such as EDTA. Buffers such as acetates, citrates or phosphates and agents for the adjustment of tonicity such as sodium chloride or dextrose may also be added. The parenteral preparation can be enclosed in ampules, disposable syringes or multiple dose vials made of glass or plastic.

Rectal administration includes administering the pharmaceutical compositions into the rectum or large intestine. This can be accomplished using suppositories or enemas. Suppository formulations can easily be made by methods known in the art. For example, suppository formulations can be prepared by heating glycerin to about 120 C, dissolving the pharmaceutical composition in the glycerin, mixing the heated glycerin after which purified water may be added, and pouring the hot mixture into a suppository mold.

Transdermal administration includes percutaneous absorption of the composition through the skin. Transdermal formulations include patches, ointments, creams, gels, salves and the like.

In addition to the usual meaning of administering the formulations described herein to any part, tissue or organ whose primary function is gas exchange with the external environment, “pulmonary” may in some instances include a tissue or cavity that is contingent to the respiratory tract, in particular, the sinuses. For pulmonary administration, an aerosol formulation containing the active agent, a manual pump spray, nebulizer or pressurized metered-dose inhaler as well as dry powder formulations are contemplated. Suitable formulations of this type can also include other agents, such as antistatic agents, to maintain the disclosed compounds as effective aerosols.

A drug delivery device for delivering aerosols comprises a suitable aerosol canister with a metering valve containing a pharmaceutical aerosol formulation as described and an actuator housing adapted to hold the canister and allow for drug delivery. The canister in the drug delivery device has a head space representing greater than about 15% of the total volume of the canister. Often, the polymer intended for pulmonary administration is dissolved, suspended or emulsified in a mixture of a solvent, surfactant and propellant. The mixture is maintained under pressure in a canister that has been sealed with a metering valve.

The pharmaceutical compositions described herein may be co-administered with one or more additional agents separately or in the same formulation. Such additional agents include, for example, anti-histamines, beta agonists (e.g., albuterol), antibiotics, anti-inflammatories (e.g. ibuprofen, prednisone (corticosteroid) or pentoxifylline), anti-fungals, (e.g. Amphotericin B, Fluconazole, Ketoconazol, and Itraconazol), steroids, decongestants, bronchodialators, and the like. The formulation may also contain preserving agents, solubilizing agents, chemical buffers, surfactants, emulsifiers, colorants, odorants and sweeteners.

The iRNA molecules described herein can be incorporated into pharmaceutical compositions suitable for administration. Such compositions typically include one or more species of iRNA and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The compositions described herein may be administered in a number of ways depending upon whether local or systemic treatment is desired and upon the area to be treated. Administration may be topical (including ophthalmic, vaginal, rectal, intranasal, transdermal), oral or parenteral. Parenteral administration includes intravenous drip, subcutaneous, intraperitoneal or intramuscular injection, or intrathecal or intraventricular administration.

The route and site of administration may be chosen to enhance targeting. For example, to target muscle cells, intramuscular injection into the muscles of interest would be a logical choice. Lung cells might be targeted by administering the iRNA in aerosol form. The vascular endothelial cells could be targeted by coating a balloon catheter with the iRNA and mechanically introducing the DNA.

Strategies for cytoplasmic delivery. There are a variety of strategies to facilitate cytoplasmic delivery of oligonucleotides including endosomal buffering (i.e. proton sponge), titratable amphiphiles, cell penetrating peptides and masked membrane lytic polymers.

The mechanism of endosomal buffering (i.e. proton sponge) to facilitate endosomolysis relies on the ability of agents such as polyamines to buffer endosomal/lysosomal compartments. The resistance to acidification is postulated to result in increased osmotic pressure that results in lysis of the lysosomal compartment. Titratable amphiphiles are polymers/peptides whose structure is pH-dependent in such a way that at acidic pH they are hydrophobic and membrane disruptive. Typically, titratable amphiphiles are polyanionic polymers or peptides composed of carboxylic acids that become neutral and membrane disruptive upon acidification. Cell penetrating peptides (CPPs) are cationic peptides, with a high propensity of guanidinium groups, which enter cells without any apparent membrane lysis. Masked lytic polymers are membrane disruptive polymers whose membrane interactivity is attenuated by reversible covalent modification. Like titratable amphiphiles, the mechanism of endosomolysis by masked polymers relies on the use of amphipathic polymers whose ability to lyse membranes is controlled such that the activity is only functional in the acidic environment of the endosome/lysosome. In the case of titratable amphiphiles, the mechanism of control is a reversible protonation of carboxylic acids. In the case of masked polymers, the control of membrane activity is the irreversible cleavage of a group that inhibits membrane interactivity of the polymer.

Liposomal Delivery Systems

Nucleic acids entrapped in lipids (lipoplexes) are a common vehicle for the delivery of nucleic acids. Cationic lipids form electrostatic complexes between nucleic acid and lipids. In addition to the cationic lipids, there are typically neutral or anionic helper lipids which are composed of unsaturated fatty acids and are postulated to assist in fusion between the lipoplex and the cellular membrane, and PEGylated lipids, which prevent aggregation during formulation and storage and non-specific interactions in vivo.

Lipids are water insoluble and nucleic acids are organic solvent insoluble. To mix these components in a controlled manner such that formulations are repeatable and relatively homogenous in size, detergents or water-miscible organic solvents such as ethanol are used. After formation of electrostatically-associated complexes, the amphipathic detergent or solvent is then removed by dialysis or solvent exchange. Depending on the components and the mixing procedure is possible to formulate lipoplexes that are well less than 100 nm.

Although the transfection efficiencies of lipoplexes are difficult to predict and optimization is empirical, there are a few design features that have been identified to aid transfection efficiency in vivo: pH-sensitive cationic lipids, the use of unsaturation in the lipid chains and the hydrophobic-hydrophilic balance of PEG-lipids to balance circulation times and transfection efficiencies.

There have been several studies that have shown a correlation between the pKa of the amine groups of the cationic lipid, which is buffer in the range of the endosomal/lysosomal pathway (pH 4-7), and transfection ability. To synthesize lipids with such pKa values, lipids commonly have closely-spaced amines or imidazole groups. The effect of these weakly basic amine groups in the lipoplexes produces several attractive attributes that facilitate in vivo transfection: reduced surface charge at neutral pH thereby decreasing nonspecific interactions in vivo, increased surface charge in acid environment of endosomes and lysosomes thereby increasing electrostatic interactions with the cellular membrane in these compartments and providing buffering groups that can provide endosomolytic activity via the proton sponge mechanism.

Another common motif observed in cationic and helper lipids used in lipoplexes is the presence of unsaturation in their component fatty acids with oleic (18 carbon chain with one double bond) and linoleic (18 carbons with 2 double bonds) being very common. The incorporation of these groups increases fluidity of membranes, aids in the formation of fusogenic lipid structures and facilitates the release of cationic lipids from nucleic acids.

PEG-conjugated lipids are incorporated into lipoplexes to aid in the formation of nonaggregating small complexes and for the prevention of nonspecific interactions in vivo. Due to the hydrophilicity of PEG, their lipid conjugates are not permanently associated with lipoplexes and diffuse from the complexes with dilution and interaction with amphiphilic components in vivo. This loss of PEG shielding from the surface of the lipoplexes aids in transfection efficiency. In general, longer saturated fatty acid chains increase circulation while unsaturation and shorter chains decrease circulation.

A commonly invoked tumor targeting mechanism is the Enhanced Permeability and Retention (EPR) effect, which is when nanoparticles accumulate in tumor tissue much more than they do in normal tissues due to the leaky disorganized vasculature associated with tumor tissues and their lack of lymphatic drainage. EPR-based targeting requires long circulating particles.

Polymer based delivery vehicles: Like lipoplexes, polymer-based transfection vehicles (polyplexes) provide nuclease protection and condensation of larger nucleic acids. Polyplexes are based upon cationic polymers that form electrostatic complexes with anionic nucleic acids. Polycations may be purely synthetic (such as polyethyleneimine), naturally occurring (such as histones, protamine, spermine and spermidine) or synthetic polymers based upon cationic amino acids such as ornithine, lysine and arginine.

Polycations form electrostatic complexes with polyanionic nucleic acids. The strength of the association is dependent upon the size of the nucleic acid and the size and charge density of the polycation.

There are three common strategies to improve the stability and surface charge of polyplexes to improve the circulation and targeting of ability of polyplexes: crosslinking of polycation, addition of a synthetic polyanion and conjugation of PEG.

Crosslinking, also called lateral stabilization and caging, is the formation of covalent polyamine-polyamine bonds after complexation/condensation of the nucleic acid. The crosslinking is accomplished by the addition of bifunctional, amine-reactive reagents that form a 3-D network of bonds around the nucleic acid, thereby making the polyplex resistant to displacement by salts and polyelectrolytes. The stability of the polyplexes is such that the nucleic acid is no longer active unless a mechanism of reversibility is introduced to allow for release of the nucleic acid. A common way to introduce reversibility is the use of disulfide-containing crosslinking reagent that can be reduced in the cytoplasm allowing release of nucleic acid therapeutic.

A common method to reduce the surface charge of a polyplex is the conjugation of PEG, a method commonly known as steric stabilization. The resulting PEG modified polyplexes have prolonged circulation in vivo. PEG modifications can be added to the size chains of polyamines—either before or after polyplex formation- or at the end of the polymer as a block copolymer of PEG and polycation.

Crosslinking and PEGylation are often combined to make stabilized polyplexes of reduced surface charge for systemic administration that can either be passively or actively targeted. As observed for lipoplexes, a variety of small molecule (such as GalNAc, RGD and folate) and biologic targeting ligands (such as transferrin and antibodies) have been conjugated to PEG-modified polyplexes for tissues selective targeting.

The most commonly used polymer for polyplexes- and the originator of the proton sponge mechanism-is polyethylenimine (PEI). PEI's high density of amine groups endows it with high charge density and a continuum of amine pKa's that buffer in the entire pH range of the endosome. The buffering capacity of PEI has been mimicked by the addition of weakly basic imidazole groups.

Oligonucleotide vehicle formulation. The solution conditions in which the oligonucleotide is dissolved, or its delivery vehicle is dispersed may play a role in its delivery. Hypotonic and hypertonic solution conditions may aid in cytoplasmic delivery for systemic and locally administration.

Nasal Delivery Formulations

Nasal drug delivery has been accomplished using several methods. One of the oldest methods of delivering liquids to the nasal cavity is the use of drops. Drops are advantageous as they are low-cost and relatively straightforward to manufacture. A common method for the nasal administration of liquids on the market today is the use of meter-dosed pump sprays. Meter-dosed pump sprays accurately deliver volumes between 25 and 200 μL. The particle size of the drops from pump sprays is a product of the device, patient handling, as well as the formulation, which varies based on the viscosity and surface tension of the product. Nasal formulations can also be delivered as powders. Powder drug delivery provides a high mass of active ingredients for a given volume.

Nasal Delivery Devices

For most purposes, a broad distribution of the drug on the mucosal surfaces appears desirable for drugs intended for local action or systemic absorption and for vaccines. In chronic sinusitis and nasal polyposis, targeted delivery to the middle and superior meatuses where the sinus openings are, and where the polyps originate, appears desirable.

Nasal drug delivery devices: Liquid nasal formulations are often aqueous solutions, but suspensions and emulsions can also be delivered. Liquid formulations are considered convenient particularly for topical indications where humidification counteracts the dryness and crusting often accompanying chronic nasal diseases. In traditional spray pump systems, preservatives are typically required to maintain microbiological stability in liquid formulations.

Drops delivered with pipette: Drops may be administered by sucking liquid into a glass dropper, inserting the dropper into the nostril with an extended neck before squeezing the rubber top to emit the drops.

Delivery of liquid with rhinyle catheter and squirt tube: A simple way for a physician or trained assistant to deposit drug in the nose is to insert the tip of a fine catheter or micropipette to the desired area under visual control and squirt the liquid into the desired location.

Squeeze bottles are mainly used to deliver some over-the counter (OTC) products like topical decongestants. By squeezing a partly air-filled plastic bottle, the drug is atomized when delivered from a jet outlet. The dose and particle size vary with the force applied.

Metered-dose spray pumps: The pumps typically deliver 100 μl (25-200 μl) per spray, and they offer high reproducibility of the emitted dose and plume geometry in in vitro tests. The particle size and plume geometry can vary within certain limits and depend on the properties of the pump, the formulation, the orifice of the actuator, and the force applied. Traditional spray pumps replace the emitted liquid with air, and preservatives are therefore required to prevent contamination. Pump manufacturers have developed different spray systems that avoid the need for preservatives. These systems use a collapsible bag, a movable piston, or a compressed gas to compensate for the emitted liquid volume. The solutions with a collapsible bag and a movable piston compensating for the emitted liquid volume offer the additional advantage that they can be emitted upside down, without the risk of sucking air into the dip tube and compromising the subsequent spray. This may be useful for some products where the patients are bedridden and where a head-down application is recommended. Some systems have a ball valve at the tip to prevent contamination of the liquid inside the applicator tip. Pumps have been designed with side-actuation. The pump was designed with a shorter tip to avoid contact with the sensitive mucosal surfaces. New designs to reduce the need for priming and re-priming, and pumps incorporating pressure point features to improve the dose reproducibility and dose counters and lock-out mechanisms for enhanced dose control and safety are available. Importantly, the in vivo deposition and clinical performance of metered-dose spray pumps can be enhanced for some applications by adapting the pumps to a novel breath-powered “Bi-Directional” delivery technology.

Single- and duo-dose spray devices: Metered-dose spray pumps require priming and some degree of overfill to maintain dose conformity for the labeled number of doses. For expensive drugs and vaccines intended for single administration or sporadic use and where tight control of the dose and formulation is of particular importance, single-dose or duo-dose spray devices may be employed. A simple variant of a single-dose spray device (MAD) is a nosepiece with a spray tip is fitted to a standard syringe. The liquid drug to be delivered is first drawn into the syringe and then the spray tip is fitted onto the syringe.

Nasal pressurized metered-dose inhalers (pMDIs): Many drugs intended for local nasal action are delivered by spray pumps, but some have also been delivered as nasal aerosols produced by pMDIs. The particles from a pMDI are released at a high speed and the expansion of a compressed gas. The particles emitted from the traditional pMDIs had a particle velocity much higher than a spray pump (5,200 vs. 1,500 cm/s at a distance 1-2 cm from the actuator tip). The issues related to the high particle speed and “cold Freon effect” have been reduced with hydrofluoroalkane (HFA)-based pMDI for nasal use offering lower particle speeds. Like spray pumps, nasal pMDIs produce a localized deposition on the anterior non-ciliated epithelium of the nasal vestibule and in the anterior parts of the narrow nasal valve, but due to quick evaporation of the spray delivered with a pMDI, noticeable “drip-out” may be less of an issue.

Powered nebulizers and atomizers: Nebulizers use compressed gasses (air, oxygen, and nitrogen) or ultrasonic or mechanical power to break up medical solutions and suspensions into small aerosol droplets that can be directly inhaled into the mouth or nose. The smaller particles and slow speed of the nebulized aerosol are advocated to increase penetration to the target sites in the middle and superior meatuses and the paranasal sinuses.

Powder devices: Powder medication formulations can offer advantages, including greater stability than liquid formulations and potential that preservatives may not be required. Powders tend to stick to the moist surface of the nasal mucosa before being dissolved and cleared. The use of bioadhesive excipients or agents that slow ciliary action may decrease clearance rates and improve absorption. A number of factors like moisture sensitivity, solubility, particle size, particle shape, and flow characteristics will impact deposition and absorption.

The function of nasal powder devices is usually based on one of three principles: 1. Powder sprayers with a compressible compartment to provide a pressure that when released creates a plume of powder particles fairly similar to that of a liquid spray; 2. Breath-actuated inhalers where the subject uses his own breath to inhale the powder into the nostril from a blister or capsule; and 3. Nasal insufflators describe devices consisting of a mouthpiece and a nosepiece that are fluidly connected. Delivery occurs when the subject exhales into the mouthpiece to close the velum, and the airflow carries the powder particles into the nose through the device nosepiece similar to the rhinyle catheter described above. The principle can be applied to different dispersion technologies.

It some embodiments, the present compositions and methods seek to overcome at least some of the above problems of drug solubility, deposition and delivery using the technologies and strategies that have been employed to deliver active pharmaceutical ingredients to the nasal cavity.

In some embodiments, an oligonucleotide conjugated with cholesterol or lipid containing >18 carbon atoms administered to nasal epithelium increases residence time with tissue and increases intracellular delivery resulting in mRNA reduction.

In some embodiments, an oligonucleotide is administered to nasal epithelium in a solution that is hypotonic.

In some embodiments, a small molecule inhibitor of lipoxygenase 15 is formulated with a cyclodextrin and administered to nasal epithelial tissue.

In some embodiments, a small molecule inhibitor of lipoxygenase 15 is formulated with a surfactant and administered to nasal epithelial tissue.

In some embodiments, an agent for decreasing lipoxygenase expression or activity is administered using an aerosol spray device.

In some embodiments, an agent for decreasing lipoxygenase expression or activity is formulated as powder and administered using a using a powder delivery device.

Dosing

Dosing is dependent on severity and responsiveness of the disease state to be treated, with the course of treatment lasting from several days to several months, or until a cure is effected or a diminution of the disease state is achieved. Dosing schedules can be calculated from measurements of drug accumulation in the body of the patient. Optimum dosages may vary depending on the relative potency of individual compositions, and can generally be estimated based on EC50s found to be effective in vitro and in vivo animal models. In general, dosage is from 0.001 ug to 100 g per kg of body weight, and may be given once or more daily, weekly, monthly or yearly, or even once every 2 to 20 years. Persons of ordinary skill in the art may estimate repetition rates for dosing based on measured residence times and concentrations of the drug in bodily fluids or tissues. Following successful treatment, it may be desirable to have the patient undergo maintenance therapy to prevent the recurrence of the disease state, wherein the oligonucleotide is administered in maintenance doses, ranging from 0.001 μg to 100 g per kg of body weight, once or more daily, to once every 20 years.

In one aspect, provided is a method of administering a dsRNA agent, e.g., a siRNA agent, to a subject (e.g., a human subject). The method includes administering a unit dose of the dsRNA agent, e.g., a siRNA agent, e.g., double stranded siRNA agent that (a) the double-stranded part is 14-30 nucleotides (nt) long, for example, 21-23 nt, (b) is complementary to a target RNA (e.g., an endogenous or pathogen target RNA), and, optionally, (c) includes at least one 3′ overhang 1-5 nucleotide long. In some embodiments, the unit dose is less than 10 mg per kg of bodyweight, or less than 10, 5, 2, 1, 0.5, 0.1, 0.05, 0.01, 0.005, 0.001, 0.0005, 0.0001, 0.00005 or 0.00001 mg per kg of bodyweight, and less than 200 nmole of RNA agent (e.g., about 4.4×1016 copies) per kg of bodyweight, or less than 1500, 750, 300, 150, 75, 15, 7.5, 1.5, 0.75, 0.15, 0.075, 0.015, 0.0075, 0.0015, 0.00075, 0.00015 nmole of RNA agent per kg of bodyweight.

The defined amount can be an amount effective to treat or prevent a disease or disorder, e.g., a disease or disorder associated with the target RNA. The unit dose, for example, can be administered by injection (e.g., intravenous, subcutaneous or intramuscular), an inhaled dose, or a topical application. In some embodiments dosages may be less than 10, 5, 2, 1, or 0.1 mg/kg of body weight.

In some embodiments, the unit dose is administered less frequently than once a day, e.g., less than every 2, 4, 8 or 30 days. In some embodiments, the unit dose is not administered with a frequency (e.g., not a regular frequency). For example, the unit dose may be administered a single time.

In some embodiments, the effective dose is administered with other traditional therapeutic modalities. In some embodiments, the subject has a viral infection and the modality is an antiviral agent other than a dsRNA agent, e.g., other than a siRNA agent. In some embodiments, the subject has atherosclerosis and the effective dose of a dsRNA agent, e.g., a siRNA agent, is administered in combination with, e.g., after surgical intervention, e.g., angioplasty.

In some embodiments, a subject is administered an initial dose and one or more maintenance doses of a dsRNA agent, e.g., a siRNA agent, (e.g., a precursor, e.g., a larger dsRNA agent which can be processed into a siRNA agent, or a DNA which encodes a dsRNA agent, e.g., a siRNA agent, or precursor thereof). The maintenance dose or doses can be the same or lower than the initial dose, e.g., one-half less of the initial dose. A maintenance regimen can include treating the subject with a dose or doses ranging from 0.01 μg to 15 mg/kg of body weight per day, e.g., 10, 1, 0.1, 0.01, 0.001, or 0.00001 mg per kg of bodyweight per day. The maintenance doses are, for example, administered no more than once every 2, 5, 10, or 30 days. Further, the treatment regimen may last for a period of time which will vary depending upon the nature of the particular disease, its severity and the overall condition of the patient. In certain embodiments the dosage may be delivered no more than once per day, e.g., no more than once per 24, 36, 48, or more hours, e.g., no more than once for every 5 or 8 days.

Following treatment, the patient can be monitored for changes in his condition and for alleviation of the symptoms of the disease state. The dosage of the compound may either be increased in the event the patient does not respond significantly to current dosage levels, or the dose may be decreased if an alleviation of the symptoms of the disease state is observed, if the disease state has been ablated, or if undesired side-effects are observed. The effective dose can be administered in a single dose or in two or more doses, as desired or considered appropriate under the specific circumstances. If desired to facilitate repeated or frequent infusions, implantation of a delivery device, e.g., a pump, semi-permanent stent (e.g., intravenous, intraperitoneal, intracisternal or intracapsular), or reservoir may be advisable.

In some embodiments, the composition includes a plurality of dsRNA agent species. In some embodiments, the dsRNA agent species has sequences that are non-overlapping and non-adjacent to another species with respect to a naturally occurring target sequence. In some embodiments, the plurality of dsRNA agent species is specific for different naturally occurring target genes. In some embodiments, the dsRNA agent is allele specific.

The dsRNA agents described herein can be administered to mammals, particularly large mammals such as nonhuman primates or humans in a number of ways.

In some embodiments, the administration of the dsRNA agent, e.g., a siRNA agent, composition is parenteral, e.g., intravenous (e.g., as a bolus or as a diffusible infusion), intradermal, intraperitoneal, intramuscular, intrathecal, intraventricular, intracranial, subcutaneous, transmucosal, buccal, sublingual, endoscopic, rectal, oral, vaginal, topical, pulmonary, intranasal, urethral or ocular. Administration can be provided by the subject or by another person, e.g., a health care provider. The medication can be provided in measured doses or in a dispenser which delivers a metered dose. Selected modes of delivery are discussed in more detail below.

Further provided are methods, compositions, and kits, for rectal administration or delivery of dsRNA agents described herein.

Delivery Vehicles

Naked oligonucleotides are defined as systems that contain no agents that are associated with the nucleic acid either covalently or non-covalently. The absence of any delivery vehicle requires that the oligonucleotide itself be sufficiently nuclease resistant, sufficiently long circulating and cell targeted. For small, solid-phase synthesized oligonucleotides such as those used in antisense oligonucleotides, RNAi, and innate immune stimulators, the use of nucleotide mimics provide the required drug-like properties.

Delivery Vehicles based upon complexation of nucleic acid. Complexation of oligonucleotide therapeutics with cationic agents inhibits nuclease from degrading the oligonucleotide by forming a steric barrier and by inhibiting nuclease binding by neutralizing anionic charge. The process of forming compact particles of nucleic acids from their extended chains is called condensation, which may be achieved by the addition of multiply-charged cationic species. Multiple positive charges can either be covalently attached to one another in a polycation or non-covalently associated with one another in a complex such as the surface of a cationic liposome. The resulting polycation-polyanion interaction is a colloidal dispersion where the nucleic acid particles vary in size and shape depending on the nucleic acid and the condensing cation. In general, the particles are greater than 20 nm in size, and—in the absence of agents to modulate surface charge such as polyethylene glycol (PEG)—have surface charges>20 mV.

### Additional Methods

Some embodiments relate to methods for inhibiting the expression of a target gene. The method comprises the step of administering the dsRNA agents in any of the preceding embodiments, in an amount sufficient to inhibit expression of the target gene.

Another aspect relates to a method of modulating the expression of a target gene in a cell, comprising providing to said cell a dsRNA agent described herein. In some embodiments, the target gene is ALOX15.

Drug discovery: The compounds described herein can also be applied in the areas of drug discovery and target validation. In some embodiments, the compounds and target segments identified herein are useful in drug discovery efforts to elucidate relationships that exist between Arachidonate 15-lipoxygenase (ALOX15) polynucleotides and a disease state, phenotype, or condition. These methods include detecting or modulating ALOX15 polynucleotides comprising contacting a sample, tissue, cell, or organism with the compounds described herein, measuring the nucleic acid or protein level of ALOX15 polynucleotides and/or a related phenotypic or chemical endpoint at some time after treatment, and optionally comparing the measured value to a non-treated sample or sample treated with a further compound described herein. These methods can also be performed in parallel or in combination with other experiments to determine the function of unknown genes for the process of target validation or to determine the validity of a particular gene product as a target for treatment or prevention of a particular disease, condition, or phenotype.

Assessing Up-Regulation or Inhibition of Gene Expression

Transfer of an exogenous nucleic acid into a host cell or organism can be assessed by directly detecting the presence of the nucleic acid in the cell or organism. Such detection can be achieved by several methods well known in the art. For example, the presence of the exogenous nucleic acid can be detected by Southern blot or by a polymerase chain reaction (PCR) technique using primers that specifically amplify nucleotide sequences associated with the nucleic acid. Expression of the exogenous nucleic acids can also be measured using conventional methods including gene expression analysis. For instance, mRNA produced from an exogenous nucleic acid can be detected and quantified using a Northern blot and reverse transcription PCR (RT-PCR).

Expression of RNA from the exogenous nucleic acid can also be detected by measuring an enzymatic activity or a reporter protein activity. For example, antisense modulatory activity can be measured indirectly as a decrease or increase in target nucleic acid expression as an indication that the exogenous nucleic acid is producing the effector RNA. Based on sequence conservation, primers can be designed and used to amplify coding regions of the target genes. Initially, the most highly expressed coding region from each gene can be used to build a model control gene, although any coding or non-coding region can be used. Each control gene is assembled by inserting each coding region between a reporter coding region and its poly(A) signal. These plasmids would produce an mRNA with a reporter gene in the upstream portion of the gene and a potential RNAi target in the 3′ non-coding region. The effectiveness of individual antisense oligonucleotides would be assayed by modulation of the reporter gene. Reporter genes useful in the methods herein include acetohydroxyacid synthase (AHAS), alkaline phosphatase (AP), beta galactosidase (LacZ), beta glucoronidase (GUS), chloramphenicol acetyltransferase (CAT), green fluorescent protein (GFP), red fluorescent protein (RFP), yellow fluorescent protein (YFP), cyan fluorescent protein (CFP), horseradish peroxidase (HRP), luciferase (Lac), nopaline synthase (NOS), octopine synthase (OCS), and derivatives thereof. Multiple selectable markers are available that confer resistance to ampicillin, bleomycin, chloramphenicol, gentamycin, hygromycin, kanamycin, lincomycin, methotrexate, phosphinothricin, puromycin, and tetracycline. Methods to determine modulation of a reporter gene are well known in the art, and include, but are not limited to, fluorometric methods (e.g. fluorescence spectroscopy, Fluorescence Activated Cell Sorting (FACS), fluorescence microscopy), antibiotic resistance determination.

ALOX15 protein and mRNA expression can be assayed using methods known to those of skill in the art and described elsewhere herein. For example, immunoassays such as the ELISA can be used to measure protein levels. ALOX15 ELISA assay kits are available commercially.

In embodiments, ALOX15 expression (e.g., mRNA or protein) in a sample (e.g., cells or tissues in vivo or in vitro) treated using an antisense oligonucleotide described herein is evaluated by comparison with ALOX15 expression in a control sample. For example, expression of the protein or nucleic acid can be compared using methods known to those of skill in the art with that in a mock-treated or untreated sample. Alternatively, comparison with a sample treated with a control antisense oligonucleotide (e.g., one having an altered or different sequence) can be made depending on the information desired. In some embodiments, a difference in the expression of the ALOX15 protein or nucleic acid in a treated vs. an untreated sample can be compared with the difference in expression of a different nucleic acid (including any standard deemed appropriate by the researcher, e.g., a housekeeping gene) in a treated sample vs. an untreated sample.

Observed differences can be expressed as desired, e.g., in the form of a ratio or fraction, for use in a comparison with control. In embodiments, the level of ALOX15 mRNA or protein, in a sample treated with an antisense oligonucleotide described herein, is increased or decreased by about 1.25-fold to about 10-fold or more relative to an untreated sample or a sample treated with a control nucleic acid. In embodiments, the level of ALOX15 mRNA or protein is increased or decreased by at least about 1.25-fold, at least about 1.3-fold, at least about 1.4-fold, at least about 1.5-fold, at least about 1.6-fold, at least about 1.7-fold, at least about 1.8-fold, at least about 2-fold, at least about 2.5-fold, at least about 3-fold, at least about 3.5-fold, at least about 4-fold, at least about 4.5-fold, at least about 5-fold, at least about 5.5-fold, at least about 6-fold, at least about 6.5-fold, at least about 7-fold, at least about 7.5-fold, at least about 8-fold, at least about 8.5-fold, at least about 9-fold, at least about 9.5-fold, or at least about 10-fold or more.

### Kits, Research Reagents, Diagnostics, and Therapeutics

The compounds described herein can be utilized for diagnostics, therapeutics, and prophylaxis, and as research reagents and components of kits. Furthermore, antisense oligonucleotides, which are able to inhibit gene expression with exquisite specificity, are often used by those of ordinary skill to elucidate the function of particular genes or to distinguish between functions of various members of a biological pathway.

For use in kits and diagnostics and in various biological systems, the compounds described herein, either alone or in combination with other compounds or therapeutics, are useful as tools in differential and/or combinatorial analyses to elucidate expression patterns of a portion or the entire complement of genes expressed within cells and tissues.

As used herein the term “biological system” or “system” is defined as any organism, cell, cell culture or tissue that expresses, or is made competent to express products of the Arachidonate 15-lipoxygenase (ALOX15) genes. These include, but are not limited to, humans, transgenic animals, cells, cell cultures, tissues, xenografts, transplants and combinations thereof.

As one non limiting example, expression patterns within cells or tissues treated with one or more antisense compounds are compared to control cells or tissues not treated with antisense compounds and the patterns produced are analyzed for differential levels of gene expression as they pertain, for example, to disease association, signaling pathway, cellular localization, expression level, size, structure or function of the genes examined. These analyses can be performed on stimulated or unstimulated cells and in the presence or absence of other compounds that affect expression patterns.

Examples of methods of gene expression analysis known in the art include DNA arrays or microarrays, SAGE (serial analysis of gene expression), READS (restriction enzyme amplification of digested cDNAs), TOGA (total gene expression analysis), protein arrays and proteomics, expressed sequence tag (EST) sequencing, subtractive RNA fingerprinting (SuRF), subtractive cloning, differential display (DD), comparative genomic hybridization, FISH (fluorescent in situ hybridization) techniques and mass spectrometry methods.

The compounds described herein may be useful for research and diagnostics, because these compounds hybridize to nucleic acids encoding Arachidonate 15-lipoxygenase (ALOX15). For example, oligonucleotides that hybridize with such efficiency and under such conditions as disclosed herein as to be effective ALOX15 modulators are effective primers or probes under conditions favoring gene amplification or detection, respectively. These primers and probes are useful in methods requiring the specific detection of nucleic acid molecules encoding ALOX15 and in the amplification of said nucleic acid molecules for detection or for use in further studies of ALOX15. Hybridization of the antisense oligonucleotides, particularly the primers and probes, with a nucleic acid encoding ALOX15 can be detected by means known in the art. Such means may include conjugation of an enzyme to the oligonucleotide, radiolabeling of the oligonucleotide, or any other suitable detection means. Kits using such detection means for detecting the level of ALOX15 in a sample may also be prepared.

The specificity and sensitivity of antisense are also harnessed by those of skill in the art for therapeutic uses. Antisense compounds have been employed as therapeutic moieties in the treatment of disease states in animals, including humans. Antisense oligonucleotide drugs have been safely and effectively administered to humans and numerous clinical trials are presently underway. It is thus established that antisense compounds can be useful therapeutic modalities that can be configured to be useful in treatment regimens for the treatment of cells, tissues and animals, especially humans.

For therapeutics, an animal, e.g., a human, suspected of having a disease or disorder which can be treated by modulating the expression of ALOX15 polynucleotides is treated by administering antisense compounds. For example, in one non-limiting embodiment, the methods comprise the step of administering to the animal in need of treatment, a therapeutically effective amount of ALOX15 modulator. The ALOX15 modulators effectively modulate the activity of the ALOX15 or modulate the expression of the ALOX15 protein. In some embodiments, the activity or expression of ALOX15 in an animal is inhibited by about 10% as compared to a control. In some cases, the activity or expression of ALOX15 in an animal is inhibited by about 30%. In some cases, the activity or expression of ALOX15 in an animal is inhibited by 50% or more. Thus, the oligomeric compounds modulate expression of Arachidonate 15-lipoxygenase (ALOX15) mRNA by at least 10%, by at least 50%, by at least 25%, by at least 30%, by at least 40%, by at least 50%, by at least 60%, by at least 70%, by at least 75%, by at least 80%, by at least 85%, by at least 90%, by at least 95%, by at least 98%, by at least 99%, or by 100% as compared to a control.

For example, the reduction of the expression of Arachidonate 15-lipoxygenase, (ALOX15) may be measured in serum, blood, adipose tissue, liver or any other body fluid, tissue or organ of the animal. In some cases, the cells contained within said fluids, tissues or organs being analyzed contain a nucleic acid molecule encoding ALOX15 peptides and or the ALOX15 protein itself.

The compounds described herein can be utilized in pharmaceutical compositions by adding an effective amount of a compound to a suitable pharmaceutically acceptable diluent or carrier. Use of the compounds and methods described herein may also be useful prophylactically.

### SEQUENCES

Provided below are exemplary sequences, SEQ ID NOS: 1-39, related to one or more embodiments of the present disclosure.

TABLE 2
Sequences

| SEQ | | | |
|---|---|---|---|
| ID | | | |
| NO. | Description | Sequence | |
| 1 | Homo sapiens | 1 | GAGCGAAACA TCTTTGAGCA AGATGGGTCT CTACCGCATC CGCGTGTCCA CTGGGGCCTC |
| | arachidonate 15- | 61 | GCTCTATGCC GGTTCCAACA ACCAGGTGCA GCTGTGGCTG GTCGGCCAGC ACGGGGAGGC |
| | lipoxygenase, | 121 | GGCGCTCGGG AAGCGACTGT GGCCCGCACG GGGCAAGGAG ACAGAACTCA AGGTGGAAGT |
| | (ALOX15), mRNA | 181 | ACCGGAGTAT CTGGGGCCGC TGCTGTTTGT GAAACTGCGC AAACGGCACC TCCTTAAGGA |
| | (NCBI Accession | 241 | CGACGCCTGG TTCTGCAACT GGATCTCTGT GCAGGGCCCC GGAGCCGGGG ACGAGGTCAG |
| | No.: NM_001140.4) | 301 | GTTCCCTTGT TACCGCTGGG TGGAGGGCAA CGGCGTCCTG AGCCTGCCTG AAGGCACCGG |
| | | 361 | CCGCACTGTG GGCGAGGACC CTCAGGGCCT GTTCCAGAAA CACCGGGAAG AAGAGCTGGA |
| | | 421 | AGAGAGAAGG AAGTTGTACC GGTGGGGAAA CTGGAAGGAC GGGTTAATTC TGAATATGGC |
| | | 481 | TGGGGCCAAA CTATATGACC TCCCTGTGGA TGAGCGATTT CTGGAAGACA AGAGAGTTGA |
| | | 541 | CTTTGAGGTT TCGCTGGCCA AGGGGCTGGC CGACCTCGCT ATCAAAGACT CTCTAAATGT |
| | | 601 | TCTGACTTGC TGGAAGGATC TAGATGACTT CAACCGGATT TTCTGGTGTG GTCAGAGCAA |
| | | 661 | GCTGGCTGAG CGCGTGCGGG ACTCCTGGAA GGAAGATGCC TTATTTGGGT ACCAGTTTCT |
| | | 721 | TAATGGCGCC AACCCCGTGG TGCTGAGGCG CTCTGCTCAC CTTCCTGCTC GCCTAGTGTT |
| | | 781 | CCCTCCAGGC ATGGAGGAAC TGCAGGCCCA GCTGGAGAAG GAGCTGGAGG GAGGCACACT |
| | | 841 | GTTCGAAGCT GACTTCTCCC TGCTGGATGG GATCAAGGCC AACGTCATTC TCTGTAGCCA |
| | | 901 | GCAGCACCTG GCTGCCCCTC TAGTCATGCT GAAATTGCAG CCTGATGGGA AACTCTTGCC |
| | | 961 | CATGGTCATC CAGCTCCAGC TGCCCCGCAC AGGATCCCCA CCACCTCCCC TTTTCTTGCC |
| | | 1021 | TACGGATCCC CCAATGGCCT GGCTTCTGGC CAAATGCTGG GTGCGCAGCT CTGACTTCCA |
| | | 1081 | GCTCCATGAG CTGCAGTCTC ATCTTCTGAG GGGACACTTG ATGGCTGAGG TCATTGTTGT |
| | | 1141 | GGCCACCATG AGGTGCCTGC CGTCGATACA TCCTATCTTC AAGCTTATAA TTCCCCACCT |
| | | 1201 | GCGATACACC CTGGAAATTA ACGTCCGGGC CAGGACTGGG CTGGTCTCTG ACATGGGAAT |
| | | 1261 | TTTCGACCAG ATAATGAGCA CTGGTGGGGG AGGCCACGTG CAGCTGCTCA AGCAAGCTGG |
| | | 1321 | AGCCTTCCTA ACCTACAGCT CCTTCTGTCC CCCTGATGAC TTGGCCGACC GGGGGCTCCT |
| | | 1381 | GGGAGTGAAG TCTTCCTTCT ATGCCCAAGA TGCGCTGCGG CTCTGGGAAA TCATCTATCG |
| | | 1441 | GTATGTGGAA GGAATCGTGA GTCTCCACTA TAAGACAGAC GTGGCTGTGA AAGACGACCC |
| | | 1501 | AGAGCTGCAG ACCTGGTGTC GAGAGATCAC TGAAATCGGG CTGCAAGGGG CCCAGGACCG |
| | | 1561 | AGGGTTTCCT GTCTCTTTAC AGGCTCGGGA CCAGGTTTGC CACTTTGTCA CCATGTGTAT |
| | | 1621 | CTTCACCTGC ACCGGCCAAC ACGCCTCTGT GCACCTGGGC CAGCTGGACT GGTACTCTTG |
| | | 1681 | GGTGCCTAAT GCACCCTGCA CGATGCGGCT GCCCCCGCCA ACCACCAAGG ATGCAACGCT |
| | | 1741 | GGAGACAGTG ATGGCGACAC TGCCCAACTT CCACCAGGCT TCTCTCCAGA TGTCCATCAC |
| | | 1801 | TTGGCAGCTG GGCAGACGCC AGCCCGTTAT GGTGGCTGTG GGCCAGCATG AGGAGGAGTA |
| | | 1861 | TTTTTCGGGC CCTGAGCCTA AGGCTGTGCT GAAGAAGTTC AGGGAGGAGC TGGCTGCCCT |
| | | 1921 | GGATAAGGAA ATTGAGATCC GGAATGCAAA GCTGGACATG CCCTACGAGT ACCTGCGGCC |
| | | 1981 | CAGCGTGGTG GAAAACAGTG TGGCCATCTA AGCGTCGCCA CCCTTTGGTT ATTTCAGCCC |
| | | 2041 | CCATCACCCA AGCCACAAGC TGACCCCTTC GTGGTTATAG CCCTGCCCTC CCAAGTCCCA |
| | | 2101 | CCCTCTTCCC ATGTCCCACC CTCCCTAGAG GGGCACCTTT TCATGGTCTC TGCACCCAGT |
| | | 2161 | GAACACATTT TACTCTAGAG GCATCACCTG GGACCTTACT CCTCTTTCCT TCCTTCCTCC |
| | | 2221 | TTTCCTATCT TCCTTCCTCT CTCTCTTCCT CTTTCTTCAT TCAGATCTAT ATGGCAAATA |
| | | 2281 | GCCACAATTA TATAAATCAT TTCAAGACTA GAATAGGGGG ATATAATACA TATTACTCCA |
| | | 2341 | CACCTTTTAT GAATCAAATA TGATTTTTTT GTTGTTGTTA AGACAGAGTC TCACTTTGAC |
| | | 2401 | ACCCAGGCTG GAGTGCAGTG GTGCCATCAC CACGGCTCAC TGCAGCCTCA GCGTCCTGGG |
| | | 2461 | CTCAAATGAT CCTCCCACCT CAGCCTCCTG AGTAGCTGGG ACTACAGGCT CATGCCATCA |
| | | 2521 | TGCCCAGCTA ATATTTTTTT ATTTTCGTGG AGACGGGGCC TCACTATGTT GCCTAGGCTG |
| | | 2581 | GAAATAGGAT TTTGAACCCA AATTGAGTTT AACAATAATA AAAAGTTGTT TTACGCTAAA |
| | | 2641 | GATGGAAAAG AACTAGGACT GAACTATTTT AAATAAAATA TTGGCAAAAG AAAAAAAAAA |
| | | 2701 | AAAAAAAAAA AAAAA |

| | | |
|---|---|---|
| 2 | Homo sapiens | TTTTTTTTTTTTTTTTTTTTTTTTTCTTTTGCCAATATTTT |
| | arachidonate 15- | ATTTAAAATAGTTCAGTCCTAGTTCTTTTCCATCTTTAG |
| | lipoxygenase | CGTAAAACAACTTTTTATTATTGTTAAACTCAATTTGGG |
| | (ALOX15), mRNA, | TTCAAAATCCTATTTCCAGCCTAGGCAACATAGTGAGG |
| | reverse complement | CCCCGTCTCCACGAAAATAAAAAAATATTAGCTGGGCA |
| | | TGATGGCATGAGCCTGTAGTCCCAGCTACTCAGGAGGC |
| | | TGAGGTGGGAGGATCATTTGAGCCCAGGACGCTGAGG |
| | | CTGCAGTGAGCCGTGGTGATGGCACCACTGCACTCCAG |
| | | CCTGGGTGTCAAAGTGAGACTCTGTCTTAACAACAACA |
| | | AAAAAATCATATTTGATTCATAAAAGGTGTGGAGTAAT |
| | | ATGTATTATATCCCCCTATTCTAGTCTTGAAATGATTTA |
| | | TATAATTGTGGCTATTTGCCATATAGATCTGAATGAAG |
| | | AAAGAGGAAGAGAGAGAGGAAGGAAGATAGGAAAGG |
| | | AGGAAGGAAGGAAAGAGGAGTAAGGTCCCAGGTGATG |
| | | CCTCTAGAGTAAAATGTGTTCACTGGGTGCAGAGACCA |
| | | TGAAAAGGTGCCCCTCTAGGGAGGGTGGGACATGGGA |
| | | AGAGGGTGGGACTTGGGAGGGCAGGGCTATAACCACG |
| | | AAGGGGTCAGCTTGTGGCTTGGGTGATGGGGGCTGAA |
| | | ATAACCAAAGGGTGGCGACGCTTAGATGGCCACACTGT |
| | | TTTCCACCACGCTGGGCCGCAGGTACTCGTAGGGCATG |
| | | TCCAGCTTTGCATTCCGGATCTCAATTTCCTTATCCAGG |
| | | GCAGCCAGCTCCTCCCTGAACTTCTTCAGCACAGCCTT |
| | | AGGCTCAGGGCCCGAAAAATACTCCTCCTCATGCTGGC |
| | | CCACAGCCACCATAACGGGCTGGCGTCTGCCCAGCTGC |
| | | CAAGTGATGGACATCTGGAGAGAAGCCTGGTGGAAGT |
| | | TGGGCAGTGTCGCCATCACTGTCTCCAGCGTTGCATCC |
| | | TTGGTGGTTGGCGGGGGCAGCCGCATCGTGCAGGGTGC |
| | | ATTAGGCACCCAAGAGTACCAGTCCAGCTGGCCCAGGT |
| | | GCACAGAGGCGTGTTGGCCGGTGCAGGTGAAGATACA |
| | | CATGGTGACAAAGTGGCAAACCTGGTCCCGAGCCTGTA |
| | | AAGAGACAGGAAACCCTCGGTCCTGGGCCCCTTGCAGC |
| | | CCGATTTCAGTGATCTCTCGACACCAGGTCTGCAGCTC |
| | | TGGGTCGTCTTTCACAGCCACGTCTGTCTTATAGTGGA |
| | | GACTCACGATTCCTTCCACATACCGATAGATGATTTCC |
| | | CAGAGCCGCAGCGCATCTTGGGCATAGAAGGAAGACT |
| | | TCACTCCCAGGAGCCCCCGGTCGGCCAAGTCATCAGGG |
| | | GGACAGAAGGAGCTGTAGGTTAGGAAGGCTCCAGCTT |
| | | GCTTGAGCAGCTGCACGTGGCCTCCCCCACCAGTGCTC |
| | | ATTATCTGGTCGAAAATTCCCATGTCAGAGACCAGCCC |
| | | AGTCCTGGCCCGGACGTTAATTTCCAGGGTGTATCGCA |
| | | GGTGGGGAATTATAAGCTTGAAGATAGGATGTATCGAC |
| | | GGCAGGCACCTCATGGTGGCCACAACAATGACCTCAGC |
| | | CATCAAGTGTCCCCTCAGAAGATGAGACTGCAGCTCAT |
| | | GGAGCTGGAAGTCAGAGCTGCGCACCCAGCATTTGGCC |
| | | AGAAGCCAGGCCATTGGGGGATCCGTAGGCAAGAAAA |
| | | GGGGAGGTGGTGGGGATCCTGTGCGGGGCAGCTGGAG |
| | | CTGGATGACCATGGGCAAGAGTTTCCCATCAGGCTGCA |
| | | ATTTCAGCATGACTAGAGGGGCAGCCAGGTGCTGCTGG |
| | | CTACAGAGAATGACGTTGGCCTTGATCCCATCCAGCAG |
| | | GGAGAAGTCAGCTTCGAACAGTGTGCCTCCCTCCAGCT |
| | | CCTTCTCCAGCTGGGCCTGCAGTTCCTCCATGCCTGGA |
| | | GGGAACACTAGGCGAGCAGGAAGGTGAGCAGAGCGCC |
| | | TCAGCACCACGGGGTTGGCGCCATTAAGAAACTGGTAC |
| | | CCAAATAAGGCATCTTCCTTCCAGGAGTCCCGCACGCG |
| | | CTCAGCCAGCTTGCTCTGACCACACCAGAAAATCCGGT |
| | | TGAAGTCATCTAGATCCTTCCAGCAAGTCAGAACATTT |
| | | AGAGAGTCTTTGATAGCGAGGTCGGCCAGCCCCTTGGC |
| | | CAGCGAAACCTCAAAGTCAACTCTCTTGTCTTCCAGAA |
| | | ATCGCTCATCCACAGGGAGGTCATATAGTTTGGCCCCA |
| | | GCCATATTCAGAATTAACCCGTCCTTCCAGTTTCCCCAC |
| | | CGGTACAACTTCCTTCTCTCTTCCAGCTCTTCTTCCCGG |
| | | TGTTTCTGGAACAGGCCCTGAGGGTCCTCGCCCACAGT |
| | | GCGGCCGGTGCCTTCAGGCAGGCTCAGGACGCCGTTGC |
| | | CCTCCACCCAGCGGTAACAAGGGAACCTGACCTCGTCC |
| | | CCGGCTCCGGGGCCCTGCACAGAGATCCAGTTGCAGAA |
| | | CCAGGCGTCGTCCTTAAGGAGGTGCCGTTTGCGCAGTT |
| | | TCACAAACAGCAGCGGCCCCAGATACTCCGGTACTTCC |
| | | ACCTTGAGTTCTGTCTCCTTGCCCCGTGCGGGCCACAGT |
| | | CGCTTCCCGAGCGCCGCCTCCCCGTGCTGGCCGACCAG |
| | | CCACAGCTGCACCTGGTTGTTGGAACCGGCATAGAGCG |
| | | AGGCCCCAGTGGACACGCGGATGCGGTAGAGACCCAT |
| | | CTTGCTCAAAGATGTTTCGCTC |
| 3 | human and mouse | CTGAACTTCTTCAGCACAGC |
| | cross-reactive | |
| | ALOX15 antisense | |
| | oligonucleotide | |
| 4 | control antisense | TCTAACCGAGCTGATGGACT |
| | oligonucleotide | |
| 5 | human and rabbit | CAGAAATCGCTCATCCACAG |
| | cross-reactive | |
| | ALOX15 antisense | |
| | oligonucleotide | |
| 6 | dsRNA 701a sense | GGAGUACACGUUCCCCUGUUAdTdT |
| | strand | |
| 7 | dsRNA 701a | UAACAGGGGAACGUGUACUCCdTdT |
| | antisense strand | |
| 8 | dsRNA 701a ctl | GUCCAUCAGCUCGGUUAGACUdTdT |
| | sense strand | |
| 9 | dsRNA 701a ctl | AGUCUAACCGAGCUGAUGGACdTdT |
| | antisense strand | |
| 10 | dsRNA 701b sense | GGUGGAAGUACCGGAGUAUCUdTdT |
| | strand | |
| 11 | dsRNA 701b | AGAUACUCCGGUACUUCCACCdTdT |
| | antisense strand | |
| 12 | dsRNA 701b ctl | GUUGUACAGCAUGCGGAGAGUdTdT |
| | sense strand | |
| 13 | dsRNA 701b ctl | ACUCUCCGCAUGCUGUACAACdTdT |
| | antisense strand | |
| 14 | dsRNA sense strand | mGsmCmUmGfUmGmCmUfGmAmAmGfAmAmGmUfUfCm |
| | 3766a | AsdTsdT |
| 15 | dsRNA antisense | mUsfGmAmAmCfUfUmCmUfUfCfAmGfCmAmCmAfGm |
| | strand 3767a | CsdTsdT |
| 16 | dsRNA sense strand | GCUGUGCUGAAGAAGUUCAdTdT |
| | 3766c | |
| 17 | dsRNA antisense | UGAACUUCUUCAGCACAGCdTdT |
| | strand 3767c | |
| 18 | dsRNA sense strand | GUCCAUCAGCUCGGUUAGAdTdT |
| | 3766d | |
| 19 | dsRNA antisense | UCUAACCGAGCUGAUGGACdTdT |
| | strand 3767d | |
| 20 | dsRNA sense strand | mCsmUmGmUfGmGmAmUfGmAmGmCfGmAmUmUfUfCm |
| | 1008a | UsdTsdT |
| 21 | dsRNA antisense | mAsfGmAmAmAfUfCmGmCfUfCfAmUfCmCmAmCfAmGsd |
| | strand 1009a | TsdT |
| 22 | dsRNA control sense | mGsmUmCmCfAmUmCmAmfGmCmUmCmfGmGmUmUfAf |
| | strand a | GmAsdTsdT |
| 23 | dsRNA control | mUsfCmUmAmAfCfCmGmAfGfCfUmGfAmUmGmGfAmCdT |
| | antisense strand a | dT |
| 24 | Antisense oligo 3 | mCsmTsmGsmAsmAsdCsdTsdTsdCsdTsdTsdCsdAsdGsdCsm |
| | | AsmCsmAsmGsmC |
| 25 | Antisense oligo 4 | mTsmCsmTsmAsmAsdCsdCsdGsdAsdGsdCsdTsdGsdAsdTsm |
| | | GsmGsmAsmCsmT |
| 26 | RFGF peptide | AAVALLPAVLLALLAP |
| 27 | RFGF analogue | AALLPVLLAAP |
| | peptide | |
| 28 | Human | GRKKRRQRRRPPQ |
| | immunodeficiency | |
| | virus | |
| 29 | Drosophila sp. | RQIKIWFQNRRMKWK |
| 30 | Synthetic | gcugugcugaagaaguucatt |
| | oligonucleotide 1 | |
| 31 | Synthetic | ugaacuucuucagcacagctt |
| | oligonucleotide 2 | |
| 32 | Synthetic | guccaucagcucgguuagatt |
| | oligonucleotide 3 | |
| 33 | Synthetic | ucuaaccgagcugauggactt |
| | oligonucleotide 4 | |
| 34 | Synthetic | cuguggaugagcgauuucutt |
| | oligonucleotide 5 | |
| 35 | Synthetic | agaaaucgcucauccacagtt |
| | oligonucleotide 6 | |
| 36 | siRNA s1288 sense | GGAAAUCAUCUAUCGGUAUtt |
| | strand | |
| 37 | siRNA s1288 | AUACCGAUAGAUGAUUUCCtt |
| | antisense strand | |
| 38 | siRNA s1289 sense | GCACACUGUUCGAAGCUGAtt |
| | strand | |
| 39 | siRNA s1289 | UCAGCUUCGAACAGUGUGCtt |
| | antisense strand | |

### FURTHER EMBODIMENTS

1. A method of modulating a function of an Arachidonate 15-lipoxygenase in mammalian cells or tissues in vivo and in vitro comprising: contacting said cells or tissues with at least one molecule disclosed herein, wherein said at least one molecule modulates the 15-lipoxygenase function in mammalian cells or tissues in vivo or in vitro.

2. A method of altering expression of an Arachidonate 15-lipoxygenase (ALOX15) polynucleotide in mammalian cells or tissues in vivo or in vitro comprising: contacting said cells or tissues with at least one antisense oligonucleotide 5 to 30 nucleotides in length wherein said at least one oligonucleotide has at least 50% sequence identity to a reverse complement of a polynucleotide comprising 5 to 30 consecutive nucleotides within nucleotides 1 to 2715 of SEQ ID NO: 2; thereby altering the expression of the Arachidonate 15-lipoxygenase (ALOX15) polynucleotide in mammalian cells or tissues in vivo or in vitro.

3. A double-stranded RNAi agent capable of inhibiting the expression of a target gene, comprising a sense strand and an antisense strand, each strand having 14 to 30 nucleotides.

4. The double-stranded RNAi agent of embodiment 3, wherein the duplex region is 17-30 nucleotide pairs in length.

5. The double-stranded RNAi agent of embodiment 3 or 4, wherein each strand has 17-30 nucleotides.

6. The double-stranded RNAi agent of any one of embodiments 3-5, wherein the sense strand consists of SEQ ID NO: 16 and the antisense strand consists of SEQ ID NO: 17.

7. The double-stranded RNAi agent of any one of embodiments 3-6, wherein the modifications on the nucleotides are selected from the group consisting of LNA, HNA, CeNA, 2′-methoxyethyl, 2′-0-alkyl, 2′-0-allyl, 2′-C-allyl, 2′-fluoro, 2′-deoxy, and combinations thereof.

8. The double-stranded RNAi agent of any one of embodiments 3-7, wherein the nucleotides are modified with either 2′-OCH3 or 2′-F.

9. The double-stranded RNAi agent of any one of embodiments 3-8, wherein the double-stranded RNAi agent further comprises at least one ligand.

10. The double-stranded RNAi agent of any one of embodiments 3-9, wherein the modifications on the nucleotides are selected from the group consisting of 2′-0-methyl nucleotide, 2′-deoxyfluoro nucleotide, 2′-0-N-methylacetamido (2′-0-NMA) nucleotide, a 2′-0-dimethylaminoethoxyethyl (2′-0-DMAEOE) nucleotide, 2′-0-aminopropyl (2′-0-AP) nucleotide, 2′-ara-F, and combinations thereof.

11. The double-stranded RNAi agent of any one of embodiments 3-10, wherein the double-stranded RNAi agent further comprises at least one phosphorothioate or methylphosphonate internucleotide linkage.

12. The double-stranded RNAi agent of any one of embodiments 3-11, wherein the nucleotide at the 1 position of the 5′-end of the duplex in the antisense strand is selected from the group consisting of A, dA, dU, U, and dT.

13. The double-stranded RNAi agent of any one of embodiments 3-12, wherein the base pair at the 1 position of the 5′-end of the duplex is an AU base pair.

14. A double-stranded RNAi agent capable of inhibiting the expression of a target gene, comprising a sense strand and an antisense strand, each strand having 14 to 30 nucleotides, wherein the sense strand contains at least two motifs of three identical modifications on three consecutive nucleotides, one of said motifs occurring at the cleavage site in the strand and at least one of said motifs occurring at another portion of the strand that is separated from the motif at the cleavage site by at least one nucleotide; and wherein the antisense strand contains at least first motif of three identical modifications on three consecutive nucleotides, one of said motifs occurring at or near the cleavage site in the strand and a second motif occurring at another portion of the strand that is separated from the first motif by at least one nucleotide; wherein the modification in the motif occurring at the cleavage site in the sense strand is different than the modification in the motif occurring at or near the cleavage site in the antisense strand.

15. The double-stranded RNAi agent of embodiment 14, wherein at least one of the nucleotides occurring at the cleavage site in the sense strand forms a base pair with one of the nucleotides in the motif occurring at or near the cleavage site in the antisense strand.

16. The double-stranded RNAi agent of embodiment 14 or 15, wherein the duplex has 17-30 nucleotides.

17. The double-stranded RNAi agent of any one of embodiments 14-16, wherein the duplex has 17-19 nucleotides.

18. The double-stranded RNAi agent of any one of embodiments 14-17, wherein each strand has 17-23 nucleotides.

19. The double-stranded RNAi agent of any one of embodiments 14-18, wherein the modifications on the nucleotides are selected from the group consisting of LNA, HNA, CeNA, 2′-methoxyethyl, 2′-O-alkyl, 2′-O-allyl, 2′-C-allyl, 2′-fluoro, 2′-deoxy, and combinations thereof.

20. The double-stranded RNAi agent of any one of embodiments 14-19, wherein the modifications on the nucleotide are 2′-OCH3 or 2′-F.

21. The double-stranded RNAi agent of any one of embodiments 14-20, wherein the double-stranded RNAi agent further comprises a ligand attached to the 3′ end of the sense strand.

22. A double-stranded RNAi agent capable of inhibiting the expression of a target gene, comprising a sense strand and an antisense strand, each strand having 14 to 30 nucleotides, wherein the sense strand contains at least one motif of three 2′-F modifications on three consecutive nucleotides, one of said motifs occurring at or near the cleavage site in the strand; and wherein the antisense strand contains at least one motif of three 2′-O-methyl modifications on three consecutive nucleotides, one of said motifs occurring at or near the cleavage site.

23. A method of treating one or more disorders of the upper and lower airway in a subject in need thereof comprising administering to the subject an inhibitor of arachidonate 15-lipoxygenase (ALOX15) wherein the one or more disorders of the upper and lower airway comprises nasal polyposis, chronic sinusitis, allergic rhinitis, asthma, or NSAID-exacerbated respiratory disease.

24. The method of embodiment 23, wherein the inhibitor of ALOX15 is delivered systemically to the subject.

25. The method of embodiment 23, wherein the inhibitor of ALOX15 is delivered locally to the subject.

26. The method of embodiment 23, wherein the inhibitor of ALOX15 is delivered locally to the nasal epithelium of the subject.

27. The method of any one of embodiments 23-26, wherein the one or more disorders of the upper and lower airway is nasal polyposis.

28. The method of any one of embodiments 23-27, wherein the subject has nasal polyps.

29. The method of embodiment 28, wherein tissue from the subject comprising the nasal polyps comprises eosinophilic infiltration.

30. The method of any one of embodiments 23-29, wherein the subject has received a first line treatment comprising intranasal corticosteroids for the one or more disorders of the upper and lower airway.

31. The method of any one of embodiments 23-30, wherein the inhibitor of ALOX15 comprises a small molecule.

32. The method of any one of embodiments 23-30, wherein the inhibitor of ALOX15 comprises RNAi.

33. The method of embodiment 32, wherein the RNAi inhibits translation or degrades ALOX15 mRNA.

34. The method of embodiment 32 or 33, wherein the RNAi comprises siRNA, miRNA, or antisense oligonucleotide (ASO).

35. The method of embodiment 34, wherein the ASO is single-stranded or double-stranded.

36. The method of any one of embodiments 23-30, wherein the inhibitor of ALOX15 is an aptamer.

37. The method of embodiment 36, wherein the aptamer is an oligonucleotide or a peptide molecule.

38. The method of any one of embodiments 23-37, wherein the subject comprises an ALOX15 variant.

39. The method of embodiment 38, wherein the ALOX15 variant is rs2255888.

40. The method of any one of embodiments 23-39, wherein the inhibitor of ALOX15 causes a reduction in the production of a metabolite of ALOX15 in the subject.

41. The method of embodiment 40, wherein the metabolite of ALOX15 is 15-hydroxyeicosatetraenoic acid (15-HETE).

42. The method of any one of embodiments 23-41, wherein the inhibitor of ALOX15 causes a reduction in the subject of blood eosinophil counts.

43. A composition comprising an inhibitor of ALOX15 that is efficacious in treating nasal polyposis, chronic sinusitis, allergic rhinitis, asthma, or NSAID-exacerbated respiratory disease.

44. The composition of embodiment 43, wherein the inhibitor of ALOX15 is an RNAi.

45. The composition of embodiment 44, wherein the RNAi is siRNA.

46. The composition of embodiment 44, wherein the RNAi is miRNA.

47. The composition of embodiment 44, wherein the RNAi is an antisense oligonucleotide (ASO).

48. The composition of embodiment 47, wherein the ASO is double-stranded or single-stranded.

49. The composition of embodiment 43, wherein the inhibitor of ALOX15 is a small molecule.

50. The composition of embodiment 43, wherein the inhibitor of ALOX15 is an aptamer.

51. The composition of embodiment 43, wherein the aptamer is an oligonucleotide aptamer.

52. The composition of embodiment 51, wherein the aptamer is a peptide aptamer.

53. A method of treating one or more disorders of the upper and lower airway in a subject in need thereof comprising editing an ALOX15 gene in the subject wherein the one or more disorders of the upper and lower airway comprises nasal polyposis, chronic sinusitis, allergic rhinitis, asthma, or NSAID-exacerbated respiratory disease.

54. The method of embodiment 53, wherein the editing of the ALOX15 gene comprises administering CRISPR/cas9 to the subject.

55. The method of embodiment 54, wherein the CRISPR/cas9 targets the ALOX15 gene.

56. The method of embodiment 54 or 55, wherein the CRISPR/cas9 edits the ALOX15 gene to a loss of function mutation.

57. The method of embodiment 56, wherein the loss of function mutation comprises a threonine to methionine mutation.

58. The method of embodiment 57, wherein the threonine to methionine mutation occurs at amino acid position 560 according to the human protein sequence numbering.

59. The method of any one of embodiments 54-58, wherein the CRISPR/cas9 is delivered systemically to the subject.

60. The method of any one of embodiments 54-58, wherein the CRISPR/cas9 is delivered locally to the subject.

61. The method of any one of embodiments 54-58, wherein the CRISPR/cas9 is delivered locally to the nasal epithelium of the subject.

62. The method of any one of embodiments 53-61, wherein the editing of the ALOX15 gene is efficacious in treating the one or more disorders of the upper and lower airway.

63. The method of any one of embodiments 53-62, wherein the one or more disorders of the upper and lower airway is nasal polyposis.

64. The method of any one of embodiments 53-63, wherein the subject has nasal polyps.

65. The method of embodiment 64, wherein tissue from the subject comprising the nasal polyps comprises eosinophilic infiltration.

66. The method of any one of embodiments 53-65, wherein the subject has received a first line treatment comprising intranasal corticosteroids for the one or more disorders of the upper and lower airway.

67. The method of any one of embodiments 53-66, wherein the editing of the ALOX15 gene causes a reduction in the production of a metabolite of ALOX15 in the subject.

68. The method of embodiment 67, wherein the metabolite of ALOX15 is 15-hydroxyeicosatetraenoic acid (15-HETE).

69. The method of any one of embodiments 53-68, wherein the editing of the ALOX15 gene causes a reduction in the subject of blood eosinophil counts.

70. A composition comprising CRISPR/cas9 that targets ALOX15 that is efficacious in treating nasal polyposis, chronic sinusitis, allergic rhinitis, asthma, or NSAID-exacerbated respiratory disease.

71. The composition of embodiment 70, wherein the CRISPR/cas9 edits the ALOX15 gene to a loss of function mutation.

72. The composition of embodiment 71, wherein the loss of function mutation comprises a threonine to methionine mutation.

73. The composition of embodiment 72, wherein the threonine to methionine mutation occurs at amino acid position 560 according to the human protein sequence numbering.

74. A method of treating one or more disorders of the gastrointestinal tract in a subject in need thereof comprising administering to the subject an inhibitor of arachidonate 15-lipoxygenase (ALOX15) wherein the one or more disorders of the gastrointestinal tract comprises eosinophilic esophagitis, eosinophilic gastritis, or eosinophilic colitis.

75. The method of embodiment 74, wherein the inhibitor of ALOX15 is delivered systemically to the subject.

76. The method of embodiment 74, wherein the inhibitor of ALOX15 is delivered locally to the subject.

77. The method of any one of embodiments 74-76, wherein the one or more disorders of the gastrointestinal tract is eosinophilic esophagitis.

78. The method of any one of embodiments 74-77, wherein tissue from the subject comprising the eosinophilic esophagitis comprises eosinophilic infiltration.

79. The method of any one of embodiments 74-78, wherein the subject has received a first line treatment comprising systemic corticosteroids for the one or more disorders of the gastrointestinal tract.

80. The method of any one of embodiments 74-79, wherein the inhibitor of ALOX15 comprises a small molecule.

81. The method of any one of embodiments 74-79, wherein the inhibitor of ALOX15 comprises RNAi.

82. The method of embodiment 81, wherein the RNAi inhibits translation or degrades ALOX15 mRNA.

83. The method of embodiment 81, wherein the RNAi comprises siRNA, miRNA, or antisense oligonucleotide (ASO).

84. The method of embodiment 83, wherein the ASO is single-stranded or double-stranded.

85. The method of any one of embodiments 74-79, wherein the inhibitor of ALOX15 is an aptamer.

86. The method of embodiment 85, wherein the aptamer is an oligonucleotide or a peptide molecule.

87. The method of any one of embodiments 74-86, wherein the subject comprises an ALOX15 variant.

88. The method of embodiment 87, wherein the ALOX15 variant is rs2255888.

89. The method of any one of embodiments 74-88, wherein the inhibitor of ALOX15 causes a reduction in the production of a metabolite of ALOX15 in the subject.

90. The method of embodiment 89, wherein the metabolite of ALOX15 is 15-hydroxyeicosatetraenoic acid (15-HETE).

91. The method of any one of embodiments 74-90, wherein the inhibitor of ALOX15 causes a reduction in the subject of blood eosinophil counts.

92. A composition comprising an inhibitor of ALOX15 that is efficacious in treating eosinophilic esophagitis, eosinophilic gastritis, or eosinophilic colitis.

93. The composition of embodiment 92, wherein the inhibitor of ALOX15 is an RNAi.

94. The composition of embodiment 93, wherein the RNAi is siRNA.

95. The composition of embodiment 93, wherein the RNAi is miRNA.

96. The composition of embodiment 93, wherein the RNAi is an antisense oligonucleotide (ASO).

97. The composition of embodiment 96, wherein the ASO is double-stranded or single-stranded.

98. The composition of any one of embodiments 92-97, wherein the inhibitor of ALOX15 is a small molecule.

99. The composition of any one of embodiments 92-97, wherein the inhibitor of ALOX15 is an aptamer.

100. The composition of embodiment 99, wherein the aptamer is an oligonucleotide aptamer.

101. The composition of embodiment 99, wherein the aptamer is a peptide aptamer.

102. A method of treating one or more disorders of the gastrointestinal tract in a subject in need thereof comprising editing an ALOX15 gene in the subject wherein the one or more disorders of the gastrointestinal tract comprises eosinophilic esophagitis, eosinophilic gastritis, or eosinophilic colitis.

103. The method of embodiment 102, wherein the editing of the ALOX15 gene comprises administering CRISPR/cas9 to the subject.

104. The method of embodiment 103, wherein the CRISPR/cas9 targets the ALOX15 gene.

105. The method of embodiment 103 or 104, wherein the CRISPR/cas9 edits the ALOX15 gene to a loss of function mutation.

106. The method of embodiment 105, wherein the loss of function mutation comprises a threonine to methionine mutation.

107. The method of embodiment 106, wherein the threonine to methionine mutation occurs at amino acid position 560 according to the human protein sequence numbering.

108. The method of any one of embodiments 103-107, wherein the CRISPR/cas9 is delivered systemically to the subject.

109. The method of any one of embodiments 103-107, wherein the CRISPR/cas9 is delivered locally to the subject.

110. The method of any one of embodiments 102-109, wherein the editing of the ALOX15 gene is efficacious in treating the one or more disorders of the gastrointestinal tract.

111. The method of any one of embodiments 102-110, wherein the one or more disorders of the gastrointestinal tract airway is eosinophilic esophagitis.

112. The method of embodiment 111, wherein tissue from the subject comprising the eosinophilic esophagitis comprises eosinophilic infiltration.

113. The method of any one of embodiments 102-112, wherein the subject has received a first line treatment comprising systemic corticosteroids for the one or more disorders of the gastrointestinal tract.

114. The method of any one of embodiments 102-113, wherein the editing of the ALOX15 gene causes a reduction in the production of a metabolite of ALOX15 in the subject.

115. The method of embodiment 114, wherein the metabolite of ALOX15 is 15-hydroxyeicosatetraenoic acid (15-HETE).

116. The method of any one of embodiments 102-115, wherein the editing of the ALOX15 gene causes a reduction in the subject of blood eosinophil counts.

117. A composition comprising CRISPR/cas9 that targets ALOX15 that is efficacious in treating eosinophilic esophagitis, eosinophilic gastritis, or eosinophilic colitis.

118. The composition of embodiment 117, wherein the CRISPR/cas9 edits the ALOX15 gene to a loss of function mutation.

119. The composition of embodiment 118, wherein the loss of function mutation comprises a threonine to methionine mutation.

120. The composition of embodiment 119, wherein the threonine to methionine mutation occurs at amino acid position 560 according to the human protein sequence numbering.

The present disclosure is further illustrated by the following examples, which should not be construed as further limiting. The contents of all references, pending patent applications and published patents, cited throughout this application are hereby expressly incorporated by reference.

### EXAMPLES

### Example 1: Determination of Genomic Loci Associated with Eosinophilic Airway Disease Traits

Approximately 30,000,000 imputed and directly genotyped variants in 4,125 individuals with a diagnosis of nasal polyposis and 319,054 controls without a diagnosis of nasal polyposis were evaluated in the UK Biobank cohort. Logistic regression adjusting for age and sex was carried out, and adjusted for population substructure using principal components as covariates. Applicant discovered about 20 genomic loci significantly associated with diagnosis of nasal polyposis, which are represented in a Manhattan plot in FIG. 2.

One cluster of association was at chromosome 17p13.2, encompassing the ALOX15 gene. The most significantly associated variant (rs34210653) at this locus was a low frequency missense variant (minor allele frequency˜1.7%) in exon 13 of the ALOX15 gene which was consistently associated with the evaluated phenotypes (Table 3). This variant was associated with reduced risk of nasal polyposis; carriers of the minor allele of this variant had less than half the risk of nasal polyposis as non-carriers (p=2×10\hat{ }-15; OR=0.38). This variant was also associated with reduced risk of chronic rhinosinusitis (p=7×10\hat{ }-12; OR=0.65), allergic rhinitis (p=5×10\hat{ }-9; OR=0.80), asthma (p=9×10\hat{ }-6; OR=0.93) and reduced risk of undergoing sinus surgery including nasal polypectomy (p=5×10\hat{ }-11; OR=0.46). This variant was also associated with reduced blood eosinophil counts (p=2×10\hat{ }-65; beta=−0.02).

Table 3. Association of ALOX15 Variant rs34210653 with Disease Traits

TABLE 3
Association of ALOX15 Variant rs34210653 with Disease Traits
ALOX15 rs34210653 (AAF = 0.017)

| | Case | Control | Effect | | P |
|---|---|---|---|---|---|
| Phenotype | Count | Count | Size | | Value |
| Nasal Polyposis | 4,125 | 319,054 | 0.38 | (OR) | 1.8E−15 |
| Chronic | 9,535 | 319,054 | 0.65 | (OR) | 7.2E−12 |
| Rhinosinusitis | | | | | |
| Sinus Surgery | 3,592 | 319,054 | 0.46 | (OR) | 5.3E−11 |
| Allergic Rhinitis | 22,509 | 259,537 | 0.80 | (OR) | 4.5E−09 |
| Asthma | 41,307 | 281,367 | 0.93 | (OR) | 9.1E−06 |
| Eosinophil | 324,572 | NA | −0.02 | (Beta) | 1.5E−65 |
| Count | | | | | |

ALOX15 is one of five (ALOX5/12/12B/15/15B) human lipoxygenases and is involved in the metabolism of arachidonic acid and other polyunsaturated fatty acid substrates (FIG. 3). 15-HETE is the major metabolite of ALOX15 mediated arachidonic acid metabolism; 15-HETE is further metabolized to a number of molecules which mediate inflammation, including lipoxins and eoxins.

The rs34210653 variant results in a threonine to methionine change at amino acid 560 (T560M). Reports demonstrate that this T560M exchange results in near complete ablation of ALOX15 catalytic activity (FIG. 4), as measured by 15-HETE production from arachidonic acid.

By inference, these data indicate that loss of function of ALOX15 protects against the development of nasal polyps, chronic rhinosinusitis, allergic rhinitis and asthma and strongly suggest therapeutic inhibition of ALOX15 as a novel, genetically-informed method of treatment for these diseases.

In addition to the protective loss of function variant rs34210653, Applicant discovered that a separate, independent ALOX15 variant is associated with risk of these diseases. This variant, rs2255888, is an ALOX15 regulatory variant that is associated with increased expression of ALOX15 in whole blood (FIG. 5). The data used for the analyses presented in FIG. 5 were obtained from: the GTEx Portal (Genotype-Tissue Expression Project, supported by the Common Fund of the Office of the Director of the National Institutes of Health, and by NCI, NHGRI, NHLBI, NIDA, NIMH, and NINDS) on Feb. 1, 2018.

In an analysis conditioning on the LOF variant, the T allele of rs2255888, which is associated with increased expression of ALOX15 in whole blood, is associated with increased risk of nasal polyposis (p=7×10\hat{ }-5; OR=1.2) and increased blood eosinophil counts (p=2×10\hat{ }-22; beta=0.004).

In combination with the T560M loss of function variant, Applicant has therefore identified an ALOX15 allelic series that modulates risk for nasal polyposis and blood eosinophils. This allelic series consist of an ALOX15 loss of function variant (rs34210653) that is associated with decreased risk of nasal polyposis, chronic rhinosinusitis, allergic rhinitis, asthma and decreased blood eosinophil counts, and an ALOX15 regulatory variant that increases ALOX15 expression (rs2255888) that is associated with increased risk of nasal polyposis and increased blood eosinophil counts, further suggesting therapeutic inhibition of ALOX15 as a novel, genetically-informed method of treatment of nasal polyposis and related eosinophilic diseases of the airway.

Applicant also identified three additional rare variants with evidence for being protein truncating or damaging to the protein and analyzed them collectively in a gene burden test (Table 4). rs113604586 is a rare (AAF 0.003) missense variant that is predicted to be damaging to the protein by 2 separate functional prediction algorithms. rs550406686 is a rare (AAF 0.0006) stop gain variant in ALOX15 at amino acid position 134 (of 662 for full length protein). rs144038526 is a rare (AAF 0.0003) missense variant that has been experimentally shown to reduce ALOX15 catalytic activity.

TABLE 4
Damaging and Loss of Function Variants in ALOX15

| | | | Functional | Functional |
|---|---|---|---|---|
| Variant | AAF | Coding Consequence | Effect | Evidence |
| rs34210653 | 0.017 | missense (Thr560Met) | LoF | Experimental |
| rs113604586 | 0.003 | missense (Tyr139Cys) | Damaging | Predicted |
| rs550406686 | 0.0006 | stop gain (Glu134Ter) | LoF | Predicted |
| rs144038526 | 0.0003 | missense (Arg402Trp) | Hypomorph | Experimental |

Aggregated variants in the burden test (without T560M) are associated with protection from nasal polyposis and lower blood eosinophils, confirming that these variants are deleterious (Table 5). When aggregated with T560M carriers, there is an increase in statistical significance and a consistent effect size compared to T560M carriers alone. These results are strongly supportive of the hypothesis that inactivation of ALOX15 is protective for eosinophilic airway disease and that therapeutic inhibition will be efficacious for the treatment of these diseases.

TABLE 5
Gene Burden Association Results for ALOX15

| | ALOX15 T560M | | | ALOX15 Burden (w/o T560M) | | | ALOX15 Burden (w/T560M) | | |
|---|---|---|---|---|---|---|---|---|---|
| Phenotype | Effect Size | | P Value | Effect Size | | P Value | Effect Size | | P Value |
| Nasal | 0.38 | (OR) | 1.8E−15 | 0.53 | (OR) | 0.0008 | 0.40 | (OR) | 8.4E−17 |
| Polyposis | | | | | | | | | |
| Eosinophil | −0.02 | (Beta) | 1.5E−65 | −0.016 | (Beta) | 1.4E−09 | −0.019 | (Beta) | 1.3E−72 |
| Count | | | | | | | | | |

Taking a slightly different approach and as a further demonstration of the power of human genetics to model therapeutic target biology, the ALOX15 associations with eosinophilic airway diseases were placed in the context of approved and investigational therapies targeting these diseases, focusing on the novel Th2 cytokine monoclonal antibodies. Variants in or near ALOX15 (rs34210653), IL13 a target of Dupilumab (rs1881457), IL5RA the target of Benralizumab (rs13090169) and TSLP the target of Tezepelumab (rs1898671) were evaluated for association with multiple traits relevant to eosinophilic airways disease. Variants for the Th2 cytokine genes (IL13, IL5RA, and TSLP) were selected for inclusion based on being the variants in or near those genes that were most significantly associated with blood eosinophil counts. Linear regression adjusting for age and sex was carried out, adjusting for population substructure using principal components as covariates. Association results with effect sizes and p-values for association testing with nasal polyps, chronic sinusitis, sinus surgery, nasal steroid use, allergic rhinitis, asthma and blood eosinophil counts are shown in Tables 6A and 6B.

TABLE 6A
Association of ALOX15 and IL13 Variants with Disease Traits

| | ALOX15 rs34210653 | | | IL13 rs1881457 | | |
|---|---|---|---|---|---|---|
| | (EAF = 0.017) | | | (EAF = 0.77) | | |
| Trait | beta | | p-value | beta | | p-value |
| Nasal polyps | 0.38 | (OR) | 1.8E−15 | 0.90 | (OR) | 5.9E−04 |
| Chronic Rhinosinusitis | 0.65 | (OR) | 7.2E−12 | 0.95 | (OR) | 8.8E−03 |
| Sinus Surgery | 0.46 | (OR) | 5.3E−11 | 0.92 | (OR) | 0.01 |
| Nasal Steroid Use | 0.73 | (OR) | 3.5E−05 | 0.95 | (OR) | 0.04 |
| Allergic rhinitis | 0.80 | (OR) | 4.5E−09 | — | | — |
| Asthma | 0.93 | (OR) | 9.1E−06 | 0.91 | (OR) | 4.7E−21 |
| Eosinophil count | −0.02 | (beta) | 1.5E−65 | −0.009 | (beta) | 2.6E−87 |

TABLE 6B
Association of IL5RA and TSLP Variants with Disease Traits

| | IL5RA rs13090169 | | TSLP rs1898671 | | |
|---|---|---|---|---|---|
| | (EAF = 0.89) | | (EAF = 0.68) | | |
| Trait | Effect Size | p-value | Effect Size | | p-value |
| Nasal polyps | — | — | 0.85 | (OR) | 2.4E−12 |
| Chronic Rhinosinusitis | — | — | 0.94 | (OR) | 6.5E−05 |
| Sinus Surgery | — | — | 0.87 | (OR) | 3.2E−08 |
| Nasal Steroid Use | — | — | 0.90 | (OR) | 1.3E−07 |
| Allergic rhinitis | — | — | 0.93 | (OR) | 2.5E−14 |
| Asthma | — | — | 0.91 | (OR) | 6.3E−31 |
| Eosinophil count | −0.004 (beta) | 3.2E−15 | −0.005 | (beta) | 2.0E−52 |

Only results where the p value was <0.05 are shown. There is noted variation in the effect allele frequencies (EAF) and functional consequences of the selected genetic variant. Nonetheless, when comparing patterns of association within each target gene, the evidence is strongly supportive of the comparative efficacy of ALOX15 inhibition. In this analysis, ALOX15 compares extremely favorably to the genes targeted by the new generation of biologics for the treatment of eosinophilic airway diseases, and in the majority of comparisons appears to be much more consistent in its effects on the upper airway.

### Example 2. RNA Synthesis and Duplex Annealing

1. Oligonucleotide Synthesis:

All oligonucleotides are synthesized on an AKTA oligopilot synthesizer or an ABI 394 synthesizer. Commercially available controlled pore glass solid support (dT-CPG, 500A, Prime Synthesis) and RNA phosphoramidites with standard protecting groups, 5′-0-dimethoxytrityl N6-benzoyl-2′-t-butyldimethylsilyl-adenosine-3′-O—N,N′-diisopropyl-2-cyanoethylphosphoramidite, 5′-0-dimethoxytrityl-N4-acetyl-2′-t-butyldimethylsilyl-cytidine-3′-0-N,N′-diisopropyl-2-cyanoethylphosphoramidite, 5′-0-dimethoxytrityl-N2˜isobutryl-2′-t-butyldimethylsilyl-guanosine-3′-O—N,N′-diisopropyl-2-cyanoethylphosphoramidite, and 5′-0-dimethoxytrityl-2′-t-butyldimethylsilyl-uridine-3′-0-N,N′-diisopropyl-2-cyanoethylphosphoramidite (Pierce Nucleic Acids Technologies) are used for the oligonucleotide synthesis unless otherwise specified. The 2′-F phosphoramidites, 5′-O-dimethoxytrityl-N4-acetyl-2′-fluro-cytidine-3′-O— N,N′-diisopropyl-2-cyanoethyl-phosphoramidite and 5′-O-dimethoxytrityl-2′-fluro-uridine-3′-0-N,N′-diisopropyl-2-cyanoethyl-phosphoramidite were purchased from (Promega). All phosphoramidites are used at a concentration of 0.2M in acetonitrile (CH3CN) except for guanosine which is used at 0.2M concentration in 10% THF/ANC (v/v). Coupling/recycling time of 16 minutes is used. The activator is 5-ethyl thiotetrazole (0.75M, American International Chemicals), for the PO-oxidation Iodine/Water/Pyridine is used and the PS-oxidation PADS (2%>) in 2,6-lutidine/ACN (1:1 v/v) is used.

2. Deprotection-1 (Nucleobase Deprotection)

After completion of synthesis, the support is transferred to a 100 ml glass bottle (VWR). The oligonucleotide is cleaved from the support with simultaneous deprotection of base and phosphate groups with 80 mL of a mixture of ethanolic ammonia [ammonia: ethanol (3:1)] for 6.5h at 55° C. The bottle is cooled briefly on ice and then the ethanolic ammonia mixture is filtered into a new 250 ml bottle. The CPG is washed with 2×40 mL portions of ethanol/water (1:1 v/v). The volume of the mixture is then reduced to 30 ml by roto-vap. The mixture is then frozen on dry ice and dried under vacuum on a speed vac.

3. Deprotection-II (Removal of 2′ TBDMS Group)

The dried residue is resuspended in 26 ml of triethylamine, triethylamine trihydro fluoride (TEA.3HF) or pyridine-HF and DMSO (3:4:6) and heated at 60° C. for 90 minutes to remove the tert-butyldimethylsilyl (TBDMS) groups at the 2′ position. The reaction is then quenched with 50 ml of 20 mM sodium acetate and pH adjusted to 6.5, and stored in freezer until purification.

4. Analysis

The oligonucleotides are analyzed by high-performance liquid chromatography (HPLC) prior to purification and selection of buffer and column depends on nature of the sequence and or conjugated ligand.

5. HPLC Purification

The ligand conjugated oligonucleotides are purified reverse phase preparative HPLC. The unconjugated oligonucleotides are purified by anion-exchange HPLC on a TSK gel column packed in house. The buffers are 20 mM sodium phosphate (pH 8.5) in 10% CH3CN (buffer A) and 20 mM sodium phosphate (pH 8.5) in 10% CH3CN, 1M NaBr (buffer B). Fractions containing full-length oligonucleotides are pooled, desalted, and lyophilized. Approximately 0.15 OD of desalted oligonucleotides are diluted in water to 150 μl and then pipetted in special vials for CGE and LC/MS analysis.

Compounds are finally analyzed by LC-ESMS and CGE.

6. siRNA Preparation

For the preparation of siRNA, equimolar amounts of sense and antisense strand are heated in 1×PBS at 95° C. for 5 min and slowly cooled to room temperature.

Integrity of the duplex is confirmed by HPLC analysis.

### Example 3: Design of Antisense Oligonucleotides

Selection of appropriate oligonucleotides is facilitated by using computer programs (e.g. BDT AntiSense Design, IDT OligoAnalyzer) that automatically identify in each given sequence subsequences of 19-25 nucleotides that will form hybrids with a target polynucleotide sequence with a desired melting temperature (usually 50-60° C.) and will not form self-dimers or other complex secondary structures.

Selection of appropriate oligonucleotides is further facilitated by using computer programs that automatically align nucleic acid sequences and indicate regions of identity or homology. Such programs are used to compare nucleic acid sequences obtained, for example, by searching databases such as GenBank or by sequencing PCR products. Comparison of nucleic acid sequences from a range of genes and intergenic regions of a given genome allows the selection of nucleic acid sequences that display an appropriate degree of specificity to the gene of interest. These procedures allow the selection of oligonucleotides that exhibit a high degree of complementarity to target nucleic acid sequences and a lower degree of complementarity to other nucleic acid sequences in a given genome. One skilled in the art will realize that there is considerable latitude in selecting appropriate regions of genes for use in the present embodiments.

The hybridization properties of the oligonucleotides described herein can be determined by one or more in vitro assays as known in the art. For example, the properties of the oligonucleotides described herein can be obtained by determination of binding strength between the target natural antisense and a potential drug molecules using melting curve assay.

The binding strength between the target natural antisense and a potential drug molecule (Molecule) can be estimated using any of the established methods of measuring the strength of intermolecular interactions, for example, a melting curve assay.

Melting curve assay determines the temperature at which a rapid transition from double-stranded to single-stranded conformation occurs for the natural antisense/Molecule complex. This temperature is widely accepted as a reliable measure of the interaction strength between the two molecules.

A melting curve assay is performed using a cDNA copy of the actual natural antisense RNA molecule or a synthetic DNA or RNA nucleotide corresponding to the binding site of the Molecule. Multiple kits containing all necessary reagents to perform this assay are available (e.g. Applied Biosystems Inc. MeltDoctor kit). These kits include a suitable buffer solution containing one of the double strand DNA (dsDNA) binding dyes (such as ABI HRM dyes, SYBR Green, SYTO, etc.). The properties of the dsDNA dyes are such that they emit almost no fluorescence in free form, but are highly fluorescent when bound to dsDNA.

To perform the assay, the cDNA or a corresponding oligonucleotide is mixed with Molecule in concentrations defined by the particular manufacturer's protocols. The mixture is heated to 95° C. to dissociate all preformed dsDNA complexes, then slowly cooled to room temperature or other lower temperature defined by the kit manufacturer to allow the DNA molecules to anneal. The newly formed complexes are then slowly heated to 95° C. with simultaneous continuous collection of data on the amount of fluorescence that is produced by the reaction. The fluorescence intensity is inversely proportional to the amounts of dsDNA present in the reaction. The data can be collected using a real time PCR instrument compatible with the kit (e.g., ABI's StepOne Plus Real Time PCR System or lightTyper instrument, Roche Diagnostics, Lewes, UK).

Melting peaks are constructed by plotting the negative derivative of fluorescence with respect to temperature (−d(Fluorescence)/dT) on the y-axis) against temperature (x-axis) using appropriate software (for example lightTyper (Roche) or SDS Dissociation Curve, ABI). The data is analyzed to identify the temperature of the rapid transition from dsDNA complex to single strand molecules. This temperature is called Tm and is directly proportional to the strength of interaction between the two molecules. Typically, Tm will exceed 40° C.

### Example 4: siRNA-Mediated Knockdown of ALOX15 in A549 Cells

Two siRNAs targeted to the ALOX15 mRNA were demonstrated to downregulate levels of ALOX15 mRNA and 15(S)-HETE (15-hydroxyeicosatetraenoic acid), an arachidonic acid metabolite produced by the ALOX15 gene product 15-lipoxygenase, when administered to cultured A549 cells (a non-small cell lung cancer line).

A549 cells (Sigma Cat. No. 86012804-1VL, Lot No. 16J012) were thawed and maintained according to the manufacturer's protocol in Ham's F12 medium (Corning Cat. No. 10-080-CV, Lot. No. 19717005) supplemented with 10% Fetal Bovine Serum (Atlanta Biologicals, Cat. No. S11550, Lot. No. D17056). The cells were maintained in a T75 flask and subcultured via trypsinization for one week before seeding for experiments.

On Day 0, the A549 cells were seeded at 150,000 cells/mL into a Falcon 24-well tissue culture plate (Cat. No. 353047) at 0.5 mL per well.

On Day 1, cell culture media in appropriate wells was replaced with fresh media containing 20 ng/mL IL-13 (Sino Biologicals, Cat. No. 10369HNAC5, Lot No. LCL11JU0901).

Two siRNAs master mixes were prepared; the ALOX15 siRNA master mix contained 350 uL of Opti-MEM and 3.5 ul of a mixture of the two ALOX15 siRNAs (ThermoFisher Cat. No. 4427037-s1288 Lot No. AS02B02D and s1289 Lot No. AS02B02E, 10 uM stock). The negative control siRNA master mix contained 350 uL of Opti-MEM and 3.5 ul of negative control siRNA (ThermoFisher Cat. No. 4390843, Lot No. AS028XCJ, 10 uM stock) Next, 3 uL of TransIT-X2 (Minis Cat. No. MIR6000, Lot. No. 71113956) was added to each master mix. These were incubated for 15 minutes to allow transfection complexes to form, then 51 ul of the appropriate master mix+TransIT-X2 was added to duplicate wells of A549 cells yielding a final siRNA concentration of 10 nM. The sequence of the ALOX15 siRNA s1288 is as follows: sense strand, 5′-GGAAAUCAUCUAUCGGUAUtt-3′ (SEQ ID NO: 36), antisense strand, 5′-AUACCGAUAGAUGAUUUCCtt-3′ (SEQ ID NO: 37). The two nucleotides at the 3′ end of each sequence are dT. The sequence of the ALOX15 siRNA s1289 is as follows: sense strand, 5′-GCACACUGUUCGAAGCUGAtt-3′ (SEQ ID NO: 38), antisense strand, 5′-UCAGCUUCGAACAGUGUGCtt-3′ (SEQ ID NO: 39). The two nucleotides at the 3′ end of each sequence are dT.

On Day 2, approximately 24 hours post transfection, the wells were supplemented with 10 uM arachidonic acid (Millipore Sigma Cat. No. 10931, Lot No. BCBU4737). On Day 3, approximately 48 hours post transfection, an aliquot of cell supernatant was taken from each well and stored at −80° C. Cells were then lysed using the Cells-to-Ct kit according to the manufacturer's protocol (ThermoFisher Cat. No. 4399002, Lot No. 1707055). In brief, cells were washed with 50 ul using cold 1×PBS and then lysed by adding 49.5 ul of Lysis Solution and 0.5 ul DNase I per well and pipetting up and down 5 times and incubating for 5 minutes at room temperature. The Stop Solution (5 ul/well) was added to each well and mixed by pipetting up and down five times and then incubating at room temperature for 2 minutes. The reverse transcriptase reaction was performed using 22.5 ul of the lysate according to the manufacturer's protocol. Samples were stored at −80° C. until real-time qPCR was performed in triplicate using TaqMan Gene Expression Assays (Applied Biosystems FAM/ALOX15 Cat. No. 4331182-Hs00993765_g1, Lot No. P180613-002-C10; VIC/CyclophilinA Cat. No. 4448489-Hs99999904_ml, Lot No. P180613-003-G09) using a BioRad iCycler.

The cell supernatant was collected 48 hours post siRNA transfection of the A549 cells, and the 15(S)-HETE quantified by ELISA (Cayman Chemical, Cat. No. 534721) according to manufacturer's recommended protocol. Comparison was made between the treatment groups.

The results are shown in FIGS. 10A and 10B. A decrease in ALOX15 mRNA expression in the A549 cells was observed after transfection with the ALOX15 siRNAs (ALOX15_siRNA) compared to ALOX15 mRNA levels in A549 cells transfected with the non-specific control siRNA (con_siRNA) 48 hours after transfection (p=0.04) (FIG. 10A). A decrease in the amount of 15(S)-HETE in the media from wells containing A549 cells transfected with the ALOX15 siRNAs (ALOX15_siRNA) relative to the amount of 15(S)-HETE in the media from wells containing A549 cells transfected with a non-specific control siRNA (con_siRNA) 48 hours after transfection (p=0.03) was observed (FIG. 10B). These results show that the ALOX15 siRNAs elicited knockdown of ALOX15 mRNA in A549 cells and that the decrease in ALOX15 expression correlated with a decrease in 15(S)-HETE production. These results confirm that siRNA knockdown of ALOX15 mRNA recapitulates the functional effects of the ALOX15 rs34210653 missense variant (T560M) described above. Specifically, both siRNA knockdown of ALOX15 mRNA and a threonine to methionine exchange at position 560 of the ALOX15 protein result in reduced ALOX15-mediated production of 15(S)-HETE.

### Example 5: siRNA Inhibition of 15-Lipoxygenase in a Murine Model for Nasal Polyposis

In this experiment, a murine model of nasal polyps is used to evaluate the effect of siRNA inhibition of ALOX15. The murine model consists of inducing chronic rhinitis with ovalbumin (OVA) treatment, followed by a combination of ovalbumin and Staphylococcus aureus enterotoxin B (SEB) treatment leading to nasal polyps formation. This model has characteristics of the human disease.

2-week-old female BALB/c mice are obtained from Charles River Laboratories. The animals are kept in environmentally controlled rooms under specific pathogen-free conditions (temperature, 20-26° C.); humidity, 30-70%) with a 12-hour light-dark cycle for 2 weeks before use. Food and water are available ad libitum. All animals are used in accordance with animal care guidelines.

Briefly, mice are divided into three groups. Group A (n=5) is a control group in which mice are not treated with reagent. Group B (n=5) is a nasal polyp+scrambled siRNA group (NP+siRNA scrambled) and Group C (n=5) is a nasal polyp group+ALOX15 siRNA group (NP+siRNA ALOX15). For groups B and C mice are sensitized with an intraperitoneal injection of 25 ug of OVA plus 2 mg of aluminum hydroxide on days 0 and 7. From day 14 to day 20 mice are nasally challenged daily with 6% OVA. From day 20, 6% OVA+Staphylococcus aureus enterotoxin B (10 ng) is instilled into the nasal cavity of mice three times per week for 8 weeks.

At weeks 0 and 4 of OVA+SEB, siRNA is applied. Anesthesia for administration of siRNA is achieved with intraperitoneal injection of 0.2 ml nembutal (5 mg/ml). The ALOX15 targeting siRNA is cross-reactive with the mouse and human ALOX15 mRNA and consists of a double stranded RNA oligonucleotide with sense and antisense sequences as follows: 5′ mGsmCmUmGfUmGmCmUfGmAmAmGfAmAmGmUfUfCmAsdTsdT 3′ (SEQ ID NO: 14) and 5′ mUsfGmAmAmCfUfUmCmUfUfCfAmGfCmAmCmAfGmCsdTsdT 3′ (SEQ ID NO: 15). This siRNA targets nucleotide positions 1890-1908 of the murine ALOX15 mRNA (GenBank Acc. #NM_009660.3) and positions 1883-2001 of the human ALOX15 mRNA (GenBank Acc. #NM_001140.4). The non-targeting control siRNA consists of the following sense and antisense sequences: 5′ mGsmUmCmCfAmUmCmAmfGmCmUmCmfGmGmUmUfAfGmAsdTsdT 3′ (SEQ ID NO: 22) and 5′ mUsfCmUmAmAfCfCmGmAfGfCfUmGfAmUmGmGfAmCdTdT 3′ (SEQ ID NO: 23). The letter “m” before the nucleotide indicates a 2′O-methyl substitution, the letter “f” indicates a 2′fluoro substitution, the letter “d” indicates a deoxyribonucleotide substitution, and the letter “s” indicates a phosphorothioate linkage. Naked siRNA is resuspended in Opti-MEM, and 10 ug siRNA in 5 ul volume is delivered to each nostril with a micropipette.

At the end of eight weeks of OVA+SEB and siRNA treatment, mice are killed by cervical dislocation following injection of 0.3 ml nembutal. Nasal cavity samples are prepared using a large scalpel to remove the snout with a transverse cut behind the back molars. The external nares are flushed with PBS to wash out any blood within the nasal cavity. Histologic quantification of the number of nasal polyps and the amount of eosinophil infiltration is performed using hematoxylin and eosin (H&E) staining of the nasal cavity. The number of polyps and amount of eosinophilic infiltration is compared between the NP+siRNA scrambled group and the NP+siRNA ALOX15 group. Additionally, nasal mucosa is removed using a small curette after bisecting the nasal tissue sagitally along the nasal septum. Cell lysates are prepared using a RIPA buffer, and these lysates are used to measure arachidonic acid metabolites of ALOX15 including prostaglandin E2, cysteinyl leukotrienes (LTC4, D4, and E4), and 15(S)-HETE. The metabolites are quantified by ELISA (Cayman Chemical) according to manufacturer's recommended protocol. Metabolite levels are compared between the NP+siRNA scrambled group and the NP+siRNA ALOX15 group.

In another experiment, the ALOX15 targeting siRNA consists of a double stranded RNA oligonucleotide with sense and antisense sequences as follows: 5′ GGAGUACACGUUCCCCUGUUAdTdT 3′ (SEQ ID NO: 6) and 5′ UAACAGGGGAACGUGUACUCCdTdT 3′ (SEQ ID NO: 7). This siRNA targets nucleotide positions 273-293 of the murine ALOX15 reading frame. The scrambled (control) siRNA consists of the following sense and antisense sequences: 5′ GUCCAUCAGCUCGGUUAGACUdTdT 3′ (SEQ ID NO: 8) and 5′ AGUCUAACCGAGCUGAUGGACdTdT 3′ (SEQ ID NO: 9). The letter d in front of a nucleotide indicates it is a deoxyribonucleotide.

### Example 6. Antisense Oligonucleotide Inhibition of 15-lipoxygenase in a Murine Model for Nasal Polyposis

In this experiment, a murine model of nasal polyps is used to evaluate the effect of antisense inhibition of ALOX15 polynucleotides including mRNA. The murine model consists of inducing chronic rhinitis with ovalbumin (OVA) treatment, followed by a combination of ovalbumin and Staphylococcus aureus enterotoxin B (SEB) treatment leading to nasal polyps formation. This model has characteristics of the human disease.

2-week-old female BALB/c mice are obtained from Charles River Laboratories. The animals are kept in environmentally controlled rooms under specific pathogen-free conditions (temperature, 20-26° C.); humidity, 30-70%) with a 12-hour light-dark cycle for 2 weeks before use. Food and water are available ad libitum. All animals are used in accordance with animal care guidelines.

Briefly, mice are divided into three groups. Group A (n=5) is a control group in which mice are not treated with reagent. Group B (n=5) is a nasal polyp+control antisense oligonucleotide group (NP+antisense control) and Group C (n=5) is a nasal polyp group+ALOX15 antisense group (NP+antisense ALOX15).). For groups B and C, mice are sensitized with an intraperitoneal injection of 25 ug of OVA plus 2 mg of aluminum hydroxide on days 0 and 7. From day 14 to day 20 mice are nasally challenged daily with 6% OVA. From day 20, 6% OVA+Staphylococcus aureus enterotoxin B (10 ng) are instilled into the nasal cavity of mice three times per week for 8 weeks.

At weeks 0 and 4 of OVA+SEB, the antisense oligonucleotide is applied. Anesthesia for administration of antisense oligonucleotide is achieved with intraperitoneal injection of 0.2 ml nembutal (5 mg/ml). The ALOX15 targeting antisense oligonucleotide is cross-reactive with the mouse and human ALOX15 mRNA and consists of an oligonucleotide with sequence as follows: 5′ mCsmTsmGsmAsmAsdCsdTsdTsdCsdTsdTsdCsdAsdGsdCsmAsmCsmAsmGsmC 3′ (SEQ ID NO: 24).

This antisense oligonucleotide targets nucleotide positions 1890-1909 of the murine ALOX15 mRNA (GenBank Acc. #NM_009660.3) and positions 1883-2002 of the human ALOX15 mRNA (GenBank Acc. #NM_001140.4). The non-targeting control oligonucleotide consists of the following sense and antisense sequences: 5′ mTsmCsmTsmAsmAsdCsdCsdGsdAsdGsdCsdTsdGsdAsdTsmGsmGsmAsmCsmT 3′ (SEQ ID NO: 25). The letter “m” before the nucleotide indicates a 2′O-methoxyethyl substitution, the letter “d” indicates a deoxyribonucleotide substitution, and the letter “s” indicates a phosphorothioate linkage. Naked antisense oligonucleotide is resuspended in Opti-MEM, and 100 ug siRNA in 5 ul volume is delivered to each nostril with a micropipette.

At the end of eight weeks of OVA+SEB and antisense oligonucleotide treatment, mice are killed by cervical dislocation following injection of 0.3 ml nembutal. Nasal cavity samples are prepared using a large scalpel to remove the snout with a transverse cut behind the back molars. The external nares are flushed with PBS to wash out any blood within the nasal cavity. Histologic quantification of the number of nasal polyps and the amount of eosinophil infiltration is performed using hematoxylin and eosin (H&E) staining of the nasal cavity. The number of polyps and amount of eosinophilic infiltration is compared between the NP+antisense control group and the NP+antisense ALOX15 group. Additionally, nasal mucosa is removed using a small curette after bisecting the nasal tissue sagitally along the nasal septum. Cell lysates are prepared using a RIPA buffer, and these lysates are used to measure arachidonic acid metabolites of ALOX15, including prostaglandin E2, cysteinyl leukotrienes (LTC4, D4, and E4), and 15(S)-HETE. The metabolites are quantified by ELISA (Cayman Chemical) according to manufacturer's recommended protocol. Metabolite levels are compared between the NP+antisense control group and the NP+antisense ALOX15 group.

### Example 7. Small Molecule Inhibition of 15-lipoxygenase in a Murine Model for Nasal Polyposis

In this experiment, a murine model of nasal polyposis is used to evaluate the effect of inhibition of 15-lipoxygenase. The murine model consists of inducing chronic rhinitis with ovalbumin (OVA) treatment, followed by a combination of ovalbumin and Staphylococcus aureus enterotoxin B (SEB) treatment leading to nasal polyp formation. This model has characteristics of the human disease.

2-week-old female BALB/c mice are obtained from Charles River Laboratories. The animals are kept in environmentally controlled rooms under specific pathogen-free conditions (temperature, 20-26° C.); humidity, 30-70%) with a 12-hour light-dark cycle for 2 weeks before use. Food and water are available ad libitum. All animals are used in accordance with animal care guidelines.

Nasal polyposis is induced in age-matched (4-week-old) mice divided into five groups. Group A (n=5) is a control group in which mice are not treated with reagent. For groups B-E (n=5 for each), mice are sensitized with an intraperitoneal injection of 25 ug of OVA plus 2 mg of aluminum hydroxide on days 0 and 7. From Day 14 to Day 20 mice are nasally challenged daily with 6% OVA. From Day 20, 6% OVA+Staphylococcus aureus enterotoxin B (10 ng) is instilled into the nasal cavity of mice three times per week for 8 weeks.

From week 4, animals in groups B-D receive ALOX15 inhibitor and animals in group E receive vehicle only (1 part DMSO, 9 parts PBS) three times a week for 4 weeks. Anesthesia during intranasal administration is achieved with intraperitoneal injection of 0.2 ml nembutal (5 mg/ml).

At the end of eight weeks of OVA+SEB and inhibitor treatment, mice are euthanized by cervical dislocation following injection of 0.3 ml nembutal. Nasal cavity samples are prepared using a large scalpel to remove the snout with a transverse cut behind the back molars. The external nares are flushed with PBS to wash out any blood within the nasal cavity. Histologic quantification of the number of nasal polyps and the amount of eosinophil infiltration is performed using hematoxylin and eosin (H&E) staining of the nasal cavity. The number of polyps and amount of eosinophilic infiltration is compared between all groups. Additionally, nasal mucosa is removed using a small curette after bisecting the nasal tissue sagitally along the nasal septum. Cell lysates are prepared using a RIPA buffer, and these lysates are used to measure arachidonic acid metabolites of 15-lipoxygenase including prostaglandin E2, cysteinyl leukotrienes (LTC4, D4, and E4), and 15(S)-HETE. The metabolites are quantified by ELISA (Cayman Chemical) according to manufacturer's recommended protocol. Comparisons are made between the control group, groups treated with the test compounds and the group treated with vehicle only.

### Example 8: siRNA Inhibition of 15-lipoxygenase in in Human Cells

In this experiment siRNA inhibition of ALOX15 is performed in primary human nasal epithelial cells (pHNEC) and a human eosinophil cell line (EoL-1), to evaluate the effect on arachidonic acid metabolite production under conditions of no treatment versus IL13 treatment. IL13 treatment serves to induce ALOX15 expression, and is a critical regulator of the Th2 inflammation present in human nasal polyposis patients.

EoL-1 cells are initially cultured in the presence of dbcAMP (100 uM) for 7 days to differentiate to a mature eosinophil cell type. Next, differentiated EoL-1 cells and pHNECs are transfected in a 24-well plate with a scrambled or ALOX15 targeting siRNA immediately followed by +/−supplementation with IL13 (20 ng/ml). Additionally, all wells are supplemented with 10 uM arachidonic acid so that substrate is a not a limiting factor in metabolite production. The ALOX15 targeting siRNA consists of a double stranded RNA oligonucleotide with sense and antisense sequences as follows: 5′ GCUGUGCUGAAGAAGUUCAdTdT 3′ (SEQ ID NO: 16) and 5′ UGAACUUCUUCAGCACAGCdTdT 3′ (SEQ ID NO: 17). This siRNA targets nucleotide positions 1883-2001 of the human ALOX15 mRNA (GenBank Acc. #NM_001140.4). The non-targeting control siRNA consists of the following sense and antisense sequences: 5′ GUCCAUCAGCUCGGUUAGAdTdT 3′ (SEQ ID NO: 18) and 5′ UCUAACCGAGCUGAUGGACdTdT 3′ (SEQ ID NO: 19). The letter “d” indicates a deoxyribonucleotide substitution. Briefly, transfections are performed using TransIT TKO (Mirus) following the manufacturer's recommended protocol. For each well, 1.4 ul siRNA (10 uM stock), 2.5 ul TransIT-TKO, and 50 ul OptiMEM are mixed, incubated at room temperature for 30 minutes, and added dropwise to each well.

At 72 hrs post-transfection, supernatant is collected, cells are trypsinized, and cell lysates prepared using a RIPA buffer. Supernatants are used to measure arachidonic acid metabolites of ALOX15 including prostaglandin E2, cysteinyl leukotrienes (LTC4, D4, and E4), and 15(S)-HETE. The metabolites are quantified by ELISA (Cayman Chemical) according to manufacturer's recommended protocol, and a comparison is made between the various treatment groups. Cell lysates are used to perform Western blots, using an ALOX15 antibody (Abcam) and a GAPDH antibody (Abcam) as a loading control.

In another experiment, the ALOX15 targeting siRNA consists of a double stranded RNA oligonucleotide with sense and antisense sequences as follows: 5′ GGUGGAAGUACCGGAGUAUCUdTdT 3′ (SEQ ID NO: 10) and 5′ AGAUACUCCGGUACUUCCACCdTdT 3′ (SEQ ID NO: 11). This siRNA targets nucleotide positions 150-170 of the human ALOX15 reading frame. The scrambled (control) siRNA consists of the following sense and antisense sequences: 5′ GUUGUACAGCAUGCGGAGAGUdTdT 3′ (SEQ ID NO: 12) and 5′ ACUCUCCGCAUGCUGUACAACdTdT 3′ (SEQ ID NO: 13). The letter d in front of a nucleotide indicates it is a deoxyribonucleotide.

### Example 9. Antisense Oligonucleotide Inhibition of 15-Lipoxygenase in Human Cells

In this experiment antisense oligonucleotide inhibition of ALOX15 is performed in primary human nasal epithelial cells (pHNEC) and a human eosinophil cell line (EoL-1), to evaluate the effect on arachidonic acid metabolite production under conditions of no treatment versus IL13 treatment. IL13 treatment serves to induce ALOX15 expression, and is a critical regulator of the Th2 inflammation present in human nasal polyposis patients.

EoL-1 cells are initially cultured in the presence of dbcAMP (100 uM) for 7 days to differentiate to a mature eosinophil cell type. Next, differentiated EoL-1 cells and pHNECs are transfected in a 24-well plate with a scrambled or ALOX15 targeting antisense oligonucleotide immediately followed by +/−supplementation with IL13 (20 ng/ml). Additionally, all wells are supplemented with 10 uM arachidonic acid so that substrate is a not a limiting factor in metabolite production. The ALOX15 targeting antisense oligonucleotide consists of sequences as follows: 5′ mCsmTsmGsmAsmAsdCsdTsdTsdCsdTsdTsdCsdAsdGsdCsmAsmCsmAsmGsmC 3′ (SEQ ID NO: 24). This antisense oligonucleotide targets nucleotide positions 1883-2002 of the human ALOX15 mRNA (GenBank Acc. #NM_001140.4). The non-targeting control antisense oligonucleotide consists of the following sequence: 5′ mTsmCsmTsmAsmAsdCsdCsdGsdAsdGsdCsdTsdGsdAsdTsmGsmGsmAsmCsmT 3′ (SEQ ID NO: 25). The letter “m” before the nucleotide indicates a 2′O-methoxyethyl substitution, the letter “d” indicates a deoxyribonucleotide substitution, and the letter “s” indicates a phosphorothioate linkage. Briefly, transfections are performed using TransIT TKO (Mirus) following the manufacturer's recommended protocol. For each well, 1.4 ul antisense oligonucleotide (1 mM stock), 2.5 ul TransIT-TKO, and 50 ul OptiMEM are mixed, incubated at room temperature for 30 minutes, and added dropwise to each well.

At 72 hrs post-transfection, supernatant is collected, cells are trypsinized, and cell lysates prepared using a RIPA buffer. Supernatants are used to measure arachidonic acid metabolites of ALOX15 including prostaglandin E2, cysteinyl leukotrienes (LTC4, D4, and E4), and 15(S)-HETE. The metabolites are quantified by ELISA (Cayman Chemical) according to manufacturer's recommended protocol, and a comparison is made between the various treatment groups. Cell lysates are used to perform Western blots, using an ALOX15 antibody (Abcam) and a GAPDH antibody (Abcam) as a loading control.

### Example 10. Small Molecule Inhibition of 15-Lipoxygenase in Human Cells

In this experiment inhibition of ALOX15 is performed in primary human nasal epithelial cells (pHNEC) and a human eosinophil cell line (EoL-1), to evaluate the effect on arachidonic acid metabolite production under conditions of no treatment versus IL13 treatment. IL13 treatment serves to induce ALOX15 expression, and is a critical regulator of the Th2 inflammation present in human nasal polyposis patients.

EoL-1 cells are initially cultured in the presence of dbcAMP (100 uM) for 7 days to differentiate to a mature eosinophil cell type. Next, differentiated EoL-1 cells and pHNECs are treated in a 24-well plate with ALOX15 inhibitor immediately followed by +/−supplementation with IL13 (20 ng/ml). Additionally, all wells are supplemented with 10 uM arachidonic acid so that substrate is a not a limiting factor in metabolite production.

At 72 hrs post-treatment, supernatant is collected, cells are trypsinized, and cell lysates prepared using a RIPA buffer. Supernatants are used to measure arachidonic acid metabolites of ALOX15 including prostaglandin E2, cysteinyl leukotrienes (LTC4, D4, and E4), and 15(S)-HETE. The metabolites are quantified by ELISA (Cayman Chemical) according to manufacturer's recommended protocol, and comparison made between the various treatment groups. Cell lysates are used to perform Western blots, using an ALOX15 antibody (Abeam) and a GAPDH antibody (Abeam) as a loading control.

### Example 11: siRNA Inhibition of 15-Lipoxygenase in a Rabbit Model for Nasal Polyposis

In this experiment, a rabbit model of nasal polyps is used to evaluate the effect of siRNA inhibition of ALOX15. The rabbit model consists of inducing eosinophilic nasal polyps by eliciting an allergic reaction in animals with ovalbumin (OVA) and poly-L-arginine treatment. This model has characteristics of the human disease.

Male New Zealand white rabbits are obtained from Charles River Laboratories. The animals are kept in environmentally controlled rooms under specific pathogen-free conditions (temperature, 20-26° C.); humidity, 30-70%) with a 12-hour light-dark cycle for 2 weeks before use. Food and water are available ad libitum. All animals are used in accordance with animal care guidelines.

Maxillary sinusitis is induced in age-matched (13-week-old) rabbits divided into four groups. Group A (n=4) is a control group in which rabbits are treated with neither reagent nor surgery. For groups B-D (n=6 for each), rabbits are sensitized by subcutaneous injection with 1 mL of saline containing 2.5% OVA plus 0.4% alum on days 0 and 7. On day 14, under anesthesia with i.v. injection of 25 mg/kg of pentobarbital sodium (Nembutal, Dainippon Sumitomo Pharma, Osaka, Japan), nasal dorsa are incised to expose maxillary sinus cavities and both sides of the ostia are occluded with plugs of sterile cotton wool and butylcyanoacrylate tissue glue (Histoacryl; B. Braun, Melsungen AG, Germany) under a microscope. After 2 weeks of wound closure, OVA is administered into both sides of the maxillary sinuses (0.5 mL/sinus of 2.5% OVA in saline, three times a week for 2 weeks). Thereafter, the animals in groups B-D receive 5 mg/mL poly-L-arginine in saline three times a week for 4 weeks.

After induction of maxillary sinusitis with OVA and poly-L-arginine, the animals receive 0.5 mL/sinus of saline (group B), 2 mg/ml ALOX15 siRNA in saline (group C), or 2 mg/mL control siRNA in saline (group D) three times a week for 4 weeks. One week after the last administration into the maxillary sinus, the rabbits are killed by i.v. injection of pentobarbital sodium and the anterior nasal region with the attached bone, excluding the ocular bulb, is dissected. The mucosal tissues for gene analysis are collected from the right side of the maxillary sinus and stored in RNAlater RNA stabilization reagent (Ambion, Austin, Tex.) at 4° C. until analysis. The left side of the maxillary sinus is used for histopathological analysis. Anesthesia for administration of siRNA is achieved with intraperitoneal injection of 0.2 ml nembutal (5 mg/ml).

The ALOX15 targeting siRNA is cross-reactive with the rabbit and human ALOX15 mRNA and consists of a double stranded RNA oligonucleotide with sense and antisense sequences as follows: 5′ mCsmUmGmUfGmGmAmUfGmAmGmCfGmAmUmUfUfCmUsdTsdT 3′ (SEQ ID NO: 20) and 5′ mAsfGmAmAmAfUfCmGmCfUfCfAmUfCmCmAmCfAmGsdTsdT 3′ (SEQ ID NO: 21). This siRNA targets nucleotide positions 512-530 of the rabbit ALOX15 mRNA (GenBank Acc. #NM_009660.3) and positions 504-522 of the human ALOX15 mRNA (GenBank Acc. #NM_001140.4). The non-targeting control siRNA consists of the following sense and antisense sequences: 5′ mGsmUmCmCfAmUmCmAmfGmCmUmCmfGmGmUmUfAfGmAsdTsdT (SEQ ID NO: 22) 3′ and 5′ mUsfCmUmAmAfCfCmGmAfGfCfUmGfAmUmGmGfAmCdTdT (SEQ ID NO: 23) 3′. The letter “m” before the nucleotide indicates a 2′O-methyl substitution, the letter “f” indicates a 2′fluoro substitution, the letter “d” indicates a deoxyribonucleotide substitution, and the letter “s” indicates a phosphorothioate linkage. Naked siRNA is resuspended in Opti-MEM, and 400 ug siRNA in 200 ul volume is delivered to each nostril with a micropipette.

Antibodies to OVA are measured using enzyme-linked immunosorbent assay (ELISA). Blood samples are collected from the pinna vein on day 13 to measure OVA-specific IgG levels. ELISA is performed according to the protocol of a previous study. The titers of samples are calculated by comparison with internal standard serum, prepared from the rabbits immunized with 2.5% OVA plus 0.4% alum eight times. The value of this standard is arbitrarily calculated as 10,000 U/ml.

Histopathological analysis of nasal tissue is accomplished on tissue fixed with 10% neutral buffered formalin solution for 1 week, decalcified in 0.5 mol/L of ethylenediaminetetraacetic acid at 37° C., embedded in paraffin, and cut into 2-μm-thick sections. After hematoxylin-eosin staining, histopathological analysis is performed by selecting a representative field of mucosa per sinus where the most prominent change is detected with 200× magnification. The number of eosinophils and the area of mucosa in each field are measured to calculate the density of eosinophils (cells/mm2). The width of the lamina propria (μm) is measured as an indirect indication of mucosal hypertrophy. The degree of polyp formation is graded semi-quantitatively according to the following score: 0=little or no polyp formation detected; 1=slight (slight prominence of mucosa); 2=moderate (polyp of a size from one-quarter to one-half of the field); 3=severe (polyp of a size more than one-half of the field).

ALOX15 mRNA knockdown is quantified using real-time polymerase chain reaction. Total RNA is reverse transcribed to cDNA using a First-Strand III cDNA Synthesis kit (Invitrogen, Carlsbad, Calif.). Then, real-time quantitative polymerase chain reaction (PCR) is performed using the ABI Prism 7900 Sequence Detection System (Applied Biosystems, Foster City, Calif.). Amplification by PCR is performed according to the manufacturer's protocols (Applied Biosystems). Primers and probes for ALOX15 for use in this study and β-actin are designed with the assistance of the computer program Primer Express (Applied Biosystems). Searches using a nucleotide basic local alignment search tool (BLASTN) database are conducted to confirm their specificity and the absence of DNA polymorphisms.

### Example 12. Antisense Oligonucleotide Inhibition of 15-Lipoxygenase in a Rabbit Model for Nasal Polyposis

In this experiment, a rabbit model of nasal polyps is used to evaluate the effect of antisense oligonucleotide inhibition of ALOX15. The rabbit model consists of inducing eosinophilic nasal polyps by eliciting an allergic reaction in animals with ovalbumin (OVA) and poly-L-arginine treatment. This model has characteristics of the human disease.

Male New Zealand white rabbits are obtained from Charles River Laboratories. The animals are kept in environmentally controlled rooms under specific pathogen-free conditions (temperature, 20-26° C.); humidity, 30-70%) with a 12-hour light-dark cycle for 2 weeks before use. Food and water are available ad libitum. All animals are used in accordance with animal care guidelines.

Maxillary sinusitis is induced in age-matched (13-week-old) rabbits divided into four groups. Group A (n=4) is a control group in which rabbits are treated with neither reagent nor surgery. For groups B-D (n=6 for each), rabbits are sensitized by subcutaneous injection with 1 mL of saline containing 2.5% OVA plus 0.4% alum on days 0 and 7. On day 14, under anesthesia with i.v. injection of 25 mg/kg of pentobarbital sodium (Nembutal, Dainippon Sumitomo Pharma, Osaka, Japan), nasal dorsa are incised to expose maxillary sinus cavities and both sides of the ostia are occluded with plugs of sterile cotton wool and butylcyanoacrylate tissue glue (Histoacryl; B. Braun, Melsungen AG, Germany) under a microscope. After 2 weeks of wound closure, OVA is administered into both sides of the maxillary sinuses (0.5 mL/sinus of 2.5% OVA in saline, three times a week for 2 weeks). Thereafter, the animals in groups B-D receive 5 mg/mL poly-L-arginine in saline three times a week for 4 weeks.

After induction of maxillary sinusitis with OVA and poly-L-arginine, the animals receive 0.5 mL/sinus of saline (group B), 20 mg/ml ALOX15 antisense oligonucleotide in saline (group C), or 20 mg/mL control antisense oligonucleotide in saline (group D) three times a week for 4 weeks. One week after the last administration into the maxillary sinus, the rabbits are killed by i.v. injection of pentobarbital sodium and the anterior nasal region with the attached bone, excluding the ocular bulb, is dissected. The mucosal tissues for gene analysis are collected from the right side of the maxillary sinus and stored in RNAlater RNA stabilization reagent (Ambion, Austin, Tex.) at 4° C. until analysis. The left side of the maxillary sinus is used for histopathological analysis. Anesthesia for administration of the antisense oligonucleotides is achieved with intraperitoneal injection of 0.2 ml nembutal (5 mg/ml). The ALOX15 targeting antisense oligonucleotide is cross-reactive with the rabbit and human ALOX15 mRNA and consists of an oligonucleotide with sequence as follows: 5′ mCsmAsmGsmAsmAsdAsdTsdCsdGsdCsdTsdCsdAsdTsdCsmCsmAsmCsmAsmG 3′ (SEQ ID NO:5). This antisense oligonucleotide targets nucleotide positions 512-531 of the rabbit ALOX15 mRNA (GenBank Acc. #NM_009660.3) and positions 504-523 of the human ALOX15 mRNA (GenBank Acc. #NM_001140.4). The non-targeting control antisense oligonucleotide consists of the following sequence: 5′ mTsmCsmTsmAsmAsdCsdCsdGsdAsdGsdCsdTsdGsdAsdTsmGsmGsmAsmCsmT 3′ (SEQ ID NO: 25). The letter “m” before the nucleotide indicates a 2′O-methoxyethyl substitution, the letter “d” indicates a deoxyribonucleotide substitution, and the letter “s” indicates a phosphorothioate linkage. Naked antisense oligonucleotide is resuspended in Opti-MEM, and 400 ug siRNA in 200 ul volume is delivered to each nostril with a micropipette.

Antibodies to OVA are measured using enzyme-linked immunosorbent assay (ELISA). Blood samples are collected from the pinna vein on day 13 to measure OVA-specific IgG levels. ELISA is performed according to the protocol of a previous study. The titers of samples are calculated by comparison with internal standard serum, prepared from the rabbits immunized with 2.5% OVA plus 0.4% alum eight times. The value of this standard is arbitrarily calculated as 10,000 U/ml.

Histopathological analysis of nasal tissue is accomplished on tissue fixed with 10% neutral buffered formalin solution for 1 week, decalcified in 0.5 mol/L of ethylenediaminetetraacetic acid at 37° C., embedded in paraffin, and cut into 2-μm-thick sections. After hematoxylin-eosin staining, histopathological analysis is performed by selecting a representative field of mucosa per sinus where the most prominent change is detected with 200× magnification. The number of eosinophils and the area of mucosa in each field are measured to calculate the density of eosinophils (cells/mm2). The width of the lamina propria (μm) is measured as an indirect indication of mucosal hypertrophy. The degree of polyp formation is graded semiquantitatively according to the following score: 0=little or no polyp formation detected; 1=slight (slight prominence of mucosa); 2=moderate (polyp of a size from one-quarter to one-half of the field); 3=severe (polyp of a size more than one-half of the field).

ALOX15 mRNA is quantified using real-time polymerase chain reaction. Total RNA is reverse transcribed to cDNA using a First-Strand III cDNA Synthesis kit (Invitrogen, Carlsbad, Calif.). Then, real-time quantitative polymerase chain reaction (PCR) is performed using the ABI Prism 7900 Sequence Detection System (Applied Biosystems, Foster City, Calif.). Amplification by PCR is performed according to the manufacturer's protocols (Applied Biosystems). Primers and probes for ALOX15 for use in this study and (3-actin are designed with the assistance of the computer program Primer Express (Applied Biosystems). Searches using a nucleotide basic local alignment search tool (BLASTN) database are conducted to confirm their specificity and the absence of DNA polymorphisms.

### Example 13: Small Molecule Inhibition of 15-Lipoxygenase in a Rabbit Model for Nasal Polyposis

In this experiment, a rabbit model of nasal polyposis is used to evaluate the effect of inhibition of 15-lipoxygenase. In the rabbit model eosinophilic nasal polyps are induced by eliciting an allergic reaction in animals with ovalbumin (OVA) and poly-L-arginine treatment. This model has characteristics of the human disease.

Male New Zealand white rabbits are obtained from Charles River Laboratories. The animals are kept in environmentally controlled rooms under specific pathogen-free conditions (temperature, 20-26° C.); humidity, 30-70%) with a 12-hour light-dark cycle for 2 weeks before use. Food and water are available ad libitum. All animals are used in accordance with animal care guidelines.

Maxillary sinusitis is induced in age-matched (13-week-old) rabbits divided into five groups. Group A (n=4) is a control group in which rabbits are treated with neither reagent nor surgery. For groups B-E (n=6 for each), rabbits are sensitized by subcutaneous injection with 1 mL of saline containing 2.5% OVA plus 0.4% alum on days 0 and 7. On day 14, under anesthesia with i.v. injection of 25 mg/kg of pentobarbital sodium (Nembutal, Dainippon Sumitomo Pharma, Osaka, Japan), nasal dorsa are incised to expose maxillary sinus cavities and both sides of the ostia are reoccluded with plugs of sterile cotton wool and butylcyanoacrylate tissue glue (Histoacryl; B. Braun, Melsungen AG, Germany) under a microscope. After 2 weeks of wound closure, OVA is administered into both sides of the maxillary sinuses (0.5 mL/sinus of 2.5% OVA in saline, three times a week for 2 weeks). Thereafter, the animals in groups B-D receive 5 mg/mL poly-L-arginine in saline three times a week for 4 weeks.

After induction of maxillary sinusitis with OVA and poly-L-arginine, the animals in groups B-D receive ALOX15 inhibitor and animals in group E receive vehicle only (1 part DMSO, 9 parts PBS) three times a week for 4 weeks. One week after the last administration into the maxillary sinus, the rabbits are sacrificed by i.v. injection of pentobarbital sodium and the anterior nasal region with the attached bone, excluding the ocular bulb, is dissected. The mucosal tissues for gene analysis are collected from the right side of the maxillary sinus and stored in RNAlater RNA stabilization reagent (Ambion, Austin, Tex.) at 4° C. until analysis. The left side of the maxillary sinus is used for histopathological analysis. Anesthesia for administration of the test compounds is achieved with intraperitoneal injection of 0.2 ml nembutal (5 mg/ml).

Antibodies to OVA are measured using enzyme-linked immunosorbent assay (ELISA). Blood samples are collected from the pinna vein on day 13 to measure OVA-specific IgG levels. ELISA is performed according to the protocol of a previous study. The titers of samples are calculated by comparison with internal standard serum, prepared from the rabbits immunized with 2.5% OVA plus 0.4% alum eight times. The value of this standard is arbitrarily calculated as 10,000 U/ml.

Histopathological analysis of nasal tissue is carried out on tissue fixed with 10% neutral buffered formalin solution for 1 week, decalcified in 0.5 mol/L of ethylenediaminetetraacetic acid at 37° C., embedded in paraffin, and cut into 2-μm-thick sections. After hematoxylin-eosin staining, histopathological analysis is performed by selecting a representative field of mucosa per sinus where the most prominent change is detected with 200× magnification. The number of eosinophils and the area of mucosa in each field are measured to calculate the density of eosinophils (cells/mm2). The width of the lamina propria (μm) is measured as an indirect indication of mucosal hypertrophy. The degree of polyp formation is graded semiquantitatively according to the following score: 0=little or no polyp formation detected; 1=slight (slight prominence of mucosa); 2=moderate (polyp of a size from one-quarter to one-half of the field); 3=severe (polyp of a size more than one-half of the field).

The foregoing is considered as illustrative only of the principles of the disclosure. Furthermore, since numerous modifications and changes will readily occur to those skilled in the art, it is not desired to limit the embodiments of this disclosure to the exact construction and operation shown and described. Therefore, all suitable modifications and equivalents fall within the scope of this disclosure.

What is claimed is:

1. A method of treating one or more disorders of the upper and lower airway in a subject in need thereof comprising administering to the subject an inhibitor of arachidonate 15-lipoxygenase (ALOX15) wherein the one or more disorders of the upper and lower airway comprises nasal polyposis, chronic sinusitis, allergic rhinitis, asthma, or NSAID-exacerbated respiratory disease.

2. The method of claim 1, wherein the inhibitor of ALOX15 is delivered systemically to the subject.

3. The method of claim 1, wherein the inhibitor of ALOX15 is delivered locally to the subject.

4. The method of claim 1, wherein the inhibitor of ALOX15 is delivered locally to the nasal epithelium of the subject.

5. The method of claim 1, wherein the one or more disorders of the upper and lower airway is nasal polyposis.

6. The method of claim 1, wherein the subject has nasal polyps.

7. The method of claim 6, wherein tissue from the subject comprising the nasal polyps comprises eosinophilic infiltration.

8. The method of claim 1, wherein the subject has received a first line treatment comprising intranasal corticosteroids for the one or more disorders of the upper and lower airway.

9. The method of claim 1, wherein the inhibitor of ALOX15 comprises a small molecule.

10. The method of claim 1, wherein the inhibitor of ALOX15 comprises RNAi.

11. The method of claim 10, wherein the RNAi inhibits translation or degrades ALOX15 mRNA.

12. The method of claim 10, wherein the RNAi comprises siRNA, miRNA, or antisense oligonucleotide (ASO).

13. The method of claim 12, wherein the ASO is single-stranded or double-stranded.

14. The method of claim 1, wherein the inhibitor of ALOX15 is an aptamer.

15. The method of claim 14, wherein the aptamer is an oligonucleotide or a peptide molecule.

16. The method of claim 1, wherein the subject comprises an ALOX15 variant.

17. The method of claim 16, wherein the ALOX15 variant is rs2255888.

18. The method of claim 1, wherein the inhibitor of ALOX15 causes a reduction in the production of a metabolite of ALOX15 in the subject.

19. The method of claim 18, wherein the metabolite of ALOX15 is 15-hydroxyeicosatetraenoic acid (15-HETE).

20. The method of claim 1, wherein the inhibitor of ALOX15 causes a reduction in the subject of blood eosinophil counts.

21. A composition comprising an inhibitor of ALOX15 that is efficacious in treating nasal polyposis, chronic sinusitis, allergic rhinitis, asthma, or NSAID-exacerbated respiratory disease.

22. The composition of claim 21, wherein the inhibitor of ALOX15 is an RNAi.

23. The composition of claim 21, wherein the RNAi is siRNA.

24. The composition of claim 21, wherein the RNAi is miRNA.

25. The composition of claim 21, wherein the RNAi is an antisense oligonucleotide (ASO).

26. The composition of claim 25, wherein the ASO is double-stranded or single-stranded.

27. The composition of claim 21, wherein the inhibitor of ALOX15 is a small molecule.

28. The composition of claim 21, wherein the inhibitor of ALOX15 is an aptamer.

29. The composition of claim 28, wherein the aptamer is an oligonucleotide aptamer.

30. The composition of claim 28, wherein the aptamer is a peptide aptamer.

31. A method of treating one or more disorders of the upper and lower airway in a subject in need thereof comprising editing an ALOX15 gene in the subject wherein the one or more disorders of the upper and lower airway comprises nasal polyposis, chronic sinusitis, allergic rhinitis, asthma, or NSAID-exacerbated respiratory disease.

32. The method of claim 31, wherein the editing of the ALOX15 gene comprises administering CRISPR/cas9 to the subject.

33. The method of claim 32, wherein the CRISPR/cas9 targets the ALOX15 gene.

34. The method of claim 32, wherein the CRISPR/cas9 edits the ALOX15 gene to a loss of function mutation.

35. The method of claim 34, wherein the loss of function mutation comprises a threonine to methionine mutation.

36. The method of claim 35, wherein the threonine to methionine mutation occurs at amino acid position 560 according to the human protein sequence numbering.

37. The method of claim 32, wherein the CRISPR/cas9 is delivered systemically to the subject.

38. The method of claim 32, wherein the CRISPR/cas9 is delivered locally to the subject.

39. The method of claim 38, wherein the CRISPR/cas9 is delivered locally to the nasal epithelium of the subject.

40. The method of claim 32, wherein the editing of the ALOX15 gene is efficacious in treating the one or more disorders of the upper and lower airway.

41. The method of claim 31, wherein the one or more disorders of the upper and lower airway is nasal polyposis.

42. The method of claim 31, wherein the subject has nasal polyps.

43. The method of claim 42, wherein tissue from the subject comprising the nasal polyps comprises eosinophilic infiltration.

44. The method of claim 31, wherein the subject has received a first line treatment comprising intranasal corticosteroids for the one or more disorders of the upper and lower airway.

45. The method of claim 31, wherein the editing of the ALOX15 gene causes a reduction in the production of a metabolite of ALOX15 in the subject.

46. The method of claim 45, wherein the metabolite of ALOX15 is 15-hydroxyeicosatetraenoic acid (15-HETE).

47. The method of claim 31, wherein the editing of the ALOX15 gene causes a reduction in the subject of blood eosinophil counts.

48. A composition comprising CRISPR/cas9 that targets ALOX15 that is efficacious in treating nasal polyposis, chronic sinusitis, allergic rhinitis, asthma, or NSAID-exacerbated respiratory disease.

49. The composition of claim 48, wherein the CRISPR/cas9 edits the ALOX15 gene to a loss of function mutation.

50. The composition of claim 49, wherein the loss of function mutation comprises a threonine to methionine mutation.

51. The composition of claim 50, wherein the threonine to methionine mutation occurs at amino acid position 560 according to the human protein sequence numbering.
